SEQUENCE LISTING (19 sequences)

SEQ ID NO: 1 - protein, 21 aa, Homo sapiens
YRQSMNNFQGLRSFGCRFGTC

SEQ ID NO: 2 - protein, 8 aa, Synthetic construct
GYTFSRYW

SEQ ID NO: 3 - protein, 8 aa, Synthetic construct
ILPGSGST

SEQ ID NO: 4 - protein, 11 aa, Synthetic construct
TEGYEYDGFDY

SEQ ID NO: 5 - protein, 11 aa, Synthetic construct
QSIVYSNGNTY

SEQ ID NO: 6 - skipped

SEQ ID NO: 7 - protein, 9 aa, Synthetic construct
FQGSHIPYT

SEQ ID NO: 8 - protein, 448 aa, Synthetic construct
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, Synthetic construct
DVVLTQSPLSLPVTLGQPASISCRSSQSIVYSNGNTYLEWYLQRPGQSPRLLIYRVSNRFSGVPDRFSGSGSGTDFTLKISRVEAEDVGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 20 aa, Homo sapiens
ARLDVASEFRKKWNKWALSR

SEQ ID NO: 11 - protein, 48 aa, Homo sapiens
ELRMSSSYPTGLADVKAGPAQTLIRPQDMKGASRSPEDSSPDAARIRV

SEQ ID NO: 12 - protein, 38 aa, Homo sapiens
RRRRRSLPEAGPGRTLVSSKPQAHGAPAPPSGSAPHFL

SEQ ID NO: 13 - protein, 52 aa, Homo sapiens
  MOD_RES 52: note = C-terminal amidation
YRQSMNNFQGLRSFGCRFGTCTVQKLAHQIYQFTDKDKDNVAPRSKISPQGY

SEQ ID NO: 14 - protein, 53 aa, Homo sapiens
YRQSMNNFQGLRSFGCRFGTCTVQKLAHQIYQFTDKDKDNVAPRSKISPQGYG

SEQ ID NO: 15 - protein, 14 aa, Homo sapiens
YRQSMNNFQGLRSF

SEQ ID NO: 16 - protein, 10 aa, Homo sapiens
YRQSMNNFQG

SEQ ID NO: 17 - protein, 6 aa, Homo sapiens
YRQSMN

SEQ ID NO: 18 - protein, 42 aa, Homo sapiens
YRQSMNNFQGLRSFGCRFGTCTVQKLAHQIYQFTDKDKDNVA

SEQ ID NO: 19 - protein, 10 aa, Homo sapiens
  MOD_RES 10: note = C-terminal amidation
PRSKISPQGY

(19) United States
(12) Patent Application Publication
(10) Pub. No.: US 2025/0304671 A1
(43) Pub. Date: Oct. 2, 2025

(54) STABLE AQUEOUS FORMULATION OF AN ANTI-ADRENOMEDULLIN (ADM) ANTIBODY OR ANTI-ADM ANTIBODY FRAGMENT

(71) Applicant: ADRENOMED AG, Hennigsdorf (DE)

(72) Inventor: Jarrid GOLDSTEIN, München (DE)

(73) Assignee: ADRENOMED AG, Hennigsdorf (DE)

(21) Appl. No.: 18/844,746

(22) PCT Filed: Mar. 15, 2023
(86) PCT No.: PCT/EP2023/056663

§ 371 Date: Sep. 6, 2024

(30) Foreign Application Priority Data
Mar. 15, 2022 (EP) 22162271.5

(51) Int. Cl. C07K 16/22; A61K 9/08; A61K 47/18; A61K 47/22; A61K 47/26

(52) U.S. Cl. CPC C07K 16/22; A61K 9/08; A61K 47/183; A61K 47/22; A61K 47/26; C07K 2317/21; C07K 2317/24

(57) ABSTRACT

Subject matter of the present invention are aqueous pharmaceutical formulations of antibodies. Specifically, the present invention relates to a stable aqueous antibody formulation comprising arginine, trehalose, a surfactant and histidine and its pharmaceutical preparation and use. This invention is exemplified by an aqueous formulation of an anti-ADM antibody or anti-ADM antibody fragment.

The present invention relates to the field of pharmaceutical formulations of antibodies. Specifically, the subject matter of the present invention relates to a stable liquid antibody formulation comprising Arginine, Trehalose, a surfactant and Histidine and its pharmaceutical preparation and use.

### BACKGROUND

Antibody preparations used for therapeutic or prophylactic use require a stabilizer to prevent loss of protein activity or structural integrity due to the effects of denaturation, oxidation, or aggregation over a period of time during storage and transportation before use. These problems are exacerbated by the presence of high levels of antibodies that are often desirable for treatment of diseases. The main objectives in the development of antibody formulations are to maintain the antibody, its solubility, stability, and antigen-binding potency. It is particularly important to avoid agglomeration of particles in solution, which requires aseptic filtration prior to use for intravenous or subcutaneous administration and will limit the route of administration. The formulation of antibody preparations requires careful selection of these factors among others to avoid protein denaturation and loss of antigen-binding activity. Therefore, there is a need for a stable aqueous antibody formulation that supports stable high concentrations of the bioactive antibody in solution and is suitable for parenteral administration, including intravenous, intramuscular, intraperitoneal, intradermal, or subcutaneous injection.

Furthermore, diverse diseases or illnesses may have common, partially non-specific symptoms that can range from unpleasant to unbearable for the individual suffering from therefrom. Quite often individuals experiencing more than one symptom need to take several drugs to experience alleviation of these symptoms. There is an ongoing need for new forms of therapy or prevention of symptoms associated with many different underlying diseases or illnesses. In particular, it would be helpful to provide medicaments or drugs that can be used in the therapy or prevention of more than one symptom associated with an underlying disease or illness.

The peptide adrenomedullin (ADM) was described for the first time in 1993 (Kitamura K. et al. 1993. Biochemical and Biophysical Research Communications Vol. 192 (2): 553-560) as a novel hypotensive peptide comprising 52 amino acids, which had been isolated from a human pheochromocytome.

In the same year, cDNA coding for a precursor peptide comprising 185 amino acids and the complete amino acid sequence of this precursor peptide were also described.

The precursor peptide, which comprises, inter alia, a signal sequence of 21 amino acids at the N-terminus, is referred to as “preproadrenomedullin” (pre-proADM). In the present description, all amino acid positions specified usually relate to the pre-proADM which comprises the 185 amino acids. The peptide adrenomedullin (ADM) is a peptide which comprises 52 amino acids (SEQ ID NO: 13) and which comprises the amino acids 95 to 146 of pre-proADM, from which it is formed by proteolytic cleavage. To date, only a few fragments of the peptide fragments formed in the cleavage of the pre-proADM have been more exactly characterized, in particular the physiologically active peptides adrenomedullin (ADM) and “PAMP”, a peptide comprising 20 amino acids (22-41) which follows the 21 amino acids of the signal peptide in pre-proADM. The discovery and characterization of ADM in 1993 triggered intensive research activity, the results of which have been summarized in various review articles, in the context of the present description, reference being made in particular to the articles to be found in an issue of “Peptides” devoted to ADM in particular (Editorial, Takahashi K. 2001. Peptides 22:1691) and (Eto T. 2001. Peptides 22: 1693-1711). A further review is (Hinson et al. 2000, Endocrine Reviews 21(2):138-167). In the scientific investigations to date, it has been found, inter alia, that ADM may be regarded as a poly-functional regulatory peptide. It is released into the circulation in an inactive form extended by glycine (Kitamura K. et al. 1998. Biochem. Biophys. Res. Commun. 244(2):551-555). There is also a binding protein (Pio R. et al. 2001. The Journal of Biological Chemistry 276(15):12292-12300) which is specific for ADM and probably likewise modulates the effect of ADM. Those physiological effects of ADM as well as of PAMP which are of primary importance in the investigations to date were the effects influencing blood pressure.

ADM is an effective vasodilator, and it is possible to associate the hypotensive effect with the particular peptide segments in the C-terminal part of ADM. It has furthermore been found that the above-mentioned further physiologically active peptide PAMP formed from pre-proADM likewise exhibits a hypotensive effect, even if it appears to have an action mechanism differing from that of ADM.

It has furthermore been found that the concentrations of ADM, which can be measured in the circulation and other biological liquids are, in a number of pathological states, significantly above the concentrations to be found in healthy control persons. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM ((Eto, T., supra).

It is furthermore known that unusually high concentrations of ADM are to be observed in sepsis, and the highest concentrations in septic shock (cf. (Eto, T., “supra) and (Hirata et al. Journal of Clinical Endocrinology and Metabolism 1996. 81(4): 1449-1453; Ehlenz K. et al. 1997. Exp Clin Endocrinol Diabetes 105: 156-162); Tomoda Y. et al. 2001. Peptides 22: 1783-1794; Ueda S. et al. 1999. Am. J. Respir. Crit. Care Med. 160: 132-136; and Wang P. Peptides 2001. 22: 1835-1840).

WO-A1 2004/097423 describes the use of an antibody against adrenomedullin for diagnosis, prognosis, and treatment of cardiovascular disorders. Treatment of diseases by blocking the ADM receptor are also described in the art, (e.g., WO-A1 2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases.

It is reported for the early phase of sepsis that ADM improves heart function and the blood supply in liver, spleen, kidney and small intestine. ADM-neutralizing antibodies neutralize the before mentioned effects during the early phase of sepsis (Wang, P., “Adrenomedullin and cardiovascular responses in sepsis”, Peptides, Vol. 22, pp. 1835-1840 (2001).

For other diseases blocking of ADM may be beneficial to a certain extent. However, it might also be detrimental if ADM is totally neutralized, as a certain amount of ADM may be required for several physiological functions. In many reports it was emphasized, that the administration of ADM may be beneficial in certain diseases. In contrast thereto, in other reports ADM was reported as being life threatening when administered in certain conditions.

Administration of ADM in combination with ADM-binding-Protein-1 is described for treatment of sepsis and septic shock in the art. It is assumed that treatment of septic animals with ADM and ADM-binding-Protein-1 prevents transition to the late phase of sepsis. It has to be noted that in a living organism ADM binding protein (complement factor H) is present in the circulation of said organism in high concentrations (Pio et al.: Identification, characterization, and physiological actions of factor H as an Adrenomedullin binding Protein present in Human Plasma; Microscopy Res. and Technique, 55:23-27 (2002) and Martinez et al.; Mapping of the Adrenomedullin-Binding domains in Human Complement factor H; Hypertens Res Vol. 26, Suppl (2003), S56-59).

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Exp 1(1):22; Wagner et al. 2013. Intensive Care Med Exp 1(1):21).

Due to these positive results, a humanized version of an N-terminal anti-ADM antibody, named Adrecizumab, has been developed for further clinical development. Beneficial effects of Adrecizumab on vascular barrier function and survival were recently demonstrated in preclinical models of systemic inflammation and sepsis (Geven et al. 2018. Shock 50(6):648-654). In this study, pre-treatment with Adrecizumab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Adrecizumab improved 7-day survival in CLP-induced sepsis in mice from 10 to 50% for single and from 0 to 40% for repeated dose administration. Of particular interest is the proposed mechanism of action of Adrecizumab. Both animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2):132-140; and Voors et al (J. Eur J Heart Fail. 2019 February; 21(2):163-171)). In addition, binding of the antibody to ADM leads to a prolongation of ADM's half-life. Even though NT-ADM antibodies partially inhibit ADM-mediated signalling, a large increase of circulating ADM results in an overall “net” increase of ADM activity in the blood compartment, where it exerts beneficial effects on ECs (predominantly barrier stabilization), whereas ADMs detrimental effects on VSMCs (vasodilation) in the interstitium are reduced.

WO2013/072510 describes a non-neutralizing anti-ADM antibody for use in therapy of a severe chronical or acute disease or acute condition of a patient for the reduction of the mortality risk for said patient.

WO2013/072511 describes a non-neutralizing anti-ADM antibody for use in therapy of a chronical or acute disease or acute condition of a patient for prevention or reduction of organ dysfunction or organ failure.

WO2013/072512 describes a non-neutralizing anti-ADM antibody that is an ADM stabilizing antibody that enhances the half-life (t_{1/2} half retention time) of adrenomedullin in serum, blood, plasma. This ADM stabilizing antibody blocks the bioactivity of ADM to less than 80%.

WO2013/072513 shows that in patients having a chronic or acute disease or acute condition in need for stabilizing the circulation, anti-Adrenomedullin (ADM) antibody or an anti-adrenomedullin antibody fragment or an anti-ADM non-lg scaffold stabilizes the blood circulation of patients and reduces the vasopressor requirement, e.g., catecholamine of said patient.

WO2013/072514 shows anti-Adrenomedullin (ADM) antibody or an anti-adrenomedullin antibody fragment or an anti-ADM non-lg scaffold can be efficiently used to regulate the fluid balance in a patient having a chronic or acute disease or acute condition, especially patients at the ICU (Intensive Care Unit) who suffers from fluid imbalance.

WO2017/182561 describes methods for determining the total amount or active DPP3 in a sample of a patient for the diagnosis of a disease related to necrotic processes. It further describes a method of treatment of necrosis-related diseases by antibodies directed to DPP3.

The inventors have now found that treatment of anti-adrenomedullin (ADM) antibody or an anti-adrenomedullin antibody fragment can be particularly effective in therapy of a patient suffering from diseases associated with impaired vascular integrity.

Consequently, there is a need to provide a stable aqueous formulation for an anti-adrenomedullin (ADM) antibody.

### DETAILED DESCRIPTION OF THE INVENTION

Although the present invention will be described with respect to particular embodiments, this description is not to be construed in a limiting sense.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting” of is considered to be a preferred embodiment of the term “comprising” of. If hereinafter a group is defined to comprise at least a certain number of embodiments, this is meant to also encompass a group, which preferably consists of these embodiments only.

It is also to be understood that the terminology used herein is for the purpose of describing particular embodiments only and is not intended to limit the scope of the present invention that will be limited only by the appended claims.

Below, embodiments of the invention are provided. It is noted that generally embodiments can be combined with any other embodiment of the same category (product, process, use, method).

Subject matter of the present invention is a pharmaceutical aqueous formulation comprising an human or humanized anti-adrenomedullin (ADM) antibody or an anti-adrenomedullin antibody fragment wherein said antibody or fragment binds to the N-terminal part (aa 1-21) of ADM: YRQSMNNFQGLRSFGCRFGTC (SEQ ID No. 1), wherein said antibody or antibody fragment is present in a concentration of 1 mg/ml to 100 mg/ml, 2 mg/ml to 50 mg/ml, more preferably 10 mg/ml to 30 mg/ml, more preferably 15 mg/ml to 25 mg/ml and most preferably 20 mg/ml and wherein said pharmaceutical aqueous formulation is further comprising:
Arginine in a range of 1 g/L to 100 g/L, and
Trehalose in a range of 1 g/L to 100 g/L, and
A surfactant selected from polysorbates and poloxamers in a range of 0.01 g/L to 5 g/L, and
Histidine in a range of 0.1 g/L to 6.4 g/L, and
wherein the pH of the formulation is in the range of 4.0 to 8.0.

A preferred embodiment of the present invention is a pharmaceutical aqueous formulation comprising an human or humanized anti-adrenomedullin (ADM) antibody or an anti-adrenomedullin antibody fragment wherein said antibody or fragment binds to the N-terminal part (aa 1-21) of ADM: YRQSMNNFQGLRSFGCRFGTC (SEQ ID No. 1), wherein said antibody or antibody fragment is present in a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml and wherein said pharmaceutical aqueous formulation is further comprising:
Arginine or a salt thereof in a range of 1 g/L to 100 g/L based on content of arginine zwitter ion, and
Trehalose in a range of 1 g/L to 100 g/L, and
A surfactant selected from polysorbates such as ethoxylated sorbitan esterified with fatty acids comprising Polysorbate 20, Polysorbate 40, Polysorbate 60, and Polysorbate 80 and poloxamers such as copolymers based on ethylene oxide and propylene oxide in a range of 0.01 g/L to 5 g/L, and
Histidine in a range of 0.1 g/L to 6.4 g/L, and
wherein the pH of the formulation is in the range of 4.0 to 8.0.

In a particular embodiment of the present invention, the ADM antibody or an ADM antibody fragment may be administered in a dose of at least 0.5 mg/Kg body weight, particularly at least 1.0 mg/kg body weight, more particularly, from 1.0 to 20.0 mg/kg body weight, e.g., from 1.0 to 10 mg/kg, from 2.0 to 10 mg/kg body weight, from 2.0 to 8.0 mg/kg body weight or from 3.0 to 5.0 mg/kg body weight.

In a particular embodiment of the present invention, the ADM antibody or an ADM antibody fragment is present in a concentration of at least of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml.

In a further embodiment of the present invention, the ADM antibody or an ADM antibody fragment is present in a total amount of at least 5 mg, particularly at least 10 mg, more particularly from 10 mg to 1000 mg, more particularly from 50 mg to 700 mg, more particularly from 100 mg to 500 mg and most particularly from 200 to 480 mg referring to a 10 ml vial. This total amount of the ADM antibody or an ADM antibody fragment can e.g., be used to prepare a ready-to-application solution by diluting said total amount of the ADM antibody or an ADM antibody fragment in a suitable buffer comprising phosphate buffered saline solution, water and emulsions such as oil/water emulsion to a desired concentration. In an embodiment of the present invention, the ADM antibody or an ADM antibody fragment is present in a total amount of at least 5 mg, particularly at least 10 mg, more particularly from 10 mg to 1000 mg, more particularly from 50 mg to 700 mg, more particularly from 100 mg to 500 mg and most particularly from 200 to 480 mg referring to a 10 ml vial and may be diluted in a suitable buffer comprising phosphate buffered saline solution, water and emulsions such as oil/water emulsion to 10 ml to 200 ml, preferably 20 ml to 180 ml, more preferably 30 ml to 150 ml and most preferred to 50 ml to 100 ml.

In another embodiment of the present invention, the ADM antibody or an ADM antibody fragment is present in a ready-to-application solution with a concentration of at least 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml and diluted in a suitable buffer comprising phosphate buffered saline solution, water and emulsions such as oil/water emulsion to 10 ml to 200 ml, preferably 20 ml to 180 ml, more preferably 30 ml to 150 ml and most preferred to 50 ml to 100 ml.

The person skilled in the art would then know from instructions for use how to prepare a ready-for-application solution from the provided pharmaceutical aqueous formulation, which can then be applied to the patient by known routes of administration.

In other embodiments, the pharmaceutical aqueous formulation is further diluted in an infusion solution and applied via infusion to the patient. In another embodiment, the physician as person skilled in the art would prepare a ready-for-application solution from the pharmaceutical aqueous formulation according to the patient's needs and then directly apply the ready-for-application solution to the patient. In an embodiment of the present invention, the ADM antibody or an ADM antibody fragment may be administered in a dose of at least 0.5 mg/kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight or from 3.0 to 5.0 mg/kg body weight.

Subject matter of the present invention is a pharmaceutical aqueous formulation comprising an human or humanized anti-adrenomedullin (ADM) antibody or an anti-adrenomedullin antibody fragment wherein said antibody or fragment is a human monoclonal antibody or fragment that binds to the N-terminal region (aa 1-21) of ADM (SEQ ID No. 1) or an antibody fragment thereof wherein the heavy chain comprises the sequences:

CDR1:
SEQ ID NO: 2
GYTFSRYW
CDR2:
SEQ ID NO: 3
ILPGSGST
CDR3:
SEQ ID NO: 4
TEGYEYDGFDY

and wherein the light chain comprises the sequences:

CDR1:
SEQ ID NO: 5
QSIVYSNGNTY
CDR2:
SEQ ID NO: 6
RVS
CDR3:
SEQ ID NO: 7
FQGSHIPYT.

Subject matter of the present invention is a pharmaceutical aqueous formulation comprising an human or humanized anti-adrenomedullin (ADM) antibody or an anti-adrenomedullin antibody fragment comprising an Anti-Adrenomedullin antibody directed to the N-terminal end of Adrenomedullin comprising the following sequence as a heavy chain:

SEQ ID NO: 8
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

or a sequence that is >95% identical to it,
and comprises the following sequence as a light chain:

SEQ ID NO: 9
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >95% identical to it.

In a further embodiment of the present invention, a pharmaceutical aqueous formulation comprising an human or humanized anti-adrenomedullin (ADM) antibody or an anti-adrenomedullin antibody fragment comprising an Anti-Adrenomedullin antibody directed to the N-terminal end of Adrenomedullin comprising the following sequence as a heavy chain:

SEQ ID NO: 8
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

or a sequence that is >95% identical to it, preferably >98%, preferably >99%
and comprises the following sequence as a light chain:

SEQ ID NO: 9
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >95% identical to it, preferably >98%, preferably >99%.

To assess the identity between two amino acid sequences, a pairwise alignment is performed. The identity defines the percentage of amino acids with a direct match in the alignment.

Surfactants, as disclosed in the present invention, can be used to alter the surface tension of a liquid antibody formulation. In a special embodiment, the surfactant reduces the surface tension of a liquid antibody formulation. In another embodiment, the surfactant can contribute to an improvement in stability of any of the antibody in the formulation. The surfactant can also reduce aggregation of the formulated antibody (e.g., during shipping and storage) and/or minimize the formation of particulates in the formulation and/or reduces adsorption (e.g., adsorption to a container). As an example, the surfactant can also improve stability of the antibody during and after a freeze/thaw cycle. The surfactant can be, for example without limitation, a polysorbate, poloxamer, triton, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof.

Subject matter of the present invention is a pharmaceutical aqueous formulation comprising an human or humanized anti-adrenomedullin (ADM) antibody or an anti-adrenomedullin antibody fragment wherein said surfactant is Poloxamer. In another embodiment poloxamer is selected from the group comprising copolymers based on ethylene oxide and propylene oxide comprising but not limited to L64, P65, P84, P85, F88, P103, P104, P105, F108, P123 or F127.

In some embodiments, the surfactant selected from polysorbate and poloxamers wherein the polysorbates are derived from ethoxylated sorbitan esterified with fatty acids and selected from the group comprising Polyoxyethylen-(20)-sorbitanmonolaurate, Polyoxyethylen-(4)-sorbitanmonolaurate, Polyoxyethylen-(20)-sorbitanmonopalmitate, Polyoxyethylen-(20)-sorbitanmonostearate, Polyoxyethylen-(4)-sorbitanmonostearate, Polyoxyethylen-(20)-sorbitantristearate, Polyoxyethylen-(20)-sorbitanmonooleate, Polyoxyethylen-(5)-sorbitanmonooleate, Polyoxyethylen-(20)-sorbitantrioleate or Polyoxyethylen-(20)-sorbitanmonoisostearate, polyoxyethylene derivatives, Tween, PEG3350 and mixtures thereof.

Subject matter of the present invention is a pharmaceutical aqueous formulation comprising an human or humanized anti-adrenomedullin (ADM) antibody or an anti-adrenomedullin antibody fragment wherein said formulation is essentially free of NaCl and/or Glycine.

In some embodiments the aqueous formulation comprises for example without limitation, acetate, succinate, gluconate, citrate, histidine, acetic acid, phosphate, phosphoric acid, ascorbate, tartaric acid, maleic acid, glycine, lactate, lactic acid, ascorbic acid, imidazole, bicarbonate and carbonic acid, succinic acid, sodium benzoate, benzoic acid, gluconate, edetate, acetate, malate, imidazole, tris, phosphate, and mixtures thereof. Preferably the aqueous formulation comprises histidine, wherein the histidine can comprise either L-histidine or D-histidine, a solvated form of histidine, a hydrated form (e.g., monohydrate including L-histidine hydrochloride monohydrate) of histidine, a salt of histidine (e.g., histidine hydrochloride) or an anhydrous form of histidine or a mixture thereof.

Subject matter of the present invention is a pharmaceutical aqueous formulation comprising an human or humanized anti-adrenomedullin (ADM) antibody or an anti-adrenomedullin antibody fragment wherein Arginine is present in a range of 1 g/L to 100 g/L, preferably 13.1 g/L to 52.2 g/L, more preferably 26.1 g/L. Preferably the aqueous formulation comprises histidine, wherein the histidine can comprise either L-histidine or D-histidine, a solvated form of histidine, a hydrated form (e.g., monohydrate including L-histidine hydrochloride monohydrate) of histidine, a salt of histidine (e.g., histidine hydrochloride) or an anhydrous form of histidine or a mixture thereof.

In one aspect of the present invention, the aqueous formulation comprises an isotonicity modifying agent which protects the antibody or protein in the formulation against freeze-thaw induced aggregation as well as aggregation on storage. Said isotonicity agent can be a polyol with a molecular weight that, for example without limitation, is less than about 600 kD (e.g., in the range from about 120 to about 400 kD), and comprises multiple hydroxyl groups including sugars (e.g., reducing and nonreducing sugars or mixtures thereof, saccharide, or a carbohydrate), sugar alcohols, sugar acids, or a salt or mixtures thereof. Examples of non-reducing sugar include, but are not limited to, sucrose, trehalose, and mixtures thereof. In some embodiments, the polyol is mannitol, trehalose, sorbitol, erythritol, isomalt, lactitol, maltitol, xylitol, glycerol, lactitol, propylene glycol, polyethylene glycol, inositol, or mixtures thereof. In other embodiments, the polyol can be, for example without limitation, a monosaccharide, disaccharide or polysaccharide, or mixtures of any of the foregoing. The saccharide or carbohydrate can be, for example without limitation, fructose, glucose, mannose, sucrose, sorbose, xylose, lactose, maltose, sucrose, dextran, pullulan, dextrin, cyclodextrins, soluble starch, hydroxyethyl starch, water-soluble glucans, or mixtures thereof. In a preferred embodiment of the present invention the polyol is trehalose.

Subject matter of the present invention is pharmaceutical aqueous formulation comprising an human or humanized anti-adrenomedullin (ADM) antibody or an anti-adrenomedullin antibody fragment wherein Trehalose is present in a range of 1 g/L to 100 g/L, preferably 28.4 g/L to 85.1 g/L, more preferably 56.7 g/L.

Subject matter of the present invention is a pharmaceutical aqueous formulation comprising an human or humanized anti-adrenomedullin (ADM) antibody or an anti-adrenomedullin antibody fragment wherein Poloxamer is present in a range of 0.01 g/L to 5 g/L, preferably 0.1 g/L to 1.0 g/L, more preferably 0.5 g/L.

In some embodiments, the aqueous formulation comprises a chelating agent that can be selected from the group consisting of aminopolycarboxylic acids, hydroxyaminocarboxylic acids, N-substituted glycines, 2-(2-amino-2-oxoethyl) aminoethane sulfonic acid (BES), deferoxamine (DEF), citric acid, niacinamide, and desoxycholates and mixtures thereof. In some embodiments, the chelating agent is selected from the group consisting of ethylenediaminetetraacetic acid (EDTA), diethylenetriamine pentaacetic acid 5 (DTPA), nitrilotriacetic acid (NTA), N-2-acetamido-2-iminodiacetic acid (ADA), bis(aminoethyl)glycol ether, N, N,N′, N′-tetra acetic acid (EGTA), trans-diamino cyclohexane tetra acetic acid (DCTA), glutamic acid, and aspartic acid, N-hydroxyethyl iminodiacetic acid (HIMDA), N,N-bis-hydroxyethyl glycine (bicine) and N-(trishydroxymethylmethyl) 10 glycine (tricine), glycylglycine, sodium desoxycholate, ethylenediamine, propylenediamine, diethylenetriamine, triethylenetetramine (trien), disodium edetate dihydrate (or disodium EDTA dihydrate or EDTA disodium salt), calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In some embodiments, the chelating agent is selected from the group consisting of salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents used in the invention can be present, where possible, as the free acid or free base form or salt form of the compound, also as an anhydrous, solvated or hydrated form of the compound or corresponding salt. In a most preferred embodiment of the present invention the chelating agent is histidine.

Subject matter of the present invention is a pharmaceutical aqueous formulation comprising a human or humanized anti-adrenomedullin (ADM) antibody or an anti-adrenomedullin antibody fragment wherein Histidine is present in a range of 0.1 g/L to 6.4 g/L, preferably 0.8 g/L to 3.2 g/L, more preferably 1.6 g/L.

Subject matter of the present invention is a pharmaceutical aqueous formulation comprising a human or humanized anti-adrenomedullin (ADM) antibody or an anti-adrenomedullin antibody fragment wherein the pH of the formulation is in the range of 4.0 to 8.0 preferably 5.0 to 7.0, more preferably 6.0.

According to the present invention, the aqueous formulation is provided with a pH close to physiological pH for reduced risk of pain or anaphylactoid side effects on injection and also provides enhanced antibody stability and resistance to aggregation, oxidation, and fragmentation.

In some embodiments the formulation can comprise a preservative. Preferably the preservative agent is selected from the group comprising phenol, m-cresol, benzyl alcohol, benzalkonium chloride, benzalkonium chloride, phenoxyethanol and methyl paraben.

Subject matter of the present invention is a pharmaceutical aqueous formulation comprising an human or humanized anti-adrenomedullin (ADM) antibody or an anti-adrenomedullin antibody fragment wherein said formulation comprises an Anti-Adrenomedullin antibody directed to the N-terminal end of Adrenomedullin comprising the following sequence: SEQ ID No. 1, Arginine in a concentration of 26.1 g/L, Trehalose in a concentration of 56.7 g/L, Poloxamer in a concentration of 0.5 g/L, Histidine in a concentration of 1.6 g/L and wherein said formulation exhibits a pH of 6,0.

In another embodiment of the present invention, pharmaceutical aqueous formulation comprising an human or humanized anti-adrenomedullin (ADM) antibody or an anti-adrenomedullin antibody fragment wherein said formulation comprises an Anti-Adrenomedullin antibody directed to the N-terminal end of Adrenomedullin comprising the following sequence: SEQ ID No. 1, Arginine in a concentration of 1 g/l to 100 g/L, Trehalose in a concentration of 1 g/l to 100 g/L, Poloxamer in a concentration of 0.01 g/L to 5 g/L, Histidine in a concentration of 0.1 g/L to 6.4 g/L and wherein said formulation exhibits a pH in a range of 4.0 to 8.0.

Another aspect of the present invention relates to a pharmaceutical aqueous formulation comprising an human or humanized anti-adrenomedullin (ADM) antibody or an anti-adrenomedullin antibody fragment wherein said formulation comprises an Anti-Adrenomedullin antibody directed to the N-terminal end of Adrenomedullin comprising the following sequence: SEQ ID No. 1, Arginine in a concentration of 13.1 g/l to 52.2 g/L, Trehalose in a concentration of 28.4 g/L to 85.1 g/L, Poloxamer in a concentration of 0.1 g/L to 1 g/L, Histidine in a concentration of 0.8 g/L to 3.2 g/L and wherein said formulation exhibits a pH of 5.0 to 7.0.

Subject matter of the present invention is a pharmaceutical aqueous formulation comprising an human or humanized anti-adrenomedullin (ADM) antibody or an anti-adrenomedullin antibody fragment wherein said formulation is stable after storage for at least 4 weeks at 2 to 8° C. and less than 5 wt/wt-%, preferably 4 wt/wt-%, more preferably 3 wt/wt-%, more preferably 2 wt/wt-%, more preferably 1 wt/wt-%, more preferably 0.05%-mol or most preferably 0.01 wt/wt-% of the total antibody is aggregated as measured for example by size exclusion high performance liquid chromatography (SEC-HPLC) and wherein said antibody is present at a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml. Other methods used in the characterization of molecular weight distribution of biological macromolecules are for example Micellar liquid chromatography or ion-exchange chromatography.

A preferred embodiment of the present invention is a pharmaceutical aqueous formulation comprising an human or humanized anti-adrenomedullin (ADM) antibody or an anti-adrenomedullin antibody fragment wherein said formulation is stable after storage for at least 4 weeks at 2 to 8° C. and wherein less than 5 wt/wt-%, preferably 4 wt/wt-%, more preferably 3 wt/wt-%, more preferably 2 wt/wt-%, more preferably 1 wt/wt-%, more preferably 0.05%-mol or most preferably 0.01 wt/wt-% of the total antibody is aggregated as measured for example by size exclusion high performance liquid chromatography (SEC-HPLC) and wherein said antibody may be administered in a dose of 1 mg/kg body weight to 10 mg/kg body weight, more preferably 2 mg/kg body weight to 8 mg/kg body weight and most preferably 3 mg/kg body weight to 5 mg/kg body weight. Other methods used in the characterization of molecular weight distribution of biological macromolecules are for example Micellar liquid chromatography or ion-exchange chromatography.

Subject matter of the present invention is a pharmaceutical aqueous formulation comprising an human or humanized anti-adrenomedullin (ADM) antibody or an anti-adrenomedullin antibody fragment wherein said formulation is stable after storage for at least 4 weeks at 2 to 8° C. and wherein less than 5 wt/wt-%, preferably 4 wt/wt-%, more preferably 3 wt/wt-%, more preferably 2 wt/wt-%, more preferably 1 wt/wt-%, more preferably 0.05%-mol or most preferably 0.01 wt/wt-% of the total antibody is aggregated as measured for example by size exclusion high performance liquid chromatography (SEC-HPLC) and wherein said antibody is present at a total amount of 10 mg to 1000 mg, more preferably 50 mg to 700 mg and most preferably 100 mg to 500 mg referring to a 10 ml vial. Other methods used in the characterization of molecular weight distribution of biological macromolecules are for example Micellar liquid chromatography or ion-exchange chromatography.

In an embodiment of the invention SEC-HPLC is capable to separate protein species having apparent molecular weights of 150 kDa from others having molecular weights of 300 kDa or higher.

Subject matter of the present invention is a pharmaceutical aqueous formulation comprising an human or humanized anti-adrenomedullin (ADM) antibody or an anti-adrenomedullin antibody fragment wherein the activity of said antibody or antibody fragment in said formulation is stable after stress and wherein stress is induced by storing said formulation for at least 4 weeks at 45° C., preferably 42° C., more preferably, 38° C., more preferably 36° C. and most preferably 40° C. and 85% rH, preferably 80 rH, preferably 70% rH, and most preferably 75% rH and/or by storing said formulation for at least 3 months of storage at 29° C., preferably 27° C., more preferably, 22° C., more preferably 20° C. and most preferably 25° C. and/or 70% rH, preferably 65% rH, preferably 55% rH, more preferably 50% rH and most preferably 60% rH and/or by storing said formulation for at least 6 months of storage at 9° C., preferably 7° C., more preferably, 2° C., more preferably 0° C. and most preferably 5° C. and/or by performing at least 5 Freeze/Thaw cycles with said formulation and/or by subjecting said formulation to mechanical stress comprising orbital shaking and overhead rotation and wherein the stability is determined by assessing the visual appearance comprising color, clarity and visible particles of said formulation after stress.

The abbreviation “rH” stands for “relative humidity and refers to a measure of how much water vapor is in a water-air mixture compared to the maximum amount possible. Furthermore, rH is a ratio of the humidity ratio of a particular water-air mixture compared to the saturation humidity ratio at a given temperature (dry-bulb). The term relative humidity “rH” is used according to the ICH Q1A guideline “Stability Testing of new Drug Substances and Products”. Because relative humidity is temperature dependent, precise air temperature control is required for close relative humidity control. The person skilled in the art would know that humidity is typically measured by a hygrometer such as a gravimetric hygrometer, chilled mirror hygrometer or electrolytic hygrometer.

“Stable” according to the present invention means that all critical quality attributes of the formulation remains within the specification limits of the quality specifications, in particular, the absence of any visible particulate after applied stress conditions such as storing said formulation for at least 2 weeks at 45° C., preferably 42° C., more preferably, 38° C., more preferably 36° C. and most preferably 40° C. and/or at 29° C., preferably 27° C., more preferably, 22° C., more preferably 20° C. and most preferably 25° C. and/or 70% rH, preferably 65% rH, preferably 55% rH, more preferably 50% rH and most preferably 60% rH and/or at 45° C., preferably 42° C., more preferably, 38° C., more preferably 36° C. and most preferably 40° C. and/or 85% rH, preferably 80 rH, preferably 70% rH, and most preferably 75% rH or by storing said formulation at 29° C., preferably 27° C., more preferably, 22° C., more preferably 20° C. and most preferably 25° C. and/or 70% rH, preferably 65% rH, preferably 55% rH, more preferably 50% rH and most preferably 60% rH or by storing said formulation at 9° C., preferably 7° C., more preferably, 2° C., more preferably 0° C. and most preferably 5° C. and/or by performing at least 5 Freeze/Thaw cycles with said formulation and/or by subjecting said formulation to mechanical stress comprising orbital shaking and overhead rotation.

In a further embodiment of the invention, pharmaceutical aqueous formulation comprising an human or humanized anti-adrenomedullin (ADM) antibody or an anti-adrenomedullin antibody fragment wherein the activity of said antibody or antibody fragment in said formulation is stable after stress and wherein stress is induced by storing said formulation for at least 2 weeks at 45° C., preferably 42° C., more preferably, 38° C., more preferably 36° C. and most preferably 40° C. and/or by performing at least 5 Freeze/Thaw cycles with said formulation and/or by subjecting said formulation to mechanical stress comprising orbital shaking and overhead rotation and wherein the stability is determined by assessing the visual appearance comprising color, clarity and visible particles of said formulation after stress.

In some embodiments of the present invention, pharmaceutical aqueous formulation comprising an human or humanized anti-adrenomedullin (ADM) antibody or an anti-adrenomedullin antibody fragment wherein the activity of said antibody or antibody fragment in said formulation is stable after stress and wherein stress is induced by storing said formulation for at least 2 months of storage at 29° C., preferably 27° C., more preferably, 22° C., more preferably 20° C. and most preferably 25° C. and/or 70% rH, preferably 65% rH, preferably 55% rH, more preferably 50% rH and most preferably 60% rH and/or by performing at least 5 Freeze/Thaw cycles with said formulation and/or by subjecting said formulation to mechanical stress comprising orbital shaking and overhead rotation and wherein the stability is determined by assessing the visual appearance comprising color, clarity and visible particles of said formulation after stress.

Another embodiment of the present invention relates to pharmaceutical aqueous formulation comprising an human or humanized anti-adrenomedullin (ADM) antibody or an anti-adrenomedullin antibody fragment wherein the activity of said antibody or antibody fragment in said formulation is stable after stress and wherein stress is induced by storing said formulation for at least 2 weeks at 45° C., preferably 42° C., more preferably, 38° C., more preferably 36° C. and most preferably 40° C. and/or 85% rH, preferably 80 rH, preferably 70% rH, and most preferably 75% rH or by storing said formulation at 29° C., preferably 27° C., more preferably, 22° C., more preferably 20° C. and most preferably 25° C. and/or 70% rH, preferably 65% rH, preferably 55% rH, more preferably 50% rH and most preferably 60% rH or by storing said formulation at 9° C., preferably 7° C., more preferably, 2° C., more preferably 0° C. and most preferably 5° C. and/or by performing at least 5 Freeze/Thaw cycles with said formulation and/or by subjecting said formulation to mechanical stress comprising orbital shaking and overhead rotation and wherein the stability is determined by assessing the visual appearance comprising color, clarity and visible particles of said formulation after stress. A person skilled in the art would also consider storing said formulation at the afore mentioned conditions for less than two weeks.

In another aspect of the present invention relate to at least 5 Freeze/Thaw cycles are performed wherein 1 Freeze/Thaw cycles comprises that said formulation is first frozen to −80° C. and subsequently thawed to 25° C. at 1° C. per minute. In some embodiments 1 to 50, preferably 1 to 30, more preferably 1 to 20, more preferably 1 to 10, more preferably 2 to 8 and most preferably 5 Freeze/Thaw cycles are performed.

In a further embodiment, pharmaceutical aqueous formulation comprising a human or humanized anti-adrenomedullin (ADM) antibody or an anti-adrenomedullin antibody fragment wherein the activity of said antibody or antibody fragment in said formulation is stable after stress and wherein stress is induced by subjecting said formulation to mechanical stress comprising orbital shaking and overhead rotation. In some embodiments, stress was applied by over-head rotation at 30 rpm for 24 h and/or orbital shaking at 400 rpm. In another aspect of the invention, stress was applied by over-head rotation at 30 rpm for 24 h, preferably for 36 h, more preferably for 48 h and most preferably for 72 h and/or orbital shaking at 200 rpm, preferably at 400 rpm, more preferably at 600 rpm, more preferably 800 rpm and most preferably at 1000 rpm.

Micron-sized protein aggregates and particles (subvisible particles, SVP) are important quality attributes of therapeutic protein formulations due to their risk of enhancing an immunogenic response. Quantification of subvisible particles larger than 10 μm and 25 μm is therefore required by the pharmacopoeias and is commonly performed using light obscuration (LO) techniques. In an embodiment of the present invention, the stability is determined by assessing the visual appearance comprising color, clarity and visible particles of said formulation after stress. In some embodiments of the invention, subvisible particles larger than 1 μm, more preferably 5 μm, more preferably 10 μm, more preferably 15 μm, more preferably 20 μm and most preferably 25 μm are quantified. In another embodiment, particles larger than 25 μm are quantified. In some embodiments, visual and color inspection is performed. Appearance testing of said formulation regarding color (according to Ph. Eur. method 2.2.2. Visible particles, Ph. Eur. method 2.9.20) clarity and degree of opalescence (Ph. Eur. method 2.2.) are usually analyzed according to Ph. Eur. method 2.9.20 and 2.2.1 respectively. In one aspect of the invention, visible particles, appearance, color and clarity are assessed by eye.

Subject matter of the present invention is a pharmaceutical aqueous formulation comprising a human or humanized anti-adrenomedullin (ADM) antibody or an anti-adrenomedullin antibody fragment wherein said formulation is stable after storage for at least 4 weeks at 2 to 8° C. and wherein stable means that said formulation remains free from visible particles.

Subject matter of the present invention is a pharmaceutical aqueous formulation comprising a human or humanized anti-adrenomedullin (ADM) antibody or an anti-adrenomedullin antibody fragment wherein said formulation is stable after storage for at least 4 weeks at 2 to 8° C. and wherein stable means that said formulation remains free from subvisible particles.

The person skilled in the art would know that micron-sized protein aggregates and particles (subvisible particles, SVP) are important quality attributes of therapeutic protein formulations due to their risk of enhancing an immunogenic response. Quantification of subvisible particles larger than 10 μm and 25 m is therefore required by the pharmacopoeias and is commonly performed using light obscuration (LO) techniques. Currently, the acceptance criteria for SVP are 6000 NMT≥10 μm/container and 600 NMT≥25 μm/container. SVPs<10 μm have to be monitored, acceptance criteria are not defined. However, quantification and characterization of particles with a size below 10 μm is of increasing interest and are in the meanwhile a regulatory expectation.

Subject matter of the present invention is a pharmaceutical aqueous formulation comprising a human or humanized anti-adrenomedullin (ADM) antibody or an anti-adrenomedullin antibody fragment wherein said antibody is present in a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml.

In some embodiments of the present invention, pharmaceutical aqueous formulation comprising an human or humanized anti-adrenomedullin (ADM) antibody or an anti-adrenomedullin antibody fragment wherein said antibody or said fragment may be administered in a dose of at least 0.5 mg/Kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight, or from 2.0 to 5.0 mg/kg body weight.

Subject matter of the present invention is a pharmaceutical lyophilized formulation obtainable from a pharmaceutical aqueous formulation. In some embodiments there is provided a formulation which is lyophilized and/or has been subjected to lyophilization. In another aspect of the invention, the pharmaceutical formulation is provided in a lyophilized form, which is termed a “cake” or “powder” and is physically stable and compatible with a pharmaceutically acceptable solvent mixture. In some embodiments there is provided a composition which is not lyophilized and has not been subjected to lyophilization.

In some embodiments, the aqueous formulation as disclosed by the present invention has a shelf life at 5° C. of at least or more than about 6 months, 12 months, 18 months, 24 months, 30 months, 36 months, 42 months, or 48 months (e.g. at 5° C., 25° C., or 40° C.). In a further embodiment, the formulation of the present invention has a shelf life of at least about 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months.

In some embodiments, the aqueous formulation as disclosed by the present invention has a shelf life at 25° C. of at least or more than about 6 months, 12 months, 18 months, 24 months, 30 months, 36 months, 42 months, or 48 months (e.g. at 5° C., 25° C., or 40° C.). In a further embodiment, the formulation of the present invention has a shelf life of at least about 6 months, 7 months, 8 months, 9 months, 10 months, 1 1 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months.

In some embodiments, the aqueous formulation as disclosed by the present invention has a shelf life at 40° C. of at least or more than about 6 months, 12 months, 18 months, 24 months, 30 months, 36 months, 42 months, or 48 months (e.g. at 5° C., 25° C., or 40° C.). In a further embodiment, the formulation of the present invention has a shelf life of at least about 6 months, 7 months, 8 months, 9 months, 10 months, 1 1 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months.

Subject matter of the present invention is a method of making a ready-for-application solution comprising the steps:
a. Providing a pharmaceutical aqueous formulation
b. Adjusting the pharmaceutical aqueous formulation from step a) in a physiological acceptable solution by optionally diluting the volume of the pharmaceutical formulation from step a) with a physiological acceptable solution and by optionally aliquoting the pharmaceutical formulation from step a) wherein the diluted or adjusted aqueous pharmaceutical formulation is suitable for the administration in a patient.

Subject matter of the present invention is a method of making a ready-for-application solution comprising the steps:
a. Providing a lyophilized formulation obtained from the aqueous formulation by freeze drying optionally without adding any other bulk reagent
b. Reconstitution of the lyophilizate from step a) in water and/or a physiological acceptable solution for injection
c. Adjusting the reconstituted formulation from step b) in a physiological acceptable solution by optionally diluting the volume of the reconstituted formulation from step b) with a physiological acceptable solution and by optionally aliquoting the reconstituted formulation from step b) wherein the diluted or adjusted aqueous pharmaceutical formulation is suitable for the administration in a patient.

A preferred embodiment of the present invention is a method of making a ready-to-use solution comprising the steps:
a. Providing a pharmaceutical aqueous formulation
b. Adjusting the pharmaceutical aqueous formulation from step a) in a physiological acceptable solution by optionally diluting the volume of the pharmaceutical formulation from step a) with a physiological acceptable solution and by optionally aliquoting the pharmaceutical formulation from step a) wherein the diluted or adjusted aqueous pharmaceutical formulation is suitable for the administration in a patient and wherein said solution is selected from the group comprising Ringer's lactate, glucose solution, electrolytic solutions, isotonic sodium chloride solution and wherein said infusion solution is present in a volume of 10 ml to 200 ml, preferably 20 ml to 180 ml, more preferably 30 ml to 150 ml and most preferred to 50 ml to 100 ml.

Another preferred embodiment of the present invention is a method of making a ready-to-use solution comprising the steps:
a. Providing a lyophilized formulation obtained from the aqueous formulation by freeze drying optionally without adding any other bulk reagent
b. Reconstitution of the lyophilizate from step a) in water and/or a physiological acceptable solution for injection
c. Adjusting the reconstituted formulation from step b) in a physiological acceptable solution by optionally diluting the volume of the reconstituted formulation from step b) with a physiological acceptable solution and by optionally aliquoting the reconstituted formulation from step b) wherein the diluted or adjusted aqueous pharmaceutical formulation is suitable for the administration in a patient
   and wherein said solution is selected from the group comprising Ringer's lactate, glucose solution, electrolytic solutions, isotonic sodium chloride solution and wherein said infusion solution is present in a volume of 10 ml to 200 ml, preferably 20 ml to 180 ml, more preferably 30 ml to 150 ml and most preferred to 50 ml to 100 ml.

The route of antibody administration is well known in the art and may include, for example, injection or infusion by intravenous, intraperitoneal, intracerebral, intramuscular, intraocular, intraarterial, or intralesional routes, or by sustained release systems. Sustained-release parenteral injections can be divided into several types: oil-based injectable solutions, injectable-drug suspensions, polymer-based microspheres and polymer-based in-situ formings. The antibody can be administered continuously by infusion or by bolus injection. Physiological acceptable solutions are supplemental fluids used in e.g., intravenous therapy to restore or maintain normal fluid volume and electrolyte balance. A person skilled in the art would know that different physiological acceptable solution exists and that the different physiological acceptable solution can be categorized e.g., by their tonicity or purpose. Typically, physiological acceptable solution's tonicity can either be isotonic, hypotonic or hypertonic. In an embodiment of the present invention, the physiological acceptable solution can be selected from the group comprising Ringer's lactate, glucose solution, electrolytic solutions, isotonic sodium chloride solution. In another embodiment of the invention the physiological acceptable solution are also suitable with commercially available infusion or injection carrier solutions for supplying electrolytes without carbohydrate such as isotonic NaCl solution, isotonic glucose solution, Ringer's lactate and similar (Rote Liste 2004, List of finished medicinal products of the members of the Federation of the Pharmaceutical Industry eV, Editio Cantor, Aulendorf/Wuertt., main groups 52.1 and 52.2.1) to the desired concentration or dose, without exhibiting physical or chemical incompatibilities.

Subject matter of the present invention is devices comprising above formulations.

The aqueous pharmaceutical composition according to the present invention can be stored in devices such as a medical container. A medical container according to the present invention is any container suitable for storing the aqueous pharmaceutical composition. Typical medical containers according to the present invention are made of glass or plastic and can be selected from the group comprising vials, prefilled syringes, and cartridges. Said medical container may have a different set of closures: rubber stoppers for vials; plunger, needle, and needle shield (or luer tip and cap) for prefilled syringes; and plunger and seal for cartridges. The person skilled in the art would know how to select a suitable medical container.

Subject matter of the present invention is a ready-for-application aqueous pharmaceutical formulation obtainable by a method according to the present invention comprising said human or humanized anti-adrenomedullin (ADM) antibody or an anti-adrenomedullin antibody fragment in a dose of 1 to 10 mg/kg body weight.

Subject matter of the present invention is a ready-for-application aqueous pharmaceutical formulation obtainable by a method according to the present invention comprising said human or humanized anti-adrenomedullin (ADM) antibody or an anti-adrenomedullin antibody fragment in a dose of 2 to 8 mg/kg body weight, preferred 2 or 4 or 8 mg/kg body weight.

In embodiments of the present invention, ready-for application aqueous pharmaceutical formulation comprising an human or humanized anti-adrenomedullin (ADM) antibody or an anti-adrenomedullin antibody fragment wherein said antibody or said fragment is present in a concentration of at least 0.5 mg/Kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight, or from 2.0 to 5.0 mg/kg body weight.

Subject matter of the present invention is a pharmaceutical formulation for use in therapy or prevention of an acute disease or condition selected from the group comprising: SIRS, a severe infection, sepsis, shock e.g. septic shock, acute vascular diseases as e.g. heart failure, congestion, in particular diuretic resistant congestion, inflammatory conditions, autoimmune diseases, metabolic diseases, brain diseases, cardiovascular diseases and drug-induced diseases symptoms of illness or an illness characterized by such symptoms, wherein the symptoms of illness are selected from the group of nausea, headache, muscle aches, back pain, shivering, vomiting and/or migraine.

Acute disease or acute conditions may be selected from the group but are not limited to the group comprising severe infections as e.g. meningitis, Systemic inflammatory Response-Syndrome (SIRS), or sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata, poisoning, damages induced by chemotherapy.

The pharmaceutical formulation according to the present invention is for reducing the risk of mortality during sepsis and septic shock, i.e., late phases of sepsis.

Septic shock is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin, or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

In the following clinical criteria for SIRS, sepsis, severe sepsis, septic shock will be defined.

### 1) Systemic Inflammatory Host Response (SIRS) Characterized by at Least Two of the Following Symptoms

patients exhibit hypotension (mean arterial pressure is <65 mm Hg)
elevated serum lactate level being >4 mmol/L
blood glucose >7.7 mmol/L (in absence of diabetes)
central venous pressure is not within the range 8-12 mm Hg
urine output is <0.5 mL×kg⁻¹×hr⁻¹
central venous (superior vena cava) oxygen saturation is <70% or mixed venous <65%
heart rate is >90 beats/min
temperature <36° C. or >38° C.
respiratory rate >20/min
white cell count <4 or >12×10⁹/L (leucocytes); >10% immature neutrophils

### 2) Sepsis

Following at least two of the symptoms mentioned under 1), and additionally a clinical suspicion of new infection, being:
cough/sputum/chest pain
abdominal pain/distension/diarrhea
line infection
endocarditis
dysuria
headache with neck stiffness
cellulitis/wound/joint infection
positive microbiology for any infection

### 3) Severe Sepsis

Provided that sepsis is manifested in patient, and additionally a clinical suspicion of any organ dysfunction, being:
blood pressure systolic <90/mean; <65 mmHG
lactate >2 mmol/L
Bilirubin >34 μmol/L
urine output <0.5 mL/kg/h for 2 h
creatinine >177 μmol/L
platelets <100×10⁹/L
SpO₂>90% unless 02 given

### 4) Septic Shock

At least one sign of end-organ dysfunction as mentioned under 3) is manifested.

Septic shock is indicated, if there is refractory hypotension that does not respond to treatment and intravenous systemic fluid administration alone is insufficient to maintain a patient's blood pressure from becoming hypotensive also provides for an administration of a pharmaceutical formulation in accordance with the present invention.

In one specific embodiment of the invention the pharmaceutical formulation is provided for use in therapy of an acute disease or acute condition and wherein the above acute disease or acute condition may be headache, preferably a primary headache, most preferred migraine.

Thus, acute disease or acute conditions may be selected from the group but are not limited to the group comprising severe infections as e.g. meningitis, Systemic inflammatory Response-Syndrome (SIRS), or sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata, poisoning, damages induced by chemotherapy.

In one embodiment of the present invention the patient is not suffering from SIRS, a severe infection, sepsis, shock as e.g., septic shock. Said severe infection denotes e.g., meningitis, Systemic inflammatory Response-Syndrome (SIRS), sepsis, severe sepsis, and shock as e.g., septic shock. In this regard, a severe sepsis is characterized in that sepsis is manifested in said patient, and additionally a clinical suspicion of any organ dysfunction is present, being it:
blood pressure systolic <90/mean; <65 mmHG
lactate >2 mmol/L
Bilirubin >34 μmol/L
urine output <0.5 mL/kg/h for 2h
creatinine >177 μmol/L
platelets <100×10⁹/L
SpO₂>90% unless 02 given

As used herein, the term “nausea” refers to a sensation of unease and discomfort in the upper stomach with an involuntary urge to vomit (Metz A. 2017. Australian Family Physician Vol. 36 (92: 688-692). It may precede vomiting, but a person can have nausea without vomiting.

When prolonged, it is a debilitating symptom. Nausea is a non-specific symptom, which means that it has many possible causes. Some common causes of nausea are motion sickness, dizziness, migraine, fainting, low blood sugar, gastroenteritis (stomach infection) or food poisoning. Nausea is a side effect of many medications including chemotherapy, or morning sickness in early pregnancy. Nausea may also be caused by anxiety, disgust and depression.

As used herein, the term “headache” is the symptom of pain anywhere in the region of the head or neck. It occurs in migraines (sharp or throbbing pains), tension-type headaches, and cluster headaches (Waldman et al. 2014. J Yoga Phys Ther 2014. 4:1). There is also an increased risk of depression in those with severe headaches. Headaches can occur as a result of many conditions whether serious or not. There are a number of different classification systems for headaches. It is well-recognized that causes of headaches may include fatigue, sleep deprivation, stress, effects of medications, the effects of recreational drugs, Viral infections, loud noises, common colds, head injury, rapid ingestion of a very cold food or beverage, and dental or sinus issues.

Headaches are broadly classified as “primary” or “secondary” (Oleson 2005. Functional Neurology 20(2): 61-68). Primary headaches are benign, recurrent headaches not caused by underlying disease or structural problems. For example, migraine is a type of primary headache. While primary headaches may cause significant daily pain and disability, they are not dangerous. The four categories of primary headaches are: migraine, tension-type headache (TTH) cluster headache and other trigeminal autonomic cephalalgias, and other primary headaches.

Secondary headaches, which are of organic, metabolic or drug-‘induced origin, are caused by an underlying disease, like an infection, head injury, vascular disorders, brain bleed or tumors. Secondary headaches can be harmless or dangerous. A migraine is a primary headache disorder characterized by recurrent headaches that are moderate to severe (for review see: Diener et al. 2012. Nat Rev Neurol. 8(3):162-71). Typically, the headaches affect one half of the head, are pulsating in nature, and last from two to 72 hours. Associated symptoms may include nausea, vomiting, and sensitivity to light, sound, or smell. The pain is generally made worse by physical activity. Up to one-third of people have an aura: typically, a short period of visual disturbance, which signals that the headache will soon occur. Occasionally, an aura can occur with little or no headache following it.

As used herein, the terms “muscle aches” or “muscle pain”, also termed “myalgia”, refer to a symptom of many diseases and disorders (for review see: Kyriakides et al. 2013. European Journal of Neurology 20: 997-1005).

The most common causes are the overuse or over-stretching of a muscle or group of muscles. Myalgia without a traumatic history is often due to Viral infections. Longer-term myalgias may be indicative of a metabolic myopathy, some nutritional deficiencies or chronic fatigue syndrome.

As used herein, the term “back pain” refers to painful sensations in the any part of the back. Episodes of back pain may be acute, sub-acute, or chronic depending on the duration. The pain may be characterized as a dull ache, shooting or piercing pain, or a burning sensation. The pain may radiate into the arms and hands as well as the legs or feet and may include paresthesia (tingling with no apparent cause), weakness or numbness in the legs and arms. The anatomic classification of back pain follows the segments of the spine: neck pain (cervical), middle back pain (thoracic), lower back pain (lumbar) or coccydynia (tailbone or sacral pain) with the lumbar vertebrae area most common for pain. The pain may originate from the muscles, nerves, bones, joints or other structures in the vertebral column (spine), however, internal structures such as the gallbladder and pancreas may also cause referred pain in the back (Cohen et al. 2008. BMJ. 33 7.′ü2 718).

As used herein, the term “shivering” (also called “shuddering”) is a bodily function in response to early hypothermia or just feeling cold in warm-blooded animals. When the core body temperature drops, the shivering reflex is triggered to maintain homeostasis. Skeletal muscles begin to shake in small movements, creating warmth by expending energy. Shivering can also be a response to a fever, as a person may feel cold. During fever the hypothalamic set point for temperature is raised. The increased set point causes the body temperature to rise (pyrexia), but also makes the patient feel cold until the new set point is reached. Severe chills with violent shivering are called rigors. Rigors occur because the patient's body is shivering in a physiological attempt to increase body temperature to the new set point. Shivering can also appear after surgery, known as post-anesthetic shivering.

As used herein, the term “vomiting”, also known as emesis and throwing up, among other terms, is the involuntary, forcefi11 expulsion of the contents of one's stomach through the mouth and sometimes the nose (Metz A. 2017. Australian Family physician Vol. 36 (9): 688-692). Vomiting can be caused by a wide variety of conditions; it may present as a specific response to ailments like gastritis or poisoning, or as a non-specific sequela of disorders ranging from brain tumors and elevated intracranial pressure to overexposure to ionizing radiation.

As used herein, the symptoms of illnesses or diseases in a patient in need of therapy and/or prevention of such symptoms are selected from the groups of disease indications comprising inflammatory conditions, autoimmune diseases, metabolic diseases, brain diseases, cardiovascular diseases and drug-induced diseases.

Below, the types of symptoms and illnesses associated herewith are provided in form of non-limiting lists. It is noted that the herein described therapy or prevention may be directed to more than one type of symptom. Further, it is noted that a given medical indication, illness, or disease may be associated with more than one of the symptoms.

The symptom “nausea” may be associated with illnesses inside the abdomen, e. g. obstructing disorders (for example pyloric obstruction, small bowel obstruction, colonic obstruction, superior mesenteric artery syndrome), enteric infections (for example viral or bacterial infection), inflammatory diseases (such as cholecystitis, pancreatitis, appendicitis, hepatitis), sensorimotor dysfunction (for example, gastroparesis, intestinal pseudo-obstruction, gastroesophageal reflux disease, chronic idiopathic nausea, functional vomiting, cyclic vomiting syndrome, rumination syndrome) or biliary colic; illnesses outside the abdomen, e. g. cardiopulmonary disorder (such as cardiomyopathy, myocardial infarction), inner-car diseases (such as motion sickness, labyrinthitis, malignancies), intracerebral disorders (for example hemorrhage, abscess, hydrocephalus, malignancies), psychiatric illnesses (for example anorexia and bulimia nervosa, depression), post-operative vomiting, nausea associated with medications and drugs (for example chemotherapy and biologics therapy, antibiotics, anti-arrhythmics, digoxin, oral hypoglycemic medications, oral contraceptives), nausea associated with endocrine/metabolic diseases (such as pregnancy, uremia, ketoacidosis, thyroid and parathyroid disease, adrenal insufficiency), nausea due to intoxication because of liver failure, alcohol abuse, etc.

The symptom “back pain” may be associated with illnesses due to inflammation, especially in the acute phase, which typically lasts from two weeks to three months, and may be associated with lumbago, trauma, injury, infections, cancer (especially cancers known to spread to the spine like breast, lung and prostate cancer), etc.

The symptom “myalgia” or “muscle pain” may be associated with injury or trauma, including sprains, hematoma, or overuse, wherein a muscle was used too much, too often, including protecting a separate injury, chronic tension, muscle pain due to rhabdomyolysis, associated with, e.g., viral infections, compression injury, drug-related, e.g., due to fibrates and statins, ACE inhibitors, cocaine, some anti-retroviral drugs, severe potassium deficiency, fibromyalgia, Ehlers-Danlos syndrome, auto-immune disorders (including for example mixed connective tissue disease, Systemic lupus erythematosus, polymyalgia rheumatic, Myositis, such as polymyositis, dermatomyositis, and inclusion body myositis, multiple sclerosis, Myalgic Encephalomyelitis (chronic fatigue syndrome), Familial Mediterranean fever, Polyarteritis nodosa, Devic's disease, Morphea, Sarcoidosis), metabolic diseases (such as carnitine palmitoyltransferase II deficiency, Conn's syndrome, Adrenal insufficiency, Hyperthyroidism, Hypothyroidism, Diabetes, Hypogonadism, as well as Channelopathy, Stickler Syndrome, Hypokalemia, Hypotonia (Low Muscle Tone), Exercise intolerance, Mastocytosis, Peripheral neuropathy, Eosinophilia myalgia syndrome, Barcoo Fever, herpes, hemochromatosis also known as iron overload disorder, delayed onset muscle soreness, AIDS, HIV infections, tumor-induced osteomalacia, hypovitaminosis D, myocardial infarction.

The symptom “headache” may be associated with primary headaches. 90% of all headaches are primary headaches. Primary headaches usually first start when people are between 20 and 40 years old. The most common types of primary headaches are migraines and tension-type headaches. They have different characteristics. Migraines typically present with pulsing head pain, nausea, photophobia (sensitivity to light) and phonophobia (sensitivity to sound). Tension-type headaches usually present with non-pulsing “bandlike” pressure on both sides of the head, not accompanied by other symptoms. Other very rare types of primary headaches include cluster headaches: short episodes (15-180 minutes) of severe pain, usually around one eye, with autonomic symptoms (tearing, red eye, nasal congestion) which occur at the same time every day. Cluster headaches can be treated with triptans and prevented with prednisone, ergotamine or lithium; trigeminal neuralgia or occipital neuralgia characterized by shooting face pain, hemicrania continua, i.e., continuous unilateral pain with episodes of severe pain; primary stabbing headaches, e.g., recurrent episodes of stabbing “ice pick pain” or “jabs and jolts” for 1 second to several minutes without autonomic symptoms (tearing, red eye, nasal congestion); Primary cough headache that starts suddenly and lasts for several minutes after coughing, sneezing or straining (anything that may increase pressure in the head). Serious causes (see secondary headaches red flag section) must be ruled out before a diagnosis of “benign” primary cough headache can be made; primary exertional headache characterized by throbbing, pulsatile pain which starts during or after exercising, lasting for 5 minutes to 24 hours. The mechanism behind these headaches is unclear, possibly due to straining causing veins in the head to dilate, causing pain; primary sex headache characterized by a dull, bilateral headache that starts during sexual activity and becomes much worse during orgasm; hypnic headache; secondary headaches that may be caused by problems elsewhere in the head or neck. Some of these are not harmful, such as cervicogenic headache (pain arising from the neck muscles), medication overuse headache in those using excessive painkillers for headaches, meningitis characterized by inflammation of the meninges which presents with fever and meningism's, or stiff neck; bleeding inside the brain (intracranial hemorrhage); subarachnoid hemorrhage (acute, severe headache, stiff neck without fever); ruptured aneurysm, arteriovenous malformation, intraparenchymal hemorrhage; temporal arteritis, i.e., an inflammatory disease of arteries common in the elderly (average age 70), polymyalgia rheumatic; acute closed angle glaucoma (increased pressure in the eyeball); post-ictal headaches that happen after a convulsion or other type of seizure, as part of the period after the seizure (the post-ictal state); gastrointestinal disorders may cause headaches, including Helicobacter pylori infection, celiac disease, non-celiac gluten sensitivity, irritable bowel syndrome, inflammatory bowel disease, gastroparesis, and hepatobiliary disorders. The treatment of the gastrointestinal disorders may lead to a remission or improvement of headaches. The term headache is defined as primary or secondary headache. Primary headache is defined as migraine, tension-type headache (TTH), cluster headache and other trigeminal autonomic cephalalgias, and other primary headaches. Diagnosis or assessment of headache is well-established in the art. Assessment may be performed based on subjective measures, such as patient characterization of symptoms. For example, migraine may be diagnosed based on the following criteria: 1) episodic attacks of headache lasting 4 to 72 hours; 2) with two of the following symptoms: unilateral pain, throbbing, aggravation on movement, and pain of moderate or severe intensity; and 3) one of the following symptoms: nausea or vomiting, and photophobia or phonophobia (Goadsbv et al., N. Engl. J. Med. 346:257-270, 2002).

The symptom “shivering” may be associated with fever, cold sensitivity, menopause, panic attack, anxiety, bacterial infection, rickettsial infection, viral infection, drug withdrawal, etc.

The symptom “vomiting” may be associated with gastritis (inflammation of the gastric wall), gastroenteritis, gastroesophageal reflux disease, celiac disease, non-celiac gluten sensitivity, pyloric stenosis, bowel obstruction, overeating, acute abdomen and/or peritonitis, ileus, food allergies (often in conjunction with hives or swelling); cholecystitis, pancreatitis, appendicitis, hepatitis; food poisoning, allergic reaction to cow's milk proteins, e.g., milk allergy or lactose intolerance; motion sickness, Ménière's disease, concussion, cerebral hemorrhage, migraine, brain tumors, benign intracranial hypertension and hydrocephalus, metabolic disturbances such as hypercalcemia, Uremia adrenal insufficiency, hypoglycemia, hyperglycemia, drug reaction, alcohol intoxication, opioid uptake, selective serotonin reuptake inhibitors; use of chemotherapy drugs; gastric inflammation caused by a range of viruses and bacteria, e.g., norovirus, influenza; or psychiatric/behavioral illnesses such as bulimia nervosa and purge disorder.

Another embodiment of the invention relates to an anti-ADM antibody or an anti-ADM antibody fragment or anti-ADM non-Ig scaffold for use in therapy or prevention of symptoms of illness selected from the group of nausea, headache, muscle aches, back pain, shivering, vomiting or for the use in therapy or prevention of illnesses characterized by such symptoms, such as migraine, in a subject in need thereof according to any of the preceding embodiments to be used in combination with known medicaments against nausea.

Another embodiment of the invention relates to an anti-ADM antibody or an anti-ADM antibody fragment or anti-ADM non-Ig scaffold for use in therapy or prevention of symptoms of illness selected from the group of nausea, headache, muscle aches, back pain, shivering, vomiting or for the use in therapy or prevention of illnesses characterized by such symptoms, such as migraine, in a subject in need thereof according to any of the preceding embodiments to be used in combination with known medicaments against headache.

Another embodiment of the invention relates to an anti-ADM antibody or an anti-ADM antibody fragment or anti-ADM non-Ig scaffold for use in therapy or prevention of symptoms of illness selected from the group of nausea, headache, muscle aches, back pain, shivering, vomiting or for the use in therapy or prevention of illnesses characterized by such symptoms, such as migraine, in a subject in need thereof according to any of the preceding embodiments to be used in combination with known medicaments against myalgia.

Another embodiment of the invention relates to an anti-ADM antibody or an anti-ADM antibody fragment or anti-ADM non-Ig scaffold for use in therapy or prevention of symptoms of illness selected from the group of nausea, headache, muscle aches, back pain, shivering, vomiting or for the use in therapy or prevention of illnesses characterized by such symptoms, such as migraine, in a subject in need thereof according to any of the preceding embodiments to be used in combination with known medicaments against shivering.

Another embodiment of the invention relates to an anti-ADM antibody or an anti-ADM antibody fragment or anti-ADM non-Ig scaffold for use in therapy or prevention of symptoms of illness selected from the group of nausea, headache, muscle aches, back pain, shivering, vomiting or for the use in therapy or prevention of illnesses characterized by such symptoms, such as migraine, in a subject in need thereof according to any of the preceding embodiments to be used in combination with known medicaments against vomiting.

Another embodiment of the invention relates to an anti-ADM antibody or an anti-ADM antibody fragment or anti-ADM non-Ig scaffold for use in therapy or prevention of symptoms of illness selected from the group of nausea, headache, muscle aches, back pain, shivering, vomiting or for the use in therapy or prevention of illnesses characterized by such symptoms, such as migraine, in a subject in need thereof according to any of the preceding embodiments to be used in combination with known medicaments against back pain.

Subject matter of the present invention is a pharmaceutical formulation for use in therapy or prevention of an acute disease or acute condition of a patient for prevention or reduction of organ dysfunction or prevention of organ failure in said patient, wherein said acute disease or acute condition is selected from the group comprising as e.g. severe infections, diabetes, cancer, acute and chronic vascular diseases as e.g. heart failure, myocardial infarction, stroke, atherosclerosis, shock and organ dysfunction,. kidney dysfunction, liver dysfunction, burnings, surgery, traumata, poisoning and damages induced by chemotherapy and wherein said disease is not SIRS, sepsis or septic shock.

“Organ dysfunction” denotes a condition or a state of health where an organ does not perform its expected function. “Organ failure” denotes an organ dysfunction to such a degree that normal homeostasis cannot be maintained without external clinical intervention

The patient group(s) addressed by the instant invention can be defined as set out below.

In the following, clinical criteria are mentioned for respective organs that are prone to dysfunction or failure, and thus represent the patient group(s) of having a chronic or acute disease or acute condition in accordance with the invention:

The criteria orientate on the clinical SOFA score.

The SOFA system was created in a consensus meeting of the European Society of Intensive Care Medicine in 1994 and further revised in 1996.

The SOFA is a six-organ dysfunction/failure score measuring multiple organ failure daily. Each organ is graded from 0 (normal) to 4 (the most abnormal), providing a daily score of 0 to 24 points. The objective of the SOFA is to create a simple, reliable, and continuous score for clinical staff.

Sequential assessment of organ dysfunction during the first few days of intensive care unit (ICU) or hospital admission is a good indicator of prognosis. Both the mean and highest SOFA scores are particularly useful predictors of outcome.

| SOFA score | 0 | 1 | 2 | 3 | 4 |
|---|---|---|---|---|---|
| Respiration | >400 | <400 | <300 | <200 | <100 |
| PaO₂/FIO₂ (mm | | 221-301 | 142-220 | 67-141 | <67 |
| Hg) | | | | | |
| SaO₂/FIO₂ | | | | | |
| Coagulation | >150 | <150 | <100 | <50 | <20 |
| Platelets 10³/mm³ | | | | | |
| Liver | <1.2 | 1.2-1.9 | 2.0-5.9 | 6.0-11.9 | >12.0 |
| Bilirubin (mg/dL) | | | | | |
| Cardiovascular^{b} | No | MAP <70 | Dopamine </=5 | Dopamine >5 or | Dopamine >15 or |
| Hypotension | hypotension | | or dobutamine | norepinephrine </=0.1 | norepinephrine >0.1 |
| | | | (any) | | |
| CNS | 15 | 13-14 | 10-12 | 6-9 | <6 |
| Glasgow Coma | | | | | |
| Score | | | | | |
| Renal | <1.2 | 1.2-1.9 | 2.0-3.4 | 3.5-4.9 or <500 | >5.0 or <200 |
| Creatinine | | | | | |
| (mg/dL) | | | | | |
| or urine output | | | | | |
| (mL/d) | | | | | |

MAP, mean arterial pressure; CNS, central nervous system; SaO₂, peripheral arterial oxygen saturation.

^{a}PaO₂/FIO₂ ratio was used preferentially. If not available, the SaO₂/FIO₂ ratio was used; ^{b}vasoactive mediations administered for at least 1 hr (dopamine and norepinephrine μg/kg/min).

### REFERENCES FOR SOFA SCORE

1. Jones A E, Trzeciak S, Kline J A. The Sequential Organ Failure Assessment score for predicting outcome in patients with severe sepsis and evidence of hypoperfusion at the time of emergency department presentation. Crit Care Med. 2009 May; 37(5):1649-54.
2. Ferreira F L, Bota D P, Bross A, M6lot C, Vincent J L. Serial evaluation of the SOFA score to predict outcome in critically ill patients. JAMA. 2001 Oct. 10; 286(14):1754-8.
3. Vincent J L, Moreno R, Takala J, Willatts S, De Mendonga A, Bruining H, Reinhart C K, Suter P M, Thijs L G. The SOFA (Sepsis-related Organ Failure Assessment) score to describe organ dysfunction/failure. On behalf of the Working Group on Sepsis-Related Problems of the European Society of Intensive Care Medicine. Intensive Care Med. 1996 July; 22(7):707-10.

In a specific embodiment the patient group pursuant to the invention is having as lower threshold at least one SOFA score, being it 1 for one the clinical criteria respiration, or liver, or coagulation, or cardiovascular, or CNS, or renal on day of admission to hospital or Intensive Care Unit (ICU). Thus, said patient group is in need of therapeutic intervention pursuant to the invention, and thus in need for prevention or reduction of organ dysfunction or organ failure

In another specific embodiment the patient group pursuant to the invention is having as lower threshold at least two SOFA scores, being it 1 each for the clinical criteria respiration, and/or liver, and/or coagulation, and/or cardiovascular, and/or CNS, and/or renal on day of admission to hospital or Intensive Care Unit (ICU). Thus, said patient group is in need of therapeutic intervention pursuant to the invention, and thus in need for prevention or reduction of organ dysfunction or organ failure.

In another specific embodiment the patient group pursuant to the invention is having as lower threshold at least three SOFA scores, being it 1 each for the clinical criteria respiration, and/or liver, and/or coagulation, and/or cardiovascular, and/or CNS, and/or renal on day of admission to hospital or Intensive Care Unit (ICU). Thus, said patient group is in need of therapeutic intervention pursuant to the invention, and thus in need for prevention or reduction of organ dysfunction or organ failure.

In another specific embodiment the patient group pursuant to the invention is having as lower threshold at least four SOFA scores, being it 1 each for the clinical criteria respiration, and/or liver, and/or coagulation, and/or cardiovascular, and/or CNS, and/or renal on day of admission to hospital or Intensive Care Unit (ICU). Thus, said patient group is in need of therapeutic intervention pursuant to the invention, and thus in need for prevention or reduction of organ dysfunction or organ failure.

### Patient Group—Kidney Dysfunction/Failure

In the following, said clinical criteria denote the patient group(s) for kidney dysfunction/failure:
Patients at risk for kidney dysfunction/failure: GFR decrease >25%, serum creatinine increased 1.5 times or urine production of <0.5 ml/kg/hr for 6 hours
Patients with present kidney injury: GFR decrease >50%, doubling of creatinine or urine production <0.5 ml/kg/hr for 12 hours
Patients with kidney failure: GFR decrease >75%, tripling of creatinine or creatinine >355 μmol/l (with a rise of >44) (>4 mg/dl) or urine output below 0.3 ml/kg/hr for 24 hours
Patients with loss of kidney function: persistent acute kidney injury (AKI) or complete loss of kidney function for more than 4 weeks
end-stage renal disease: complete loss of kidney function for more than 3 months.

### Patient Group—Liver Dysfunction/Failure

The patient group for liver dysfunction/failure is characterized by a lower threshold of Bilirubin of >1.2 mg/dL, preferably >1.9 mg/dL, more preferably >5.9 mg/dL.

The pharmaceutical formulation may be also administered preventively before the patient exhibits any signs of dysfunction or failure of an organ. This might be the case if the patient has a chronic or acute disease or acute condition where dysfunction or failure problems may be expected, e.g. comprising severe infections as e.g. meningitis, Systemic inflammatory Response-Syndrome (SIRS,) sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata, poisoning. The pharmaceutical formulation may be also administered preventively or therapeutically before, or during or after chemotherapy. The same applies for surgeries where ischemic damages may occur to certain organs which may result in dysfunction or failure of an organ. Preventively means before an organ damage occurs and therapeutically means that an organ damage has been already occurred. Especially useful is the antibody or fragment or scaffold according to the present invention for reducing the risk of organ dysfunction or failure during sepsis and septic shock, i.e., late phases of sepsis.

Subject matter of the present invention is a pharmaceutical formulation for use in therapy or prevention of an acute disease or acute condition of a patient for stabilizing the systemic circulation of said patient, wherein said patient suffers from a disease that is selected from the group comprising SIRS, sepsis, diabetes, cancer, acute vascular diseases as e.g., heart failure, shock as e.g., septic shock and organ dysfunction as e.g., kidney dysfunction.

In a further embodiment of the present invention, a pharmaceutical formulation for use in therapy of an acute disease or acute condition of a patient for stabilizing the circulation, in particular the systemic circulation of said patient. In particular, subject matter of the present invention is a pharmaceutical formulation for use in therapy of an acute disease or acute condition of a patient for stabilizing the systemic circulation of said patient wherein said patient is in need of stabilizing the circulation.

Systemic circulation refers to the part of the circulatory system in which the blood leaves the heart, services the body's cells, and then re-enters the heart. Blood leaves through the left ventricle to the aorta, the body's largest artery. The aorta leads to smaller arteries, arterioles, and finally capillaries. Waste and carbon dioxide diffuse out of the cell into the blood, and oxygen in the blood diffuses into the cell. Blood then moves to venous capillaries, and then to the venae cavae: the lower inferior vena cava and the upper superior vena cava, through which the blood re-enters the heart at the right atrium.

Throughout the specification stabilizing the circulation means stabilizing the systemic circulation. The term systemic circulation would not encompass phenomena of microcirculation. Microcirculation is the delivery of fresh blood to the smallest blood vessels, present in the vasculature embedded within organ tissues. This contrasts with macrocirculation, which transport blood to and from the organs. The state of the systemic circulation may be measures by parameters like mean arterial pressure, blood pressure (other parameters see above). A patient in need for stabilizing the circulation may be, thus a patient that exhibits a heart rate of >100 beats/min and or <65 mm Hg mean arterial pressure. If the circulation is stabilized by the administration of an anti-Adrenomedullin (ADM) antibody or by an anti-ADM antibody fragment binding to adrenomedullin or an anti-ADM non-Ig scaffold binding to adrenomedullin, this can be measured and is characterized by an increase of the mean arterial pressure over 65 mm Hg and/or a decrease of heart rate under 100 beats/min.

It should be emphasized that the provided pharmaceutical formulation is intended by the present invention to be applied for sake of stabilizing the systemic circulation, and thus are not necessarily intended for any methods of primary treatment or first line treatment to the acute disease or acute condition itself that has to be considered as underlying disease(s). This means the present invention do not provide for a therapy of healing/curing e.g., cancer, diabetes, meningitis, polytrauma, and the like. Accordingly, the therapy for an acute disease or acute condition of a patient within the scope of the invention is related to any kind of systemic circulatory insufficiency, or poor systemic circulation of the blood as an acute event.

Subject matter of the present invention is a pharmaceutical formulation
a) for use in therapy of an acute disease or acute condition of a patient for stabilizing the systemic circulation of said patient wherein said patient is in need of stabilizing the systemic circulation and exhibits a heart rate of >100 beats/min and/or <65 mm Hg mean arterial pressure and wherein stabilizing the systemic circulation means increasing the mean arterial pressure over 65 mmHg or
b) for preventive use in therapy of an acute disease or acute condition of a patient in order to prevent that the heart rate increases to >100 beats/min and/or mean arterial pressure decreases to <65 mm Hg.

In all of the above-mentioned acute diseases and conditions there might be the need for stabilizing the circulation of a patient by the administration of a pharmaceutical formulation may also be administered preventively in patients having an acute disease or condition in order to prevent that the heart rate increases of >100 beats/min and/or mean arterial pressure decreases to <65 mm Hg.

In one embodiment the pharmaceutical formulation according to the present invention reduces the vasopressor-agents requirement, e.g., catecholamine requirement, of said patient. The vasopressor-agents requirement, e.g., catecholamine requirement of a patient is an indicator for the condition of the circulation of said patient. Thus, the pharmaceutical formulation may be administered at a point of time when the patient is in need of a vasopressor agent, e.g., catecholamine.

In one embodiment of the invention said patient is a patient in need of increasing the blood pressure.

A patient in need of stabilizing the circulation may a patient with low cardiac output and/or a low blood pressure (hypotension). This may be a patient with a heart rate >100 beats/min. This may be a patient with mean arterial pressures <65 mmHg or even with <60 mmHg. Mean arterial pressure is defined as MAP=(COxSVR)+CVP where CO is cardiac output; SVR is systemic vascular resistance and CVP is central venous pressure and usually small enough to be neglected in this formula. A patient in need of stabilizing the circulation may be also a patient having in addition to the above symptoms a respiratory rate >20/min.

In a specific embodiment of the invention a patient in need of stabilizing the circulation may be a patient with low cardiac output and/or a low blood pressure (hypotension).

This may be a patient with a heart rate >90 beats/min. This may be a patient with mean arterial pressures <65 mmHg or even with <60 mmHg.

Some patients with sepsis-induced hypofusion may remain hypotensive despite adequate fluid replacement. In these cases, vasopressor agents are needed to increase MAP. Thus, in one embodiment of the invention the patient having a chronic or acute disease or acute condition is a patient in need of vasopressor agents to increase MAP. Catecholamines such as dopamine, epinephrine (adrenaline), norepinephrine (noradrenaline), and phenylephrine have been traditionally used to raise blood pressure in patients with e.g., septic shock. Recently also vasopressin has been suggested as potential vasopressor in patients with a chronic or acute disease or acute condition in need for stabilizing the circulation.

Vasopressor agents as catecholamine may stabilize the circulation of a patient having a chronic or acute disease or acute condition. In case the condition of the patient (low blood pressure) is very critical, vasopressor agents' administration, e.g., catecholamine administration, alone may not prevent the break-down of the circulation. The additional administration of said pharmaceutical formulation together with administration of e.g., catecholamine may help to stabilize the circulation of a patient whose condition is so critical that catecholamine administration without administration of said pharmaceutical formulation would not be sufficient in order to stabilize the circulation of said patient.

Further, vasopressors may have serious side effects. Dopamine stimulates D1 receptors in the renal regional circulation, producing vasodilation and increases blood flow. This is one of the reasons why clinicians have utilized low doses of dopamine to protect kidney function. Also, for other vasopressors it has been suggested that increasing the blood pressure with certain drugs, despite its intuitive appeal as something beneficial, can be associated with worse outcomes.

Thus, subject of the invention is a pharmaceutical formulation for use in therapy of an acute disease or acute condition of a patient in order to replace the administration of a vasopressor totally or partially. This means the patient according to the present invention may be a patient being in need of or treatment with vasopressors or a patient receiving a treatment with vasopressors.

The pharmaceutical formulation may be also administered preventively before the patient exhibits any signs of serious circulation problems. This might be the case if the patient has a chronic or acute disease or acute condition where circulation problems may be expected, comprising severe infections as e.g. meningitis, Systemic inflammatory Response-Syndrome (SIRS), or sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata, poisoning, damages induced by chemotherapy.

Especially useful is the antibody or fragment or scaffold according to the present invention for reducing the risk of mortality during sepsis and septic shock, i.e., late phases of sepsis. The person skilled in the art is aware that said reducing the risk of mortality is associated with the stabilization of the circulation in accordance with the invention. Acute disease or acute condition may be a disease or condition wherein the patient is characterized as being in need of stabilizing the circulation. The need of stabilizing the circulation is characterized as outlined above, namely this may be a patient preferably with a heart rate of >90 beats/min or even with a heart rate of >100 beats/min. This may be a patient with mean arterial pressures <65 mmHg or even with <60 mmHg. Mean arterial pressure is defined as MAP=(COxSVR)+CVP where CO is cardiac output; SVR is systemic vascular resistance and CVP is central venous pressure and usually small enough to be neglected in this formula. A patient in need of stabilizing the circulation may be also a patient having a respiratory rate >20/min.

In some embodiments of the invention is also a pharmaceutical formulation to be used in combination with another active drug, e.g., used as primary medicament, wherein said combination is for use in therapy or prevention of a chronic or acute disease or acute condition of a patient for stabilizing the circulation of said patient, in particular the systemic circulation of said patient.

In one embodiment of the invention said patient having a chronic or acute disease or acute condition being in need for stabilizing the circulation is characterized by the need of the patient to get intravenous fluids.

Subject matter of the invention in one specific embodiment is, thus, said pharmaceutical formulation in combination with ADM binding protein and/or further active ingredients for use in therapy or prevention of a patient in need of intravenous fluids. This is in the sense of the patient is in need of intravenous fluids for regulating the systemic fluid balance

Said pharmaceutical formulation thereof with ADM binding protein and/or further active ingredients may be used in combination with vasopressors e.g., catecholamine and/or with fluids administered intravenously for use in a of a chronic or acute disease or acute condition of a patient for stabilizing the circulation, in particular for stabilizing the systemic circulation.

The antibodies, antibody fragments and combinations of the present invention may be used in therapy or prevention of a chronic or acute disease of a patient:
for the prevention of organ dysfunction or organ failure, especially kidney dysfunction or kidney failure and/or,
for stabilizing the circulation, e.g., for reducing the requirement of vasopressors e.g., catecholamine requirement of said patient and/or,
for regulating the fluid balance in said patient.
for reducing the mortality risk for said patient.

Subject matter of the present invention is a pharmaceutical formulation for use in therapy or prevention of an acute disease or acute condition of a patient for the regulation of fluid balance, wherein said patient is a patient in need of regulating the fluid balance and suffers from a disease that is selected from the group comprising systemic inflammatory Response-Syndrome (SIRS), sepsis, diabetes, cancer, heart failure, shock and kidney dysfunction.

The expression “regulating fluid balance” with the context of the instant invention is directed to any correction of a manifested—imbalance—of a patient's fluid balance due to an underlying chronic or acute disease or acute condition. Said correction is in favour of re-establishing normotension in said patients. The person skilled in the art is fully aware that blood pressure in general, as well as hyper- and hypotension is closely related to the fluid balance of a patient.

Fluid balance is the balance of the input and the output of fluids in the body to allow metabolic processes to function. Dehydration is defined as a 1% or greater loss of body mass as a result of fluid loss.

The three elements for assessing fluid balance and hydration status are: clinical assessment, body weight and urine output; review fluid balance charts and review of blood chemistry.

All this is very well known to a person skilled in the art (Alison Shepherd, Nursing Tomes 19.07.11/Vol 107 No 28, pages 12 to 16).

Thus, in one embodiment a person in need of regulating the fluid balance and/or improving the fluid balance of such patients is a person that has a 1% or greater loss of body mass as a result of fluid loss. The fluid balance may be assessed according to Scales and Pilsworth (2008) Nursing Standard 22:47, 50-57. For instance, normal urine output is in the range of 0.5 to 2 ml/kg of body weight per hour. The minimum acceptable urine output for a patient with normal renal function is 0.5 ml/kg per hour. All these standards may be used to assess whether a patient is in need for regulating the fluid balance and/or improving the fluid balance.

Patient in status of fluid imbalance may get fluid administered intravenously as a standard measure of care, especially in an ICU setting. It is, however, desirable to reduce or avoid the additional fluid administration because of complications that might occur as e.g., the occurrence of edema (acroedema). Edema means swelling caused by fluid in the body's tissues. It may occur in feet and legs but can involve the entire body and can involve organs as e.g., lung, heart, eye. Thus, said pharmaceutical formulation may be administered at a point of time when the patient is in need of fluid administration. According to the invention said patient is a patient in need of regulating the fluid balance.

Thus, subject matter of the present invention is also a pharmaceutical formulation for use in therapy of an acute disease or acute condition of a patient for the regulation of fluid balance which includes but is not limited to the prevention or reduction of edema.

### Fluid Balance/Fluid Therapy

In an acute hospital setting, being it e.g., a setting in the ICU, commonly the fluid balance is monitored carefully by the clinical staff since this provides for particular information on a patient's actual state of hydration, and thus for renal and cardiovascular function.

If, however, acute fluid loss is greater than fluid gain, the patient is referred to as being in negative fluid balance. In this case, physiological fluid is often given intravenously by a physician to compensate for that loss.

In contrast, a positive fluid balance where fluid gain is greater than fluid loss may provide for information to a problem with either the renal or cardiovascular system.

This particularly means in context with e.g. SIRS, sepsis, severe sepsis and septic shock, that also blood pressure is low (commonly referred to as hypotension), and the filtration rate in the kidneys will lessen, thus causing less fluid reabsorption and less urine output.

The term “fluid therapy” in general denotes the therapeutic administration of fluids (such as physiologic saline solution or water for injection (WFI)) to a patient as a treatment or preventative measure. It can be administered via intravenous, intraperitoneal, intraosseous, subcutaneous, and oral routes.

Fluid therapy is indicated either when there is a loss of fluid or there is a risk of loss of fluid due to an underlying disease or condition. The severity of the fluid loss, and the compartment from which it has been lost, influences the choice of fluid and the speed at which it needs to be administered. If fluid therapy is performed as a treatment, then it is necessary to diagnose and treat the underlying disease or condition. Fluid therapy is routinely indicated in case of hypotension, hypovolemia, metabolic disorders, decreased oxygen delivery, SIRS, sepsis, severe sepsis, shock, and septic shock.

However, it should be emphasized that the medicaments provided by the present invention, being said pharmaceutical formulation are only intended to be used for sake of regulating the fluid balance and thus not for any methods of primary treatment to a chronic or acute disease or condition itself. This means the present invention does not provide for a therapy of healing/curing e.g. meningitis, Systemic inflammatory Response-Syndrome (SIRS), or sepsis, or severe sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata, poisoning, or damages induced by chemotherapy within the scope of the invention.

The fluid regulating effect of the pharmaceutical formulation is thus supporting the primary therapy of said chronic or acute disease or acute condition. In case of a chronic or acute disease or acute condition like severe infections as e.g., meningitis, Systemic inflammatory Response-Syndrome (SIRS), sepsis or the like the primary therapy would be e.g., the administration of antibiotics. The pharmaceutical formulation would regulate the fluid balance and would help to prevent worsening of the critical condition of said patient until the e.g., antibiotic administration takes effect. As mentioned before, the pharmaceutical formulation may be administered in a preventive way or in a therapeutic way, this means in order to prevent fluid imbalance problems or in order to reduce fluid imbalance when fluid imbalance problems are present in said patient. Edema is included in the term fluid imbalance problems.

In one embodiment of the invention said patient having a chronic or acute disease or condition being in need for regulation of fluid balance is characterized by the need of said patient to get vasopressor agents, e.g., catecholamine, administration.

Subject matter of the invention in one specific embodiment is, thus, said pharmaceutical formulation for use in therapy of a patient in need of a vasopressor agent, e.g., catecholamine treatment.

A patient in need of improvement of fluid balance may be characterized by a capillary leakage and may be a urine output </=0.5-1 cc/kg per hour.

Subject matter of the present invention is a pharmaceutical formulation for use in therapy or prevention of congestion in a patient wherein said patient has a disease or condition selected from the group comprising: congestive high blood pressure, swelling or water retention (edema), heart failure in particular acute heart failure, kidney or liver disease.

In a specific embodiment said pharmaceutical formulation may be administered to a patient with vascular barrier dysfunction or endothelial dysfunction that may result in congestion.

Vascular barrier dysfunction or endothelial dysfunction is a systemic pathological state of the endothelium (the inner lining of blood vessels) and can be broadly defined as an imbalance between vasodilating and vasoconstricting substances produced by (or acting on) the endothelium (Deanfield et al. 2005. J Hypertens 23 (1): 7-17). Normal functions of endothelial cells include mediation of coagulation, platelet adhesion, immune function and control of volume and electrolyte content of the intravascular and extravascular spaces. The endothelium is a cellular monolayer that lines the entire cardiovascular system and regulates many processes including vascular tone, thrombosis, angiogenesis, and inflammation. Endothelial cells have been shown to be phenotypically dynamic and, in response to a variety of local and systemic stimuli, are able to transition between quiescent and activated states (Colombo et al. 2015. Curr Heart Fail Rep. 12(3): 215-222). In recent years, emerging research has demonstrated that endothelial dysfunction is a major contributor to cardiovascular disease, including hypertension, atherosclerosis, and congestive heart failure (Gutierrez et al. 2013. European Heart Journal 34: 3175-3181).

The endothelium tightly controls the exchange of fluid from the circulation to the surrounding tissues and dysfunction of this barrier leads to uncontrolled fluid extravasation that may result in congestion and/or edema. A common feature of edema (e.g., pulmonary edema) is increased permeability to water low molecular weight solutes (Rocker et al. 1987. Thorax 42: 620-23).

Endothelial dysfunction can result from and/or contribute to several disease processes, as occurs in hypertension, hypercholesterolaemia, diabetes or septic shock. Endothelial dysfunction is a major pathophysiological mechanism that leads towards coronary artery disease, and other atherosclerotic diseases.

From preclinical experiments in models of sepsis/septic shock it is known that administration of the anti-ADM antibody induces an increase of the plasma bio-ADM concentration and that this coincides with an increased survival rate (Struck et al. 2013. Intensive Care Med Exp 1(1):22). The mechanism underlying this effect is thought as follows:

The pharmaceutical formulation, when administered i.v., due to its size cannot cross the endothelial barrier into the interstitium but remains in the blood circulation. In contrast, ADM, as a small peptide, can freely diffuse across the endothelial barrier. Thus, the antibody, when administered in a vast molar excess over the endogenous ADM, binds virtually all ADM in the plasma and, as a simple consequence of reaching binding equilibrium, leads to a translocation of ADM from the interstitium to the blood circulation. Interstitially located ADM can bind to vascular smooth muscle cells and induces relaxation resulting in vasodilation. This is reduced by administration of the antibody. On the other hand, ADM in plasma binds to endothelial cells and thereby stabilizes or even restores vascular integrity. Thus, this function is strengthened, when plasma ADM levels increase as a consequence of administration of the antibody, which is a non-neutralizing antibody. Finally, binding of the antibody to ADM reduces the proteolytic decay of ADM.

Surprisingly, we have observed in the PROTECT study and the BIOSTAT study (see e.g., WO2018/109228), that in subjects with heart failure bio-ADM concentrations increase with the presence and severity of congestion, despite their treatment with diuretics. Thus, bio-ADM increase in these patients is the body's counter regulation of the tissue congestion. However, the natural increase is insufficient to effectively achieve this counter regulation. Tissue congestion also occurs in sepsis. It could be demonstrated e.g., in WO2018/109228 that administration of the anti-ADM antibody in septic animal models leads to restoration of impaired vascular integrity. Due to the parallel mechanism of tissue congestion in both sepsis and heart failure, it is credible for the expert in the field, that administration of the anti-ADM antibody must be beneficial in the treatment of congestion in heart failure, similar as in sepsis/septic shock.

In a specific embodiment of the invention said pharmaceutical formulation is for use in intervention and therapy of congestion in a patient according to any embodiment of the invention, wherein said patient is resistant against diuretics or is a non-responder to diuretics therapy.

Another specific embodiment of the invention relates to said pharmaceutical formulation for use in intervention and therapy of congestion in a patient in need thereof and wherein said patient is resistant against diuretics or is a non-responder to diuretics therapy.

The term “diuretic resistance” is defined in general as failure to decrease the extracellular fluid volume despite liberal use of diuretics (Ravnan et al. 2002. CHF 8:80-85). Epstein et al. defined diuretic resistance as a failure to excrete at least 90 mmol of sodium within 72 hours of a 160-mg oral furosemide dose given twice daily (Epstein et al. 1977. Curr Ther Res. 21:656-667).

Adaptation to diuretic drugs and diuretic resistance may be caused by similar mechanisms. Diuretic adaptations can be classified as those that occur during diuretic action, those that cause sodium retention in the short term (causing ‘post-diuretic NaCl retention’), and those that increase sodium retention chronically (the ‘braking phenomenon’). Ways in which kidneys adapt to chronic diuretic treatment are: First, nephron segments downstream from the site of diuretic action increase NaCl reabsorption during diuretic administration because delivered NaCl load is increased. Second, when diuretic concentrations in the tubule decline, the kidney tubules act to retain Na until the next dose of diuretic is administered. Third, the ability of the diuretic to increase renal NaCl excretion declines over time, an effect that results both from depletion of the extracellular fluid volume and from structural and functional changes of kidney tubules themselves. These adaptations all increase the rate of NaCl reabsorption and blunt the effectiveness of diuretic therapy. For review see Ellison 1999. Semin Nephrol. 19(6):581-97 and De Bruyne 2003. Postgrad Med J 79:268-271.

Although difficult to quantify, diuretic resistance is thought to occur in one of three patients with congestive HF. Heart failure represents the most common clinical situation in which diuretic resistance is observed. In mild congestive HF, diuretic resistance is not commonly encountered, as long as renal function is preserved. However, in moderate and severe congestive HF patients, diuretic resistance occurs more frequently and often becomes a clinical problem (Brater 1985. Drugs 30:427-443; Taylor 2000 Cardiol Rev. 8:104-114).

Subject matter of the present invention is a pharmaceutical formulation for use in therapy or prevention comprising:
determining the level of a fragment of pre-pro-Adrenomedullin selected from the group comprising Midregional Proadrenomedullin (MR-proADM), C-terminal Proadrenomedullin (CT-proADM), Bio-ADM, ADM-gly or Proadrenomedullin N-terminal 20 peptide (PAMP) or fragments thereof prior to drug administration in a bodily fluid obtained from said subject; and
comparing said level of a fragment of pre-pro-Adrenomedullin selected from the group comprising MR-proADM, CT-proADM, PAMP, Bio-ADM or ADM-gly to a predefined threshold or to a previously determined level of said fragment, and
wherein for the correlation an elevated level of said fragment of pre-pro-Adrenomedullin selected from the group comprising MR-proADM, CT-proADM, PAMP, Bio-ADM or ADM-gly or fragments thereof above a certain threshold or above a previously determined level is used for patient stratification for a therapeutic use according to the present invention.

As used herein, the term “PAMP” comprises both circulating forms of PAMP, namely a biologically inactive C-terminally Glycine-extended PAMP (PAMP-Gly) and a biologically active C-terminally amidated PAMP (PAMP-amide).

As used herein, the term “Bio-ADM” and “ADM-NH2” are to be used synonymously and both refer to the circulating bioactive Adrenomedullin.

In another embodiment of the present application fragments of pre-pro-Adrenomedullin that may be determined in a bodily fluid is/are selected from the group comprising:

(Proadrenomedullin N-20 terminal peptide, PAMP): amino acids 22-41
of preproADM
SEQ ID No. 10
ARLDVASEFRKKWNKWALSR
(Midregional proAdrenomedullin, MR-proADM): amino acids 45-92
of preproADM
SEQ ID No. 11
ELRMSS SYPTGLADVK AGPAQTLIRP QDMKGASRSP EDSSPDAARI RV
(C-terminal proAdrenomedullin, CT-proADM): amino acids 148-185
of preproADM
SEQ ID No. 12
RRR RRSLPEAGPG RTLVSSKPQA HGAPAPPSGS APHFL
SEQ ID No. 13: (mature human Adrenomedullin (mature ADM); amidated
ADM; bio-ADM; ADM-NH2): amino acids 1-52 or amino acids 95-146
of pro-ADM
YRQSMNNFQGLRSFGCRFGTCTVQKLAHQIYQFTDKDKDNVAPRSKISPQGY-CONH2
(Adrenomedullin 1-52-Gly (ADM 1-52-Gly): amino acids 95-147 of
preproADM)
SEQ ID No.: 14
YRQSMN NFQGLRSFGC RFGTCTVQKL AHQIYQFTDK DKDNVAPRSK ISPQGYG

In another embodiment of the present application said fragment of pre-proAdrenomedullin having at least 5 amino acids is/are selected from the group comprising MR-proADM (SEQ ID No. 11), CT-proADM (SEQ ID No. 12) and/or PAMP (SEQ ID No. 10).

In one embodiment of the present application the level of the fragments of pre-proADM and/or fragments thereof is determined by using at least one binder, wherein said binder binds to a region comprised within the sequence of MR-proADM (SEQ ID No. 11).

In another embodiment of the present application the level of the fragments of pre-pro-ADM and/or fragments thereof is determined by using at least one binder, wherein said binder binds to a region comprised within the sequence of CT-proADM (SEQ ID No. 12).

In another embodiment of the present application the level of the fragments of pre-proADM and/or fragments thereof is determined by using at least one binder, wherein said binder binds to a region comprised within the sequence of PAMP (SEQ ID No. 10).

Subject matter in a particular embodiment of the present application is a method, wherein said fragment may be selected from MR-proADM according to SEQ ID No.: 11 and/or CT-proADM according to SEQ ID No.: 12 and/or PAMP according to SEQ ID No.: 10.

In a specific embodiment of the diagnostic method said proADM and/or fragments thereof having at least 5 amino acids is/are selected from the group comprising mature ADM-NH₂ (SEQ ID No. 13), ADM 1-52-Gly (SEQ ID No. 14), MR-proADM (SEQ ID No. 11) and CT-proADM (SEQ ID No. 12).

In a specific embodiment of the diagnostic method either the level of mature ADM-NH₂ (SEQ ID No. 13) and/or ADM 1-52-Gly (SEQ ID No. 14)—immunoreactivity or the level of MR-proADM (SEQ ID No. 11) immunoreactivity or the level of CT-proADM (SEQ ID No. 12) immunoreactivity is determined and correlated with the need of said patient for therapy or intervention, wherein said patient is identified as having such a need if the level of mature ADM-NH₂ (SEQ ID No. 13) and/or ADM 1-52-Gly (SEQ ID No. 14)—immunoreactivity or the level of MR-proADM (SEQ ID No. 11) immunoreactivity or the level of CT-proADM (SEQ ID No. 12) immunoreactivity in the bodily fluid of said subject is above a threshold.

In a specific embodiment of the diagnostic method the level of proADM and/or fragments thereof is determined by using at least one binder selected from the group: a binder that binds to a region comprised within the following sequence of mature ADM-NH₂ (SEQ ID No. 13) and/or ADM1-52-Gly (SEQ ID No. 14) and a second binder that binds to a region comprised within the sequence of mature ADM-NH₂ (SEQ ID NO. 13) and/or ADM 1-52-Gly (SEQ ID No. 14).

In a specific embodiment of the diagnostic method the level of proADM and/or fragments thereof is determined by using at least one binder selected from the group: a binder that binds to a region comprised within the sequence of MR-proADM (SEQ ID No. 11) and a second binder that binds to a region comprised within the sequence of MR-proADM (SEQ ID No. 11).

In a specific embodiment of the diagnostic method the level of pro-ADM and/or fragments thereof is determined by using at least one binder selected from the group: a binder that binds to a region comprised within the sequence of CT-proADM (SEQ ID No. 12) and a second binder that binds to a region comprised within the sequence of CT-pro-ADM (SEQ ID No. 12).

Subject matter in a particular embodiment of the present diagnostic method is a method, according to the present invention wherein said fragment may be selected from MR-proADM according to SEQ ID No.: 11 or mature ADM-NH₂ according to SEQ ID No.: 13.

In a specific embodiment of the invention the threshold is within a threshold range for plasma MR-proADM that is between 0.5 and 1.5 nmol/L, preferably between 0.7 and 1 nmol/L, most preferred a threshold of 0.8 nmol/L is applied.

In a specific embodiment of the invention the predetermined threshold of Bio-ADM in a sample of bodily fluid of said subject is between 40 and 100 μg/mL, more preferred between 50 and 90 μg/mL, even more preferred between 60 and 80 μg/mL, most preferred said threshold is 70 μg/mL.

In a specific embodiment of the invention the threshold is within a threshold range for plasma CT-proADM that is between 85 and 350 μmol/L, preferably between 100 and 250 μmol/L, most preferred a threshold of 150 μmol/L is applied.

In a specific embodiment of the invention a threshold for plasma PAMP-amide that is between 0.3 and 1.2 μmol/L, preferably between 0.4 and 1.0 μmol/L, most preferred a threshold of 0.8 μmol/L is applied.

In a specific embodiment of the invention a threshold for plasma PAMP-glycine that is between 0.5 and 2.0 μmol/L, preferably between 0.7 and 1.8 μmol/L, most preferred a threshold of 1.5 μmol/L is applied.

In a specific embodiment of the invention the threshold is within a threshold range for plasma ADM-NH₂ that is between 50 and 100 μg/ml, preferably between 60 and 90 μg/ml, most preferred a threshold of 70 μg/ml is applied.

In a specific embodiment of the invention a threshold for plasma Bio-ADM is the 5fold median concentration, preferably the 4fold median concentration, more preferred the 3fold median concentration, most preferred the 2fold median concentration of a normal healthy population.

In a specific embodiment of the invention a threshold for plasma MR-proADM is the 5fold median concentration, preferably the 4fold median concentration, more preferred the 3fold median concentration, most preferred the 2fold median concentration of a normal healthy population.

In a specific embodiment of the invention a threshold for plasma CT-proADM is the 5fold median concentration, preferably the 4fold median concentration, more preferred the 3fold median concentration, most preferred the 2fold median concentration of a normal healthy population.

In a specific embodiment of the invention a threshold for plasma ADM-Gly is the 5fold median concentration, preferably the 4fold median concentration, more preferred the 3fold median concentration, most preferred the 2fold median concentration of a normal healthy population.

In a specific embodiment of the invention a threshold for plasma PAMP is the 5fold median concentration, preferably the 4fold median concentration, more preferred the 3fold median concentration, most preferred the 2fold median concentration of a normal healthy population.

Threshold levels can be obtained for instance from a Kaplan-Meier analysis, where the occurrence of a disease is correlated with the quartiles of the biomarker in the population. According to this analysis, subjects with biomarker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

The above-mentioned threshold values might be different in other assays, if these have been calibrated differently from the assay system used in the present invention. Therefore, the above-mentioned threshold shall apply for such differently calibrated assays, accordingly, taking into account the differences in calibration. One possibility of quantifying the difference in calibration is a method comparison analysis (correlation) of the assay in question (e.g., bio-ADM assay) with the respective biomarker assay used in the present invention by measuring the respective biomarker (e.g., bio-ADM) in samples using both methods. Another possibility is to determine with the assay in question, given this test has sufficient analytical sensitivity, the median biomarker level of a representative normal population, compare results with the median biomarker levels as described in the literature (e.g., Weber et al. 2017. JALM 2(2): 222-233) and recalculate the calibration based on the difference obtained by this comparison. With the calibration used in the present invention, samples from normal (healthy) subjects have been measured: median plasma bio-ADM (mature ADM-NH₂) was 13.7 μg/ml (inter quartile range [IQR]9.6-18.7 μg/mL) (Weber et al. 2017. JALM 2(2): 222-233).

Subject matter of the present diagnostic method is a method according to the diagnostic method invention, wherein the level of Pro-Adrenomedullin or fragments thereof of at least 5 amino acids is determined by using a binder to Pro-Adrenomedullin or fragments thereof of at least 5 amino acids.

Subject matter of the present invention is a pharmaceutical formulation for use in therapy or prevention for use in therapy of a patient with shock, in particular septic shock, wherein said patient:
has suffered from shock, in particular from a septic shock not longer than 8.4 hours at the starting point of treatment with said Anti-adrenomedullin (ADM) antibody or an anti-adrenomedullin antibody fragment and/or
has been admitted to ICU not longer than 8.4 hours at the starting point of treatment with said Anti-adrenomedullin (ADM) antibody or an anti-adrenomedullin antibody fragment, and/or
has not received organ support at all or not longer than 8.4 hours of organ support at the starting point of treatment with said Anti-adrenomedullin (ADM) antibody or an anti-adrenomedullin antibody fragment, and wherein said antibody or fragment binds to the N-terminal part (aa 1-21) of ADM:

(SEQ ID NO. 1)
YRQSMNNFQGLRSFGCRFGTC.

As used herein, the term “admission to ICU” refers to patients admitted to intensive care are patients who have, or are likely to have, one or several acute, directly life-threatening malfunctions which require the use of organ supporting methods. Criteria for admitting patients to intensive care units have been developed which are well documented in the art (Nates et al. (2016), Critical care medicine 44: 1553-1602). The term “admission to ICU” shall encompass admissions under these criteria.

Types of organ support include:
Respiratory support therapy:
   comprising advanced respiratory support therapy such as tracheal intubation and mechanical ventilation support
   basic respiratory support therapy such as use of supplemental oxygen, the use of an incentive spirometer, chest percussion nebulization, etc.
Circulatory support therapy, such as mechanical circulatory support (e.g., the use of an intra-aortic balloon pump and a ventricular assist device) and the medical therapy including the use of an angiotensin converting enzyme, beta blockers, etc.
Renal support therapy such as hemodialysis and peritoneal dialysis,
Hemodynamic monitoring or support therapy such as measuring the pressure, flow and oxygen content of the blood, fluid resuscitation or blood transfusion and the use of vasoactive drugs 8e.g. nitroglycerine, nitric oxide, etc.),
Neurological monitoring or support such as an intraventricular catheter.

The extracorporeal organ support is described in detail for example in ICU Management & Practice, Volume 18-Issue 1, 2018.

In a preferred embodiment, adrenomedullin (ADM) antibody or an anti-adrenomedullin antibody fragment or anti-ADM non-Ig scaffold is for use in therapy of a patient with shock, in particular septic shock, wherein said patient:
has suffered from shock, in particular from a septic shock no longer than 10 hours at the starting point of treatment with said Anti-adrenomedullin (ADM) antibody or an anti-adrenomedullin antibody fragment or anti-ADM non-Ig scaffold and/or
has been admitted to ICU not longer than 10 hours at the starting point of treatment with said Anti-adrenomedullin (ADM) antibody or an anti-adrenomedullin antibody fragment or anti-ADM non-Ig scaffold, and/or
has not received organ support at all or not longer than 10 hours of organ support at the starting point of treatment with said Anti-adrenomedullin (ADM) antibody or an anti-adrenomedullin antibody fragment or anti-ADM non-Ig scaffold, and wherein said antibody or fragment or scaffold binds to the N-terminal part (aa 1-21) of ADM:

(SEQ ID NO. 1)
YRQSMNNFQGLRSFGCRFGTC

wherein, when the patient has suffered from shock, in particular from a septic shock a) not longer than 10 hours and has been admitted to ICU b) not longer than 10 hours, the starting point of treatment with said Anti-adrenomedullin (ADM) antibody or an anti-adrenomedullin antibody fragment or anti-ADM non-Ig scaffold is the shortest of a) and b).

In another embodiment, when the patient has been admitted to ICU a) not longer than 10 hours and c) has received no longer than 10 hours of organ support, the starting point of treatment with the Anti-adrenomedullin (ADM) antibody or an anti-adrenomedullin antibody fragment or anti-ADM non-Ig scaffold is the shortest of a) and c).

In another embodiment, when the patient has suffered from shock, in particular from a septic shock b) not longer than 10 hours and has received c) no longer than 10 hours of organ support, the starting point of treatment with the Anti-adrenomedullin (ADM) antibody or an anti-adrenomedullin antibody fragment or anti-ADM non-Ig scaffold is the shortest of b) and c).

In another embodiment, when the patient has suffered from shock, in particular from a septic shock a) not longer than 10 hours and the patient has been in shock b) not longer than 10 hours and has received c) no longer than 10 hours of organ support, the starting point of treatment with the Anti-adrenomedullin (ADM) antibody or an anti-adrenomedullin antibody fragment or anti-ADM non-Ig scaffold is the shortest of a), b) and c).

In another embodiment, the invention relates to an adrenomedullin (ADM) antibody or an anti-adrenomedullin antibody fragment or anti-ADM non-Ig scaffold for use in therapy of a patient with shock, in particular septic shock, wherein said patient:
has been in shock not longer than 10 hours at the starting point of treatment with said Anti-adrenomedullin (ADM) antibody or an anti-adrenomedullin antibody fragment or anti-ADM non-Ig scaffold and/or
has been admitted to ICU not longer than 10 hours at the starting point of treatment with said Anti-adrenomedullin (ADM) antibody or an anti-adrenomedullin antibody fragment or anti-ADM non-Ig scaffold, and/or
has not received organ support at all or not longer than 10 hours of organ support at the starting point of treatment with said Anti-adrenomedullin (ADM) antibody or an anti-adrenomedullin antibody fragment or anti-ADM non-Ig scaffold, and wherein said antibody or fragment or scaffold binds to the N-terminal part (aa 1-21) of ADM:

(SEQ ID NO. 1)
YRQSMNNFQGLRSFGCRFGTC.

In a preferred embodiment, the patient has suffered from shock, in particular from a septic shock, not longer than 9, preferably 8.4, preferably 8.26 (0.344 days), preferably 8, preferably 7, preferably 6, preferably 5.76 (0.25 days), preferably 5.75 (0.24 days), 5, preferably 4, preferably 3 hours at the starting point of treatment with said Anti-adrenomedullin (ADM) antibody or an anti-adrenomedullin antibody fragment or anti-ADM non-Ig scaffold.

In a preferred embodiment, the patient has suffered from shock, in particular from a septic shock, not longer than 8.4, preferably 8.26 (0.344 days) at the starting point of treatment with said Anti-adrenomedullin (ADM) antibody or an anti-adrenomedullin antibody fragment or anti-ADM non-Ig scaffold.

In a preferred embodiment, the patient has been admitted to ICU not longer than 9, preferably 8.4, preferably 8.26 (0.344 days), preferably 8, preferably 7, preferably 6, preferably 5.76 (0.25 days), preferably 5.75 (0.24 days) preferably 5, preferably 4, preferably 3 hours at the starting point of treatment with said Anti-adrenomedullin (ADM) antibody or an anti-adrenomedullin antibody fragment or anti-ADM non-Ig scaffold.

In a preferred embodiment, the patient has been admitted to ICU not longer than 8.4, preferably no longer than 8.26 (0.344 days), at the starting point of treatment with said Anti-adrenomedullin (ADM) antibody or an anti-adrenomedullin antibody fragment or anti-ADM non-Ig scaffold.

In a preferred embodiment, the patient has received organ support not longer than 9, preferably 8.4, preferably 8.26 (0.344 days), preferably 8, preferably 7, preferably 6, preferably 5.76 (0.25 days), preferably 5.75 (0.24 days), preferably 5, preferably 4, preferably 3 hours at the starting point of treatment with said Anti-adrenomedullin (ADM) antibody or an anti-adrenomedullin antibody fragment or anti-ADM non-Ig scaffold.

In a preferred embodiment, the patient has received organ support not longer than 8.4, preferably no longer than 8.26 (0.344 days at the starting point of treatment with said Anti-adrenomedullin (ADM) antibody or an anti-adrenomedullin antibody fragment or anti-ADM non-Ig scaffold

In another embodiment, the anti-Adrenomedullin—adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or not longer than 10h of organ support, and
wherein said antibody or fragment or scaffold binds to the N-terminal part (aa 1-21) of ADM:

(SEQ ID NO. 1)
YRQSMNNFQGLRSFGCRFGTC.

In another embodiment, the anti-Adrenomedullin—adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 8.4 hours after occurrence of shock in said patient and/or
within 8.4 hours after admission of said patient to ICU, and/or
before the patient has received organ support or not longer than 10h of organ support, and
wherein said antibody or fragment or scaffold binds to the N-terminal part (aa 1-21) of ADM:

(SEQ ID NO. 1)
YRQSMNNFQGLRSFGCRFGTC.

In preferred embodiment, the anti-Adrenomedullin-adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or not longer than 10 h of organ support, and
wherein said antibody or fragment or scaffold binds to the N-terminal part (aa 1-21) of ADM:

(SEQ ID No. 1)
YRQSMNNFQGLRSFGCRFGTC,

wherein, when the Anti-adrenomedullin (ADM) antibody or an anti-adrenomedullin antibody fragment or anti-ADM non-Ig scaffold can be administered a) within 10 hours after occurrence of shock in said patient and b) within 10 hours after admission of said patient to ICU, the Anti-adrenomedullin (ADM) antibody or an anti-adrenomedullin antibody fragment or anti-ADM non-Ig scaffold is administered at the shortest of a) and b).

In another embodiment, when the Anti-adrenomedullin (ADM) antibody or an anti-adrenomedullin antibody fragment or anti-ADM non-Ig scaffold can be administered a) within 10 hours after occurrence of shock in said patient and c) before the patient has received organ support or not longer than 10h of organ support, the Anti-adrenomedullin (ADM) antibody or an anti-adrenomedullin antibody fragment or anti-ADM non-Ig scaffold is administered at the shortest of a) and c).

In another embodiment, when the Anti-adrenomedullin (ADM) antibody or an anti-adrenomedullin antibody fragment or anti-ADM non-Ig scaffold can be administered b) within 10 hours after admission of said patient to ICU and c) before the patient has received organ support or not longer than 10h of organ support, the Anti-adrenomedullin (ADM) antibody or an anti-adrenomedullin antibody fragment or anti-ADM non-Ig scaffold is administered at the shortest of b) and c).

In another embodiment, when the Anti-adrenomedullin (ADM) antibody or an anti-adrenomedullin antibody fragment or anti-ADM non-Ig scaffold can be administered a) within 10 hours after occurrence of shock in said patient and b) within 10 hours after admission of said patient to ICU and c) before the patient has received organ support or not longer than 10 hours of organ support, the Anti-adrenomedullin (ADM) antibody or an anti-adrenomedullin antibody fragment or anti-ADM non-Ig scaffold is administered at the shortest of a), b) and c).

In another embodiment, the anti-Adrenomedullin-adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or not longer than 10 hours of organ support, and
wherein said antibody or fragment or scaffold binds to the N-terminal part (aa 1-21) of ADM:

(SEQ ID NO. 1)
YRQSMNNFQGLRSFGCRFGTC.

In a preferred embodiment, the Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5, preferably 4, preferably 3 hours after occurrence of shock and/or sepsis in said patient.

In a preferred embodiment, the Anti-adrenomedullin (ADM) antibody or an anti-adrenomedullin antibody fragment or anti-ADM non-Ig scaffold is administered within 8.4, preferably 8.26 (0.344 days) hours after occurrence of shock and/or sepsis in said patient.

In a preferred embodiment, the Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5, preferably 4, preferably 3 hours after admission of said patient to ICU.

In a preferred embodiment, the Anti-adrenomedullin (ADM) antibody or an anti-adrenomedullin antibody fragment or anti-ADM non-Ig scaffold is administered 8.4, preferably 8.26 (0.344 days) hours after admission of said patient to ICU.

In a preferred embodiment, the Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5, preferably 4, preferably 3 hours after the patient has received organ support at the starting point of treatment with said Anti-adrenomedullin (ADM) antibody or an anti-adrenomedullin antibody fragment or anti-ADM non-Ig scaffold.

In a preferred embodiment, the Anti-adrenomedullin (ADM) antibody or an anti-adrenomedullin antibody fragment or anti-ADM non-Ig scaffold is administered 8.4, preferably 8.26 (0.344 days) hours after the patient has received organ support at the starting point of treatment with said Anti-adrenomedullin (ADM) antibody or an anti-adrenomedullin antibody fragment or anti-ADM non-Ig scaffold.

In another specific embodiment of the invention, said shock is selected from the group comprising shock due to hypovolemia, cardiogenic shock, obstructive shock and distributive shock, in particular cardiogenic or septic shock.

In a specific embodiment of the invention, said shock is selected from the group comprising:
in case of cardiogenic shock said patient has suffered an acute coronary syndrome (e.g., acute myocardial infarction) or has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient has septic shock, neurogenic shock, anaphylactic shock or shock due to adrenal crisis.

In a more preferred embodiment, the shock is a septic shock, shock due to Covid-19, shock due to burns or a traumatic shock.

In a further embodiment, the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, wherein a sample of bodily fluid taken said patient exhibits a level of bioADM>70 μg/mL, and wherein said bodily fluid is selected from the group comprising whole blood, plasma, serum.

Subject matter of the present invention is a pharmaceutical formulation for use in therapy or prevention for use in therapy wherein said patient has shock that is selected from the group comprising shock due to hypovolemia, cardiogenic shock, obstructive shock and distributive shock, in particular cardiogenic shock, septic shock, shock due to Covid-19, shock due to burns and traumatic shock. A further embodiment of the invention is a pharmaceutical formulation for use in therapy or prevention for use in therapy wherein said patient has shock due chemical contamination with acidic, basic or oxidative reagents and chemicals in solid, liquid or gaseous form.

As used herein, the term “shock” is characterized by decreased oxygen delivery and/or increased oxygen consumption or inadequate oxygen utilization leading to cellular and tissue hypoxia. It is a life-threatening condition of circulatory failure and most commonly manifested as hypotension (systolic blood pressure less than 90 mm Hg or MAP less than 65 mmHg). Shock is divided into four main types based on the underlying cause: hypovolemic, cardiogenic, obstructive, and distributive shock (Vincent and De Backer 2014. N. Engl. J. Med. 370(6): 583).

The term “cardiogenic shock” refers to shock where the patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism. Cardiogenic shock (CS) is defined as a state of critical endorgan hypoperfusion due to reduced cardiac output. Notably, CS forms a spectrum that ranges from mild hypoperfusion to profound shock. Established criteria for the diagnosis of CS are: (i) systolic blood pressure, ≤90 mmHg for >30 min or vasopressors required to achieve a blood pressure ≥90 mmHg; (ii) pulmonary congestion or elevated left-ventricular filling pressures; (iii) signs of impaired organ perfusion with at least one of the following criteria: (a) altered mental status; (b) cold, clammy skin; (c) oliguria (<0.5 mL/kg/h or <30 mL/h); (d) increased serum-lactate (Reynolds and Hochman 2008. Circulation 117: 686-697). Acute myocardial infarction (AMI) with subsequent ventricular dysfunction is the most frequent cause of CS accounting for approximately 80% of cases. Mechanical complications such as ventricular septal (4%) or free wall rupture (2%), and acute severe mitral regurgitation (7%) are less frequent causes of CS after AMI. (Hochman et al. 2000. J Am Coll Cardiol 36: 1063-1070). Non-AMI-related CS may be caused by decompensated valvular heart disease, acute myocarditis, arrhythmias, etc. with heterogeneous treatment options. This translates in 40 000 to 50 000 patients per year in the USA and 60 000 to 70 000 in Europe. Despite advances in treatment mainly by early revascularization with subsequent mortality reduction, CS remains the leading cause of death in AMI with mortality rates still approaching 40-50% according to recent registries and randomized trials (Goldberg et al. 2009. Circulation 119: 1211-1219).

The term “hypovolemic shock” refers to a shock where the patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma. ‘Hypovolemic shock is characterized by decreased intravascular volume and can be divided into two broad subtypes: hemorrhagic and non-hemorrhagic. Common causes of hemorrhagic hypovolemic shock include gastrointestinal bleed, trauma, vascular etiologies (e.g., ruptured abdominal aortic aneurysm, tumor eroding into a major blood vessel) and spontaneous bleeding in the setting of anticoagulant use. Common causes of non-hemorrhagic hypovolemic shock include vomiting, diarrhea, renal loss, skin losses/insensible losses (e.g., burns, heat stroke) or third-space loss in the setting of pancreatitis, cirrhosis, intestinal obstruction, trauma. For review see Koya and Paul 2018. Shock. StatPearls [Internet]. Treasure Island (FL): StatPearls Publishing; 2019-2018 Oct. 27.

The term “obstructive shock” refers to a shock where the patient may have suffered a cardiac tamponade, tension pneumothorax, pulmonary embolism or aortic stenosis. Obstructive shock is due to a physical obstruction of the great vessels or the heart itself. Several conditions can result in this form of shock (e.g., cardiac tamponade, tension pneumothorax, pulmonary embolism, aortic stenosis). For review see Koya and Paul 2018. Shock. StatPearls [Internet]. Treasure Island (FL): StatPearls Publishing; 2019-2018 Oct. 27.

The term “distributive shock” refers to a shock where the patient may have septic shock, neurogenic shock, anaphylactic shock or shock due to adrenal crisis. According to the cause, there are four types of distributive shock: neurogenic shock (decreased sympathetic stimulation leading to decreased vasal tone), anaphylactic shock, septic shock and shock due to adrenal crisis. In addition to sepsis, distributive shock can be caused by systemic inflammatory response syndrome (SIRS) due to conditions other than infection such as pancreatitis, burns or trauma. Other causes include, toxic shock syndrome (TSS), anaphylaxis (a sudden, severe allergic reaction), adrenal insufficiency (acute worsening of chronic adrenal insufficiency, destruction or removal of the adrenal glands, suppression of adrenal gland function due to exogenous steroids, hypopituitarism and metabolic failure of hormone production), reactions to drugs or toxins, heavy metal poisoning, hepatic (liver) insufficiency and damage to the central nervous system. For review see Koya and Paul 2018. Shock. StatPearls [Internet]. Treasure Island (FL): StatPearls Publishing; 2019-2018 Oct. 27.

Refractory shock has been defined as requirement of noradrenaline infusion of >0.5 μg/kg/min despite adequate volume resuscitation. Mortality in these patients may be as high as 94% and the assessment and management of these patients requires a much more aggressive approach for survival. The term “refractory shock” is used when the tissue perfusion cannot be restored with the initial corrective measures employed (e.g., vasopressors) and may therefore be referred to as “high vasopressor-dependent” or “vasopressor-resistant” shock (Udupa and Shetty 2018. Indian J Respir Care 7: 67-72). Patients with refractory shock may have features of inadequate perfusion such as hypotension (mean arterial blood pressure <65 mmHg), tachycardia, cold peripheries, prolonged capillary refill time, and tachypnea consequent to the hypoxia and acidosis. Fever may be seen in septic shock. Other signs of hypoperfusion such as altered sensorium, hyperlactatemia, and oliguria may also be seen. These well-known signs of shock are not helpful in identifying whether the problem is at the pump (heart) or circuitry (vessels and tissues). Different types of shock can coexist, and all forms of shock can become refractory, as evidenced by unresponsiveness to high-dose vasopressors (Udupa and Shetty 2018. Indian J Respir Care 7: 67-72).

Subject matter of the present invention is a pharmaceutical formulation for use in therapy or prevention for use in therapy wherein said patient has shock, wherein said patient is characterized by having a level of dipeptidyl peptidase 3 (DPP3) in a sample of bodily fluid below a threshold prior to drug administration and said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 1).

Subject matter of the present invention is a pharmaceutical formulation for use in therapy or prevention wherein said threshold of DPP3 in a sample of bodily fluid of said patient is between 20 and 120 ng/mL, more preferred between 30 and 80 ng/mL, even more preferred between 40 and 60 ng/mL, most preferred said threshold is 50 ng/mL.

Subject matter of the present invention is a pharmaceutical formulation for use in therapy or prevention wherein the level of DPP3 is determined by contacting said sample of bodily fluid with a capture binder that binds specifically to DPP3.

One embodiment of the present application relates to an anti-ADM antibody or anti-ADM antibody fragment or anti-ADM non-Ig scaffold for use in therapy or prevention of shock in a patient, wherein said shock is selected from the group comprising shock due to hypovolemia, cardiogenic shock, obstructive shock and distributive shock, in particular cardiogenic shock or septic shock.

Another embodiment of the present application relates to an anti-ADM antibody or anti-ADM antibody fragment or anti-ADM non-Ig scaffold for use in therapy or prevention of shock in a patient, wherein
in case of cardiogenic shock said patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

One preferred embodiment of the present application relates to an anti-ADM antibody or anti-ADM antibody fragment or anti-ADM non-Ig scaffold for use in therapy or prevention of shock in a patient, wherein said threshold of DPP3 in a sample of bodily fluid of said patient is between 20 and 120 ng/mL, more preferred between 30 and 80 ng/mL, more preferred between 40 and 90 ng/mL, even more preferred between 40 and 60 ng/mL, most preferred said threshold is 50 ng/mL.

One specific embodiment of the present application relates to an anti-ADM antibody or anti-ADM antibody fragment or anti-ADM non-Ig scaffold for use in therapy or prevention of shock in a patient, wherein the level of DPP3 is determined by contacting said sample of bodily fluid with a capture binder that binds specifically to DPP3.

Another embodiment of the present application relates to an anti-ADM antibody or anti-ADM antibody fragment or anti-ADM non-Ig scaffold for use in therapy or prevention of shock in a patient, wherein either the level of DPP3 protein and/or the level of active DPP3 is determined and compared to a predetermined threshold.

One embodiment of the present application relates to an anti-ADM antibody or anti-ADM antibody fragment or anti-ADM non-Ig scaffold for use in therapy or prevention of shock in a patient, wherein said patient is additionally characterized by having a level of ADM-NH₂ above a threshold.

The level of ADM —NH2 is measured in order to identify patients in shock.

One preferred embodiment of the present application relates to an anti-ADM antibody or anti-ADM antibody fragment or anti-ADM non-Ig scaffold for use in therapy or prevention of shock in a patient, wherein said threshold of ADM-NH₂ in a sample of bodily fluid of said patient is between 40 and 100 μg/mL, more preferred between 50 and 90 μg/mL, even more preferred between 60 and 80 μg/mL, most preferred said threshold is 70 μg/mL.

Another embodiment of the present application relates to an anti-ADM antibody or anti-ADM antibody fragment or anti-ADM non-Ig scaffold for use in therapy or prevention of shock in a patient, wherein the level of ADM-NH₂ is determined by contacting said sample of bodily fluid with a capture binder that binds specifically to ADM-NH₂.

Another preferred embodiment of the present application relates to an anti-ADM antibody or anti-ADM antibody fragment or anti-ADM non-Ig scaffold for use in therapy or prevention of shock in a patient, wherein the sample of bodily fluid of said patient is selected from the group of blood, serum, plasma, urine, cerebrospinal fluid (CSF), and saliva.

Another embodiment of the present application relates to a method of treatment or prevention of shock in a patient, the method comprising administering an anti-adrenomedullin (ADM) antibody or an anti-adrenomedullin antibody fragment or anti-ADM non-Ig scaffold to said patient, the method comprising the steps:
determining the level of DPP3 in a sample of bodily fluid of said subject,
comparing said level of determined DPP3 to a pre-determined threshold, and
wherein said patient is treated if said determined level of DPP3 is below said pre-determined threshold, and
wherein said anti-ADM antibody or anti-ADM fragment or anti-ADM non-Ig scaffold binds to the N-terminal part (aa 1-21) of ADM: YRQSMNNFQGLRSFGCRFGTC (SEQ ID No. 1).

Another specific embodiment of the present application relates to a method of treatment or prevention of shock in a patient, the method comprising administering an anti-adrenomedullin (ADM) antibody or an anti-adrenomedullin antibody fragment or anti-ADM non-Ig scaffold to said patient, wherein the method is additionally comprising the steps of
determining the level of ADM-NH₂ in a sample of bodily fluid of said subject,
comparing said level of ADM-NH₂ to a pre-determined threshold,
wherein said patient is treated if said determined level of ADM-NH₂ is above said pre-determined threshold level.

In one embodiment of the invention either the level of DPP3 protein and/or the level of active DPP3 is determined and compared to a threshold level.

In a specific embodiment of the invention a threshold of DPP3 in a sample of bodily fluid of said patient is between 20 and 120 ng/mL, more preferred between 40 and 90 ng/mL, more preferred between 30 and 80 ng/mL, even more preferred between 40 and 60 ng/mL, most preferred said threshold is 50 ng/mL.

In a specific embodiment of the invention a threshold for the level of DPP3 is the 5fold median concentration, preferably the 4fold median concentration, more preferred the 3fold median concentration, most preferred the 2fold median concentration of a normal healthy population.

The level of DPP3 as the amount of DPP3 protein and/or DPP3 activity in a sample of bodily fluid of said subject may be determined by different methods, e.g., immunoassays, activity assays, mass spectrometric methods etc.

According to the present invention, any types of binding assays (immunoassays and analogous assays, which use other types of antigen-specific binders instead of antibodies), and DPP3 enzyme activity assays, which are specific for DPP3 by specifically capturing DPP3 from a sample using a specific binder (anti-DPP3 antibody or other type of binder) prior to determination of enzyme activity) can be used.

DPP3 activity can be measured by detection of cleavage products of DPP3 specific substrates. Known peptide hormone substrates include Leu-enkephalin, Met-enkephalin, endomorphin 1 and 2, valorphin, β-casomorphin, dynorphin, proctolin, ACTH (Adrenocorticotropic hormone) and MSH (melanocyte-stimulating hormone; Abramić et al. 2000, Baršun et al. 2007, Dhanda et al. 2008). The cleavage of mentioned peptide hormones as well as other untagged oligopeptides (e.g., Ala-Ala-Ala-Ala, Dhanda et al. 2008) can be monitored by detection of the respective cleavage products. Detection methods include, but are not limited to, HPLC analysis (e.g., Lee & Snyder 1982), mass spectrometry (e.g., Abramić et al. 2000), H1-NMR analysis (e.g., Vandenberg et al. 1985), capillary zone electrophoresis (CE; e.g. Baršun et al. 2007), thin layer chromatography (e.g. Dhanda et al. 2008) or reversed phase chromatography (e.g. Mazocco et al. 2006).

Detection of fluorescence due to hydrolysis of fluorogenic substrates by DPP3 is a standard procedure to monitor DPP3 activity. Those substrates are specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) coupled to a fluorophore. Fluorophores include but are not limited to β-naphtylamide (2-naphtylamide, βNA, 2NA), 4-methoxy-β-naphtylamide (4-methoxy-2-naphtylamide) and 7-amido-4-methylcoumarin (AMC, MCA; Abramit et al. 2000, Ohkubo et al. 1999). Cleavage of these fluorogenic substrates leads to the release of fluorescent β-naphtylamine or 7-amino-4-methylcoumarin respectively. In a liquid phase assay or an ECA substrate and DPP3 are incubated in for example a 96 well plate format and fluorescence is measured using a fluorescence detector (Ellis & Nuenke 1967). Additionally, DPP3 carrying samples can be immobilized and divided on a gel by electrophoresis, gels stained with fluorogenic substrate (e.g., Arg-Arg-βNA) and Fast Garnet GBC and fluorescent protein bands detected by a fluorescence reader (Ohkubo et al. 1999). The same peptides (Arg-Arg, Ala-Ala, Ala-Arg, Ala-Phe, Asp-Arg, Gly-Ala, Gly-Arg, Gly-Phe, Leu-Ala,

Leu-Gly, Lys-Ala, Phe-Arg, Suc-Ala-Ala-Phe) can be coupled to chromophores, such as p-nitroanilide diacetate. Detection of color change due to hydrolysis of chromogenic substrates can be used to monitor DPP3 activity.

Another option for the detection of DPP3 activity is a Protease-Glom Assay (commercially available at Promega). In this embodiment of said method DPP3 specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) are coupled to aminoluciferin. Upon cleavage by DPP3, aminoluciferin is released and serves as a substrate for a coupled luciferase reaction that emits detectable luminescence.

In a preferred embodiment DPP3 activity is measured by addition of the fluorogenic substrate Arg-Arg-βNA and monitoring fluorescence in real time.

In a specific embodiment of said method for determining active DPP3 in a bodily fluid sample of a subject said capture binder reactive with DPP3 is immobilized on a solid phase.

The test sample is passed over the immobile binder, and DPP3, if present, binds to the binder and is itself immobilized for detection. A substrate may then be added, and the reaction product may be detected to indicate the presence or amount of DPP3 in the test sample. For the purposes of the present description, the term “solid phase” may be used to include any material or vessel in which or on which the assay may be performed and includes, but is not limited to: porous materials, nonporous materials, test tubes, wells, slides, agarose resins (e.g., Sepharose from GE Healthcare Life Sciences), magnetic particals (e.g. Dynabeadsm or Piercerm magnetic beads from Thermo Fisher Scientific), etc.

In another embodiment of the invention, the level of DPP3 is determined by contacting said sample of bodily fluid with a capture binder that binds specifically to DPP3.

In another preferred embodiment of the invention, said capture binder for determining the level of DPP3 may be selected from the group of antibodies, antibody fragment or non-IgG scaffold.

In a specific embodiment of the invention, said capture binder is an antibody.

The amount of DPP3 protein and/or DPP3 activity in a sample of bodily fluid of said subject may be determined for example by one of the following methods:
1. Luminescence immunoassay for the quantification of DPP3 protein concentrations (LIA) (Rehfeld et al., 2019 JALM 3(6): 943-953).

The LIA is a one-step chemiluminescence sandwich immunoassay that uses white high-binding polystyrene microtiter plates as solid phase. These plates are coated with monoclonal anti-DPP3 antibody AK2555 (capture antibody). The tracer anti-DPP3 antibody AK2553 is labeled with MA70-acridinium-NHS-ester and used at a concentration of 20 ng per well. Twenty microliters of samples (e.g., serum, heparin-plasma, citrate-plasma or EDTA-plasma derived from patients' blood), and calibrators are pipetted into coated white microtiter plates. After adding the tracer antibody AK2553, the microtiter plates are incubated for 3 h at room temperature and 600 rpm. Unbound tracer is then removed by 4 washing steps (350 μL per well). Remaining chemiluminescence is measured for is per well by using a microtiter plate luminometer. The concentration of DPP3 is determined with a 6-point calibration curve. Calibrators and samples are preferably run in duplicate.
2. Enzyme capture activity assay for the quantification of DPP3 activity (ECA) (Rehfeld et al., 2019 JALM 3(6): 943-953).

The ECA is a DPP3-specific activity assay that uses black high-binding polystyrene microtiter plates as solid phase. These plates are coated with monoclonal anti-DPP3 antibody AK2555 (capture antibody). Twenty microliters of samples (e.g., serum, heparin-plasma, citrate-plasma, EDTA-plasma, cerebrospinal fluid and urine) and calibrators are pipetted into coated black microtiter plates. After adding assay buffer (200 μL), the microtiter plates are incubated for 2 h at 22° C. and 600 rpm. DPP3 present in the samples is immobilized by binding to the capture antibody. Unbound sample components are removed by 4 washing steps (350 μL per well). The specific activity of immobilized DPP3 is measured by the addition of the fluorogenic substrate, Arg-Arg-β-Naphthylamide (Arg2-βNA), in reaction buffer followed by incubation at 37° C. for 1 h. DPP3 specifically cleaves Arg2-βNA into Arg-Arg dipeptide and fluorescent β-naphthylamine. Fluorescence is measured with a fluorometer using an excitation wavelength of 340 nm and emission is detected at 410 nm. The activity of DPP3 is determined with a 6-point calibration curve. Calibrators and samples are preferably run in duplicates.
3. Liquid-phase assay for the quantification of DPP3 activity (LAA) (modified from Jones et al., Analytical Biochemistry, 1982).

The LAA is a liquid phase assay that uses black non-binding polystyrene microtiter plates to measure DPP3 activity. Twenty microliters of samples (e.g., serum, heparin-plasma, citrate-plasma) and calibrators are pipetted into non-binding black microtiter plates. After addition of fluorogenic substrate, Arg2-βNA, in assay buffer (200 μL), the initial βNA fluorescence (T=0) is measured in a fluorimeter using an excitation wavelength of 340 nm and emission is detected at 410 nm. The plate is then incubated at 37° C. for 1 hour. The final fluorescence of (T=60) is measured. The difference between final and initial fluorescence is calculated. The activity of DPP3 is determined with a 6-point calibration curve. Calibrators and samples are preferably run in duplicates.

In a specific embodiment an assay is used for determining the level of DPP3, wherein the assay sensitivity of said assay is able to quantify the DPP3 of healthy subjects and is <20 ng/ml, preferably <30 ng/ml and more preferably <40 ng/ml.
4. Another immunoassay method for measuring DPP3 from a plasma of whole blood sample is available, IB10 Sphingotest® DPP3 (https://www.nexus-dx.com/wp-content/uploads/2020/11/DPP3-022-00072-IFU-REV-B_8×11.pdf).

The IB10 Sphingotest® DPP3 is a rapid point-of-care (POC) immunoassay for the in vitro quantitative determination of Dipeptidyl Peptidase 3 (DPP3) in human EDTA whole blood and plasma. The Nexus IB10 immunochemistry system combines chemistry with microfluidics and centrifugal flow to rapidly prepare a cell free plasma from whole blood that can then be moved through a channel to rehydrate, solubilize, and mix with freeze dried immunoconjugates

In a specific embodiment, said binder exhibits a binding affinity to DPP3 of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In another embodiment of the invention, said sample of bodily fluid is selected from the group of whole blood, plasma, and serum.

Mature ADM, bio-ADM and ADM-NH₂ is used synonymously throughout this application and is a molecule according to SEQ ID No.: 13.

A bodily fluid according to the present invention is in one particular embodiment a blood sample. A blood sample may be selected from the group comprising whole blood, serum and plasma. In a specific embodiment of the method said sample is selected from the group comprising human citrate plasma, heparin plasma and EDTA plasma.

In a specific embodiment an assay is used for determining the level ADM-NH₂, wherein the assay sensitivity of said assay is able to quantify the mature ADM-NH₂ of healthy subjects and is <70 μg/ml, preferably <40 μg/ml and more preferably <10 μg/ml.

In a specific embodiment of the invention the threshold for ADM-NH₂ is between 40 and 100 μg/mL, more preferred between 50 and 90 μg/mL, even more preferred between 60 and 80, most preferred a threshold of 70 μg/ml is applied.

In a specific embodiment of the invention a threshold for plasma ADM-NH₂ is the 5fold median concentration, preferably the 4fold median concentration, more preferred the 3fold median concentration, most preferred the 2fold median concentration of a normal healthy population.

In a specific embodiment, said binder exhibits a binding affinity to ADM-NH₂ of at least 10⁷ M⁻¹, preferred 10⁸ M⁻¹, preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

As used herein, the term “binding affinity” refers to the affinity of the proteins described in the present invention to their binding targets and is expressed numerically using “Kd” values. The term “Kd”, as used herein, is intended to refer to the dissociation constant of an antibody-antigen interaction. If two or more proteins are indicated to have comparable binding affinities towards their binding targets, then the Kd values for binding of the respective proteins towards their binding targets, are within ±2-fold of each other. If two or more proteins are indicated to have comparable binding affinities towards single binding target, then the Kd values for binding of the respective proteins towards said single binding target, are within ±2-fold of each other. If a protein is indicated to bind two or more targets with comparable binding affinities, then the Kd values for binding of said protein to the two or more targets are within +2-fold of each other. In general, a higher Kd value corresponds to a weaker binding. In some embodiments, the “Kd” is measured by a radiolabeled antigen binding assay (MA) or surface plasmon resonance assays using a BIAcore™-2000 or a BIAcore™-3000 (BIAcore, Inc., Piscataway, N.J.).

In a specific embodiment, said binder is selected from the group comprising an antibody or an antibody fragment or a non-Ig scaffold binding to ADM-NH₂.

In a specific embodiment an assay is used for determining the level of ADM-NH₂, wherein such assay is a sandwich assay, preferably a fully automated assay.

In one embodiment such assay for determining the level of the biomarkers (DPP3 and/or ADM-NH₂) is a sandwich immunoassay using any kind of detection technology including but not restricted to enzyme label, chemiluminescence label, electrochemiluminescence label, preferably a fully automated assay. In one embodiment of the diagnostic method such an assay is an enzyme labeled sandwich assay. Examples of automated or fully automated assay comprise assays that may be used for one of the following systems: Roche Elecsys®, Abbott Architect@, Siemens Centauer®, Brahms Kryptor®, BiomerieuxVidas®, Alere Triage®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromatography assays.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

In one embodiment of the invention, it may be a so-called POC-test (point-of-care) that is a test technology, which allows performing the test within less than 1 hour near the patient without the requirement of a fully automated assay system. One example for this technology is the immunochromatographic test technology.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type. In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I Kroschwitz; editor, M. Howe-Grant, John Wiley & Sons, 1993, vol. 15, p. 518-562, incorporated herein by reference, including citations on pages 551-562).

Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In a specific embodiment at least one of said two binders is labeled in order to be detected.

The ADM-NH₂ levels of the present invention have been determined with the described ADM-NH₂ assay (Weber et al. 2017. JALM 2(2):1-4). The DPP3 levels of the present invention have been determined with the described DPP3-assays as outlined in the examples (Rehfeld et al. 2019. JALM 3(6): 943-953). The mentioned threshold values above might be different in other assays, if these have been calibrated differently from the assay systems used in the present invention. Therefore, the mentioned cut-off values above shall apply for such differently calibrated assays, accordingly, taking into account the differences in calibration. One possibility of quantifying the difference in calibration is a method comparison analysis (correlation) of the assay in question with the respective biomarker assay used in the present invention by measuring the respective biomarker (e.g., bio-ADM, DPP3) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from normal (healthy) subjects have been measured: median plasma bio-ADM (mature ADM-NH₂) was 24.7 μg/ml, the lowest value 11 μg/ml and the 99^{th} percentile 43 μg/ml (Marino et al. 2014. Critical Care 18:R34). With the calibration used in the present invention, samples from 5,400 normal (healthy) subjects (Swedish single-center prospective population-based Study (MPP-RES)) have been measured: median (interquartile range) plasma DPP3 was 14.5 ng/ml (11.3 ng/ml-19 ng/ml).

In the present invention, the level of ADM —NH2 is therefore measured in order to identify patients having an increased risk of running into shock.

The term “prevention” or any grammatical variation thereof (e.g., prevent, preventing, and prevention etc.), as used herein, includes but is not limited to, delaying the onset of symptoms, preventing relapse to a disease, increasing latency between symptomatic episodes, or a combination thereof. Prevention, as used herein, does not require the complete absence of symptoms.

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Ex, 1(1):22; Wagner et al. 2013. Intensive Care Med Exp 1(1):21).

Due to these positive results, a humanized version of an N-terminal anti-ADM antibody, named Adrecizumab, has been developed for further clinical development. Beneficial effects of Adrecizumab on vascular barrier function and survival were recently demonstrated in preclinical models of systemic inflammation and sepsis (Geven et al. 2018. Shock 50(6):648-654). In this study, pre-treatment with Adrecizumab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Adrecizumab improved 7-day survival in CLP-induced sepsis in mice from 10 to 50% for single and from 0 to 40% for repeated dose administration. Moreover, in a phase I study, excellent safety and tolerability was demonstrated (see Example 6): no serious adverse events were observed, no signal of adverse events occurring more frequently in Adrecizumab-treated subjects was detected and no relevant changes in other safety parameters were found (Geven et al. 2017. Intensive Care Med Exp 5 (Suppl 2): 0427). Of particular interest is the proposed mechanism of action of Adrecizumab. Both animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2):132-140 and Voors et al (J. Eur J Heart Fail. 2019 February; 21(2):163-171)). In addition, binding of the antibody to ADM leads to a prolongation of ADM's half-life. Even though NT-ADM antibodies partially inhibit ADM-mediated signalling, a significant increase of circulating ADM results in an overall “net” increase of ADM activity in the blood compartment, where it exerts beneficial effects on ECs (predominantly barrier stabilization), whereas ADMs detrimental effects on VSMCs (vasodilation) in the interstitium are reduced.

The invention is not limited to the use of Adrecizumab specifically. There is no reason to doubt that what is true for Adrecizumab will also be true for antibodies sharing main essential features (in particular affinity and epitope specificity) Antibodies that target the same region must be expected to have the same technical effect, provided they have the same affinity and same or very comparable structural features (size, shape, . . . ).

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-Adrenomedullin (ADM) antibody or anti-adrenomedullin antibody fragment or anti-ADM non-Ig scaffold is monospecific.

Monospecific anti-adrenomedullin (ADM) antibody or monospecific anti-adrenomedullin antibody fragment or monospecific anti-ADM non-Ig scaffold means that said antibody or antibody fragment or non-Ig scaffold binds to one specific region encompassing at least 5 amino acids within the target ADM. Monospecific anti-Adrenomedullin (ADM) antibody or monospecific anti-adrenomedullin antibody fragment or monospecific anti-ADM non-Ig scaffold are anti-adrenomedullin (ADM) antibodies or anti-adrenomedullin antibody fragments or anti-ADM non-Ig scaffolds that all have affinity for the same antigen. Monoclonal antibodies are monospecific, but monospecific antibodies may also be produced by other means than producing them from a common germ cell.

Said anti-ADM antibody or antibody fragment binding to ADM or non-Ig scaffold binding to ADM may be a non-neutralizing anti-ADM antibody or antibody fragment binding to ADM or non-Ig scaffold binding to ADM.

In a specific embodiment said anti-ADM antibody, anti-ADM antibody fragment or anti-ADM non-Ig scaffold is a non-neutralizing antibody, fragment or non-Ig scaffold. A neutralizing anti-ADM antibody, anti-ADM antibody fragment or anti-ADM non-Ig scaffold would block the bioactivity of ADM to nearly 100%, to at least more than 90%, preferably to at least more than 95%.

In contrast, a non-neutralizing anti-ADM antibody, or anti-ADM antibody fragment or anti-ADM non-Ig scaffold blocks the bioactivity of ADM less than 100%, preferably to less than 95%, preferably to less than 90%, more preferred to less than 80% and even more preferred to less than 50%. This means that bioactivity of ADM is reduced to less than 100%, to 95% or less but not more, to 90% or less but not more, to 80% or less but not more, to 50% or less but not more This means that the residual bioactivity of ADM bound to the non-neutralizing anti-ADM antibody, or anti-ADM antibody fragment or anti-ADM non-Ig scaffold would be more than 0%, preferably more than 5%, preferably more than 10%, more preferred more than 20%, more preferred more than 50%.

In this context (a) molecule(s), being it an antibody, or an antibody fragment or a non-Ig scaffold with “non-neutralizing anti-ADM activity”, collectively termed here for simplicity as “non-neutralizing” anti-ADM antibody, antibody fragment, or non-Ig scaffold, that e.g., blocks the bioactivity of ADM to less than 80%, is defined as
a molecule or molecules binding to ADM, which upon addition to a culture of an eukaryotic cell line, which expresses functional human recombinant ADM receptor composed of CRLR (calcitonin receptor like receptor) and RAMP3 (receptor-activity modifying protein 3), reduces the amount of cAMP produced by the cell line through the action of parallel added human synthetic ADM peptide, wherein said added human synthetic ADM is added in an amount that in the absence of the non-neutralizing antibody to be analyzed, leads to half-maximal stimulation of cAMP synthesis, wherein the reduction of cAMP by said molecule(s) binding to ADM takes place to an extent, which is not more than 80%, even when the non-neutralizing molecule(s) binding to ADM to be analyzed is added in an amount, which is 10-fold more than the amount, which is needed to obtain the maximal reduction of cAMP synthesis obtainable with the non-neutralizing antibody to be analyzed.

The same definition applies to the other ranges; 95%, 90%, 50% etc.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG3, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acids in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988. Proc. Natl. Acad. Sci. U.S.A., 85:5879-5883; Bird et al. 1988. Science 242:423-426; Hood et al. 1984, Immunology, Benjamin, N.Y., 2nd ed.; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al. 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., WO91/17271; WO92/001047; WO92/20791) or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-ADM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins and numerous others.

In addition to anti-ADM antibodies other biopolymer scaffolds are well known in the art to complex a target molecule and have been used for the generation of highly target specific biopolymers. Examples are aptamers, spiegelmers, anticalins and conotoxins. For illustration of antibody formats please see FIGS. 1a, 1b and 1c.

In a preferred embodiment the anti-ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1 266 025; lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867).

In one embodiment of the invention anti-ADM antibodies according to the present invention may be produced as outlined in Example 1 by synthesizing fragments of ADM as antigens. Thereafter, binder to said fragments are identified using the below described methods or other methods as known in the art.

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Humanization of antibodies. Front Biosci. 2008 Jan. 1; 13:1619-33).

In a preferred embodiment the ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format.

In another preferred embodiment, the anti-ADM antibody, anti-ADM antibody fragment, or anti-ADM non-Ig scaffold is a full-length antibody, antibody fragment, or non-Ig scaffold.

In a preferred embodiment the anti-ADM antibody or an anti-ADM antibody fragment or anti-ADM non-Ig scaffold is directed to and can bind to an epitope of at least 5 amino acids in length contained in ADM.

In a more preferred embodiment, the anti-ADM antibody or an anti-ADM antibody fragment or anti-ADM non-Ig scaffold is directed to and can bind to an epitope of at least 4 amino acids in length contained in ADM.

In one specific embodiment of the invention the anti-ADM antibody or anti-ADM antibody fragment binding to adrenomedullin or anti-ADM non-Ig scaffold binding to adrenomedullin is provided for use in therapy or prevention of shock in a patient, wherein said antibody or fragment or scaffold is not ADM-binding-Protein-1 (complement factor H).

In one specific embodiment of the invention the anti-Adrenomedullin (ADM) antibody or anti-ADM antibody fragment binding to adrenomedullin or anti-ADM non-Ig scaffold binding to adrenomedullin is provided for use in therapy or prevention of shock in a patient, wherein said antibody or fragment or scaffold binds to a region of preferably at least 4, or at least 5 amino acids within the sequence of amino acid 1-21 of mature human ADM: YRQSMNNFQGLRSFGCRFGTC SEQ ID No.: 1

In a preferred embodiment of the present invention said anti-ADM antibody or anti-adrenomedullin antibody fragment or anti-ADM non-Ig scaffold binds to a region or epitope of ADM that is located in the N-terminal part (amino acid 1-21) of adrenomedullin.

In another preferred embodiment said anti-ADM-antibody or anti-ADM antibody fragment or anti-ADM non-Ig scaffold recognizes and binds to a region or epitope within amino acids 1-14 of adrenomedullin: YRQSMNNFQGLRSF (SEQ ID No.: 15) that means to the N-terminal part (amino acid 1-14) of adrenomedullin.

In another preferred embodiment said anti-ADM-antibody or anti-adrenomedullin antibody fragment or anti-ADM non-Ig scaffold recognizes and binds to a region or epitope within amino acids 1-10 of adrenomedullin: YRQSMNNFQG (SEQ ID No.: 16); that means to the N-terminal part (amino acid 1-10) of adrenomedullin.

In another preferred embodiment said anti-ADM antibody or anti-ADM antibody fragment or anti-ADM non-Ig scaffold recognizes and binds to a region or epitope within amino acids 1-6 of adrenomedullin: YRQSMN (SEQ ID No.: 17); that means to the N-terminal part (amino acid 1-6) of adrenomedullin. As stated above said region or epitope comprises preferably at least 4 or at least 5 amino acids in length.

In another preferred embodiment said anti-ADM antibody or anti-ADM antibody fragment or anti-ADM non-Ig scaffold recognizes and binds to the N-terminal end (amino acid 1) of adrenomedullin. N-terminal end means that the amino acid 1, that is “Y” of SEQ ID No. 13, or 18 (SEQ ID No.: 18 (1-42 of human ADM): YRQSMNNFQGLRSFGCRFGTCTVQKLAHQIYQFTDKDKDNVA), respectively and is mandatory for binding. The antibody or fragment or scaffold would neither bind N-terminal extended nor N-terminal modified Adrenomedullin nor N-terminal degraded adrenomedullin. This means in another preferred embodiment said anti-ADM-antibody or anti-ADM antibody fragment or anti-ADM non-Ig scaffold binds only to a region within the sequence of mature ADM if the N-terminal end of ADM is free. In said embodiment the anti-ADM antibody or anti-ADM antibody fragment or non-Ig scaffold would not bind to a region within the sequence of mature ADM if said sequence is e.g., comprised within pro-ADM.

For the sake of clarity, the numbers in brackets for specific regions of ADM like “N-terminal part (amino acid 1-21)” is understood by a person skilled in the art that the N-terminal part of ADM consists of amino acids 1-21 of the mature ADM sequence.

In another specific embodiment pursuant to the invention the herein provided anti-ADM antibody or anti-ADM antibody fragment or anti-ADM non-Ig scaffold does not bind to the C-terminal portion of ADM, i.e., the amino acid 43-52 of ADM:

(SEQ ID No.: 19)
PRSKISPQGY-NH₂.

An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes, and linear epitopes, based on their structure and interaction with the paratope.

Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontinuous amino acid residues. A linear or a sequential epitope is an epitope that is recognized by antibodies by its linear sequence of amino acids, or primary structure and is formed by the 3-D conformation adopted by the interaction of contiguous amino acid residues.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold leads to an increase of the ADM level or ADM immunoreactivity in serum, blood, plasma of at least 10%, preferably at least 50%, more preferably >50%, most preferably >100%.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold is an ADM stabilizing antibody or an ADM stabilizing antibody fragment or an ADM stabilizing non-Ig scaffold that enhances the half-life (t_{1/2}; half retention time) of adrenomedullin in serum, blood, plasma at least 10%, preferably at least 50%, more preferably >50%, most preferably >100%.

The half-life (half retention time) of ADM may be determined in human serum, blood or plasma in absence and presence of an ADM stabilizing antibody or an ADM stabilizing antibody fragment or an ADM stabilizing non-Ig scaffold, respectively, using an immunoassay for the quantification of ADM. The following steps may be conducted:
ADM may be diluted in human citrate plasma in absence and presence of an ADM stabilizing antibody or an adrenomedullin stabilizing antibody fragment or an adrenomedullin stabilizing non-Ig scaffold, respectively, and may be incubated at 24° C.
Aliquots are taken at selected time points (e.g., within 24 hours) and degradation of ADM may be stopped in said aliquots by freezing at −20° C.
The quantity of ADM may be determined by a hADM immunoassay directly if the selected assay is not influenced by the stabilizing antibody. Alternatively, the aliquot may be treated with denaturing agents (like HCl) and, after clearing the sample (e.g., by centrifugation) the pH can be neutralized and the ADM-quantified by an ADM immunoassay. Alternatively, non-immunoassay technologies (e.g., RP-HPLC) can be used for ADM-quantification.
The half-life of ADM is calculated for ADM incubated in absence and presence of an ADM stabilizing antibody or an adrenomedullin stabilizing antibody fragment or an adrenomedullin stabilizing non-Ig scaffold, respectively.
The enhancement of half-life is calculated for the stabilized ADM in comparison to ADM that has been incubated in absence of an ADM stabilizing antibody or an adrenomedullin stabilizing antibody fragment or an adrenomedullin stabilizing non-Ig scaffold.

A two-fold increase of the half-life of ADM is an enhancement of half-life of 100%.

Half-life (half retention time) is defined as the period over which the concentration of a specified chemical or drug takes to fall to half its baseline concentration in the specified fluid or blood.

An assay that may be used for the determination of the half-life (half retention time) of adrenomedullin in serum, blood, plasma is described in Example 3.

In a preferred embodiment said anti-ADM antibody, anti-ADM antibody fragment or anti-ADM non-Ig scaffold is a non-neutralizing antibody, fragment, or scaffold. A neutralizing anti-ADM antibody, anti-ADM antibody fragment or anti-ADM non-Ig scaffold would block the bioactivity of ADM to nearly 100%, to at least more than 90%, preferably to at least more than 95%. In other words, this means that said non-neutralizing anti-ADM antibody, anti-ADM antibody fragment or anti-ADM non-Ig scaffold blocks the bioactivity of ADM to less than 100%, preferably less than 95% preferably less than 90%. In an embodiment wherein said non-neutralizing anti-ADM antibody, anti-ADM antibody fragment or anti-ADM non-Ig scaffold blocks the bioactivity of ADM to less than 95% an anti-ADM antibody, anti-ADM antibody fragment or anti-ADM non-Ig scaffold that would block the bioactivity of ADM to more than 95% would be outside of the scope of said embodiment. This means in one embodiment that the bioactivity is reduced to 95% or less but not more, preferably to 90% or less, more preferably to 80% or less, more preferably to 50% or less but not more.

In one embodiment of the invention the non-neutralizing antibody is an antibody binding to a region of at least 5 amino acids within the sequence of amino acid 1-21 of mature human ADM (SEQ ID No.: 1).

In another preferred embodiment of the invention the non-neutralizing antibody is an antibody binding to a region of at least 4 amino acids within the sequence of amino acid 1-21 of mature human ADM (SEQ ID No.: 1).

In a specific embodiment according to the present invention a non-neutralizing anti-ADM antibody or anti-ADM antibody fragment or ADM non-Ig scaffold is used, wherein said anti-ADM antibody or an anti-ADM antibody fragment blocks the bioactivity of ADM to less than 80%, preferably less than 50% (of baseline values). It has to be understood that said limited blocking of the bioactivity (meaning reduction of the bioactivity) of ADM occurs even at excess concentration of the antibody, fragment or scaffold, meaning an excess of the antibody, fragment or scaffold in relation to ADM. Said limited blocking is an intrinsic property of the ADM binder itself in said specific embodiment. This means that said antibody, fragment or scaffold has a maximal inhibition of 80% or 50% respectively. In a preferred embodiment said anti-ADM antibody, anti-ADM antibody fragment or anti-ADM non-Ig scaffold would block the bioactivity/reduce the bioactivity of anti-ADM to at least 5%. The stated above means that approximately 20% or 50% or even 95% residual ADM bioactivity remains present, respectively.

Thus, in accordance with the present invention the provided anti-ADM antibodies, anti-ADM antibody fragments, and anti-ADM non-Ig scaffolds do not neutralize the respective ADM bioactivity.

The bioactivity is defined as the effect that a substance takes on a living organism or tissue or organ or functional unit in vivo or in vitro (e.g., in an assay) after its interaction. In case of ADM bioactivity this may be the effect of ADM in a human recombinant ADM receptor cAMP functional assay. Thus, according to the present invention bioactivity is defined via an ADM receptor cAMP functional assay. The following steps may be performed in order to determine the bioactivity of ADM in such an assay:
Dose response curves are performed with ADM in said human recombinant ADM receptor cAMP functional assay.
The ADM concentration of half-maximal cAMP stimulation may be calculated.
At constant half-maximal cAMP-stimulating ADM concentrations dose response curves (up to 100 μg/ml final concentration) are performed by an ADM stabilizing antibody or ADM stabilizing antibody fragment or ADM stabilizing non-Ig scaffold, respectively.

A maximal inhibition in said ADM bioassay of 50% means that said anti-ADM antibody or said anti-ADM antibody fragment or said anti-ADM non-Ig scaffold, respectively, blocks the bioactivity of ADM to 50% of baseline values. A maximal inhibition in said ADM bioassay of 80% means that said anti-ADM antibody or said anti-adrenomedullin antibody fragment or said anti-adrenomedullin non-Ig scaffold, respectively, blocks the bioactivity of ADM to 80%. This is in the sense of blocking the ADM bioactivity to not more than 80%. This means approximately 20% residual ADM bioactivity remains present.

However, by the present specification and in the above context the expression “blocks the bioactivity of ADM” in relation to the herein disclosed anti-ADM antibodies, anti-ADM antibody fragments, and anti-ADM non-Ig scaffolds should be understood as mere decreasing the bioactivity of ADM from 100% to 20% remaining ADM bioactivity at maximum, preferably decreasing the ADM bioactivity from 100% to 50% remaining ADM bioactivity; but in any case there is ADM bioactivity remaining that can be determined as detailed above.

The bioactivity of ADM may be determined in a human recombinant Adrenomedullin receptor cAMP functional assay (Adrenomedullin Bioassay) according to Example 2.

In a preferred embodiment a modulating anti-ADM antibody or a modulating anti-ADM antibody fragment or a modulating anti-ADM non-Ig scaffold is used in therapy or prevention of shock in a patient.

A “modulating” anti-ADM antibody or a modulating anti-ADM antibody fragment or a modulating anti-ADM non-Ig scaffold is an antibody or antibody fragment or non-Ig scaffold that enhances the half-life (t_{1/2} half retention time) of adrenomedullin in serum, blood, plasma at least 10%, preferably at least, 50%, more preferably >50%, most preferably >100% and blocks the bioactivity of ADM to less than 80%, preferably less than 50% and said anti-ADM antibody, anti-ADM antibody fragment or anti-ADM non-Ig scaffold would block the bioactivity of ADM to at least 5%. These values related to half-life and blocking of bioactivity have to be understood in relation to the before-mentioned assays in order to determine these values. This is in the sense of blocking the ADM bioactivity of not more than 80% or not more than 50%, respectively.

Such a modulating anti-ADM antibody or modulating anti-ADM antibody fragment or a modulating anti-ADM non-Ig scaffold offers the advantage that the dosing of the administration is facilitated. The combination of partially blocking or partially reducing ADM bioactivity and increase of the in vivo half-life (increasing the ADM bioactivity) leads to beneficial simplicity of anti-ADM antibody or an anti-ADM antibody fragment or anti-ADM non-Ig scaffold dosing. In a situation of excess of endogenous ADM (maximal stimulation, late sepsis phase, shock, hypodynamic phase) the activity lowering effect is the major impact of the antibody or fragment or scaffold, limiting the (negative) effect of ADM. In case of low or normal endogenous ADM concentrations, the biological effect of anti-ADM antibody or anti-ADM antibody fragment or anti-ADM non-Ig scaffold is a combination of lowering (by partially blocking) and increase by increasing the ADM half-life. Thus, the non-neutralizing and modulating anti-ADM antibody or anti-ADM antibody fragment or anti-ADM non-Ig scaffold acts like an ADM bioactivity buffer in order to keep the bioactivity of ADM within a certain physiological range.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The present invention also relates to a pharmaceutical formulation comprising a therapeutically effective dose of the active ingredient, in combination with at least one pharmaceutically acceptable excipient.

“Pharmaceutically acceptable excipient” refers to an excipient that does not produce an adverse, allergic, or other untoward reaction when administered to a subject. It includes in addition to a therapeutic protein, carriers, various diluents, fillers, salts, buffers, stabilizers, solubilizers, and other materials well known in the art. The characteristics of the carrier will depend on the route of administration.

In some embodiments, the formulation of the present invention can be administered directly into the blood stream, into muscle, into tissue, into fat or into an internal organ of a patient. Suitable means for parenteral administration include intravenous, intraarterial, intraperitoneal, intrathecal, intraventricular, intraurethral, intrasternal, intracranial, intramuscular, intra-ossial, intradermal and subcutaneous. Suitable devices for parenteral administration include needle (including microneedle, micro projections, soluble needles and other micropore formation techniques) injectors, needle-free injectors, and infusion techniques. In some embodiments, the formulation of the present invention is administered to the subject subcutaneously.

In a further embodiment, the aqueous pharmaceutical composition can be administered to a subject in need of treatment, in accordance with known methods of administration. Examples of methods of administration can further include shock dose injection or infusion over a certain period of time, intracerebrospinal, transdermal, oral, topical application or inhalation.

In some embodiments, the administration pattern of the aqueous formulation according to the present invention comprises administration of a dose of the formulation once every week, once every two weeks, once every three weeks, once every four weeks, once every five weeks, once every six weeks, once every seven weeks, once every eight weeks, once every nine weeks, once every ten weeks, once every fifteen weeks, once every twenty weeks or once every twenty-four weeks. In some embodiments, the aqueous formulation according to the present invention is administered once every month, once every two months, once every three months, once every four months, once every five months, or once every six months.

In some embodiments the volume of a dose in the formulation is less than or equal to about 20 ml, about 15 ml, about 10 ml, about 5 ml, about 2.5 ml, about 1 0.5 ml, about 1 0.0 ml, about 0.75 ml, about 0.5 ml, about 0.25 ml or about 0.01 ml.

In some embodiments, the volume of a dose in the formulation is about 20 ml, about 19 ml, about 18 ml, about 17 ml, about 16 ml, about 15 ml, about 14 ml, about 13 ml, about 12 ml, about 1 1 ml, about 10 ml, about 9 ml, about 8 ml, about 7 ml, about 6 ml, about 5 ml, about 4 ml, about 3 ml, about 2 ml or about 1 ml. Alternatively about 20.5 ml, about 19.5 ml, about 18.5 ml, about 17.5 ml, about 16.5 ml, about 15.5 ml, about 14.5 ml, about 13.5 ml, about 12.5 ml, about 1 1 0.5 ml, about 10.5 ml, about 9.5 ml, about 8.5 ml, about 7.5 ml, about 6.5 ml, about 5.5 ml, about 4.5 ml, about 3.5 ml, about 2.5 ml, about 1 0.5 ml, or about 0.5. Alternatively, about 900 microliters, about 800 microliters, about 700 microliters, about 600 microliters, about 500 microliters, about 400 microliters, about 300 microliters, about 200 microliters, or about 100 microliters, alternatively about 950 microliters, about 850 microliters, about 750 microliters, about 650 microliters, about 550 microliters, about 450 microliters, about 350 microliters, about 250 microliters, about 150 microliters, or about 50 microliters. In some embodiments, the volume of the dose in a formulation is about 1 0.0 ml.

Dosage regimens may depend on the pattern of pharmacokinetic decay that the practitioner wishes to achieve. For example, in some embodiments, dosing from one-four times a week is contemplated. Even less frequent dosing may be used. In some embodiments, the dose is administered once every 1 week, every 2 weeks, every 3 weeks, every 4 weeks, every 5 weeks, every 6 weeks, every 7 weeks, every 8 weeks, every 9 weeks, every 10 weeks, every 15 weeks, every 20 weeks, every 25 weeks, or longer. In some embodiments, the dose is administered once every 1 month, every 2 months, every 3 months, every 4 months, every 5 months, every 6 months, or longer. The progress of this therapy is easily monitored by conventional techniques and assays. The dosing regimen can vary over time. For example, in some embodiments, the dose of the formulation of the present invention is administered at 1-10 mg/kg body weight every other week (e.g., by intravenous injection). In some embodiments, the dose of the formulation of the present invention is administered 1-10 mg/kg body weight every other week (e.g., by intravenous injection). In some embodiments, the dose of the formulation of the present invention is administered at an initial dose of four 1-10 mg/kg body weight intravenous injections in one day (at day 1) or two 1-10 mg/kg body weight injections per day for two consecutive days (at days 1 and 2), followed by a second dose two weeks later (at day 15) at 1-10 mg/kg body weight, and followed by a maintenance dose of 1-10 mg/kg body weight every other week. In some embodiments, the dose of the formulation of the present invention is administered at an initial dose of 1-10 mg/kg body weight (e.g., intravenous injection), followed by 1-10 mg/kg body weight every other week starting one week after initial dose. For the purpose of the present invention, the appropriate dosage of the medicament will depend on the antibody employed, the type and severity of the disorder to be treated, whether the agent is administered for preventative or therapeutic purposes, previous therapy, the patient's clinical history and response to the agent, and the discretion of the attending physician. Typically, the clinician will administer the medicament, until a dosage is reached that achieves the desired result. Dosages may be determined empirically. For example, individuals are given incremental dosages to assess efficacy of the medicament, blood glucose levels may be followed.

Dose and/or frequency can vary over course of treatment. Empirical considerations, such as the antibody half-life, generally will contribute to the determination of the dosage. Frequency of administration may be determined and adjusted over the course of therapy, and is generally, but not necessarily, based on treatment and/or suppression and/or amelioration and/or delay of one or more symptoms of autoimmune disease. In some individuals, more than one dose may be required. Frequency of administration may be determined and adjusted over the course of therapy. For example, without limitation, for repeated administrations over several days or longer, depending on the disease and its severity, the treatment is sustained until a desired suppression of symptoms occurs or until sufficient therapeutic levels are achieved.

Administration of the formulation of the present invention can be continuous or intermittent, depending, for example, upon the recipient's physiological condition, whether the purpose of the administration is therapeutic or prophylactic, and other factors known to skilled practitioners. The administration of the formulation of the present invention can be essentially continuous over a preselected period of time or may be in a series of spaced dose.

Preferably the administration of the dose is a parenteral administration preferably selected from intravenous, intraarterial, intraperitoneal, intrathecal, intraventricular, intraurethral, intrasternal, intracranial, intramuscular, intra-ossial, intradermal and subcutaneous. Preferably the formulation is in a unit dosage sterile form for parenteral administration (e.g., subcutaneous administration).

The following embodiments are also subject of the present invention:
1. Pharmaceutical aqueous formulation comprising an human or humanized anti-adrenomedullin (ADM) antibody or an anti-adrenomedullin antibody fragment wherein said antibody or fragment binds to the N-terminal part (aa 1-21) of ADM: YRQSMNNFQGLRSFGCRFGTC (SEQ ID No. 1), wherein said antibody or antibody fragment is present in a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml and wherein said pharmaceutical aqueous formulation is further comprising:
   Arginine in a range of 1 g/L to 100 g/L, and
   Trehalose in a range of 1 g/L to 100 g/L, and
   A surfactant selected from polysorbates and poloxamers in a range of 0.01 g/L to 5 g/L, and
   Histidine in a range of 0.1 g/L to 6.4 g/L, and wherein the pH of the formulation is in the range of 4.0 to 8.0.
2. Pharmaceutical aqueous formulation comprising a human or humanized anti-adrenomedullin (ADM) antibody or an anti-adrenomedullin antibody fragment according to embodiment 1, wherein said antibody or fragment is a human monoclonal antibody or fragment that binds to the N-terminal region (aa 1-21) of ADM (SEQ ID No. 1) or an antibody fragment thereof wherein the heavy chain comprises the sequences:

CDR1:
SEQ ID NO: 2
GYTFSRYW
CDR2:
SEQ ID NO: 3
ILPGSGST
CDR3:
SEQ ID NO: 4
TEGYEYDGFDY

and wherein the light chain comprises the sequences:

CDR1:
SEQ ID NO: 5
QSIVYSNGNTY
CDR2:
SEQ ID NO: 6
RVS
CDR3:
SEQ ID NO: 7
FQGSHIPYT.

3. Pharmaceutical aqueous formulation comprising a human or humanized anti-adrenomedullin (ADM) antibody or an anti-adrenomedullin antibody fragment according to any of embodiments 1 to 2, comprising an Anti-Adrenomedullin antibody directed to the N-terminal end of Adrenomedullin comprising the following sequence as a heavy chain:

SEQ ID NO: 8
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

or a sequence that is >95% identical to it,
and comprises the following sequence as a light chain:

SEQ ID NO: 9
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >95% identical to it.
4. Pharmaceutical aqueous formulation comprising a human or humanized anti-adrenomedullin (ADM) antibody or an anti-adrenomedullin antibody fragment according to any of embodiments 1 to 3, wherein said surfactant is Poloxamer.
5. Pharmaceutical aqueous formulation comprising a human or humanized anti-adrenomedullin (ADM) antibody or an anti-adrenomedullin antibody fragment according to any of embodiments 1 to 4, wherein said formulation is essentially free of NaCl and/or Glycine.
6. Pharmaceutical aqueous formulation comprising a human or humanized anti-adrenomedullin (ADM) antibody or an anti-adrenomedullin antibody fragment according to any of embodiments 1 to 5, wherein Arginine is present in a range of 1 g/L to 100 g/L, preferably 15 g/L to 60 g/L, preferably 13.1 g/L to 52.2 g/L, preferably 11.2 g/L to 44.4 g/L and more preferably 26.1 g/L.
7. Pharmaceutical aqueous formulation comprising a human or humanized anti-adrenomedullin (ADM) antibody or an anti-adrenomedullin antibody fragment according to any of embodiments 1 to 6, wherein Trehalose is present in a range of 1 g/L to 100 g/L, preferably 32.6 g/L to 97.8 g/L, preferably 28.4 g/L to 85.1 g/L, preferably 24.1 g/L to 72.4 g/L and more preferably 56.7 g/L.
8. Pharmaceutical aqueous formulation comprising a human or humanized anti-adrenomedullin (ADM) antibody or an anti-adrenomedullin antibody fragment according to any of embodiments 1 to 7, wherein Poloxamer is present in a range of 0.01 g/L to 5 g/L, preferably 0.115 g/L to 1.15 g/L, preferably 0.1 g/L to 1.0 g/L, preferably 0.085 g/L to 0.85 g/L and more preferably 0.5 g/L.
9. Pharmaceutical aqueous formulation comprising a human or humanized anti-adrenomedullin (ADM) antibody or an anti-adrenomedullin antibody fragment according to any of embodiments 1 to 8, wherein Histidine is present in a range of 0.1 g/L to 6.4 g/L, preferably 0.22 g/L to 6.88 g/L, preferably 0.8 g/L to 3.2 g/L, preferably 0.68 g/L to 3.68 g/L and more preferably 1.6 g/L.
10. Pharmaceutical aqueous formulation comprising a human or humanized anti-adrenomedullin (ADM) antibody or an anti-adrenomedullin antibody fragment according to any of embodiments 1 to 9, wherein the pH of the formulation is in the range of 4.0 to 8.0 preferably 5.0 to 7.0, more preferably 6.0.
11. Pharmaceutical aqueous formulation comprising an human or humanized anti-adrenomedullin (ADM) antibody or an anti-adrenomedullin antibody fragment according to any of embodiments 1 to 10, wherein said formulation comprises an Anti-Adrenomedullin antibody directed to the N-terminal end of Adrenomedullin comprising the following sequence: SEQ ID No. 1, Arginine in a concentration of 26.1 g/L, Trehalose in a concentration of 56.7 g/L, Poloxamer in a concentration of 0.5 g/L, Histidine in a concentration of 1.6 g/L and wherein said formulation exhibits a pH of 6,0.
12. Pharmaceutical aqueous formulation comprising an human or humanized anti-adrenomedullin (ADM) antibody or an anti-adrenomedullin antibody fragment according to any of embodiments 1 to 11, wherein said formulation is stable after storage for at least 4 weeks at 2 to 8° C. and wherein less than 5 wt/wt-%, preferably 4 wt/wt-%, more preferably 3 wt/wt-%, more preferably 2 wt/wt-%, more preferably 1 wt/wt-%, more preferably 0.05%-mol or most preferably 0.01 wt/wt-% of the total antibody is aggregated as measured by size exclusion High performance liquid chromatography (SEC-HPLC) and wherein said antibody is present at a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml.
13. Pharmaceutical aqueous formulation comprising a human or humanized anti-adrenomedullin (ADM) antibody or an anti-adrenomedullin antibody fragment according to any of embodiments 1 to 12, wherein said formulation is stable after storage for at least 4 weeks at 2 to 8° C. and wherein stable means that said formulation remains free from visible particles.
14. Pharmaceutical aqueous formulation comprising a human or humanized anti-adrenomedullin (ADM) antibody or an anti-adrenomedullin antibody fragment according to any of embodiments 1 to 13, wherein said formulation is stable after storage for at least 4 weeks at 2 to 8° C. and wherein stable means that said formulation remains free from subvisible particles.
15. Pharmaceutical aqueous formulation comprising an human or humanized anti-adrenomedullin (ADM) antibody or an anti-adrenomedullin antibody fragment according to any of embodiments 1 to 14, wherein the activity of said antibody or antibody fragment in said formulation is stable after stress and wherein stress is induced by storing said formulation for at least 4 weeks at 45° C., preferably 42° C., more preferably, 38° C., more preferably 36° C. and most preferably 40° C. and 85% rH, preferably 80 rH, preferably 70% rH, and most preferably 75% rH and/or by storing said formulation for at least 3 months of storage at 29° C., preferably 27° C., more preferably, 22° C., more preferably 20° C. and most preferably 25° C. and/or 70% rH, preferably 65% rH, preferably 55% rH, more preferably 50% rH and most preferably 60% rH and/or by storing said formulation for at least 6 months of storage at 9° C., preferably 7° C., more preferably, 2° C., more preferably 0° C. and most preferably 5° C. and/or by performing at least 1 Freeze/Thaw cycle with said formulation and/or by subjecting said formulation to mechanical stress comprising orbital shaking and overhead rotation and wherein the stability is determined by assessing the visual appearance comprising color, clarity and visible particles of said formulation after stress.
16. Pharmaceutical aqueous formulation comprising a human or humanized anti-adrenomedullin (ADM) antibody or an anti-adrenomedullin antibody fragment according to any of embodiments 1 to 15, wherein said antibody is present in a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml.
17. Pharmaceutical lyophilized formulation obtainable from a pharmaceutical aqueous formulation according to any of embodiments 1 to 16.
18. Method of making a ready-for-application solution comprising the steps:
   a. Providing a pharmaceutical aqueous formulation according to embodiments 1 to 16
   b. Adjusting the pharmaceutical aqueous formulation from step a) in a physiological acceptable solution by optionally diluting the volume of the pharmaceutical formulation from step a) with a physiological acceptable solution and by optionally aliquoting the pharmaceutical formulation from step a) wherein the diluted or adjusted aqueous pharmaceutical formulation is suitable for the administration in a patient.
19. Method of making a ready-for-application solution comprising the steps:
   a. Providing a lyophilized formulation obtained from the aqueous formulation according to any of embodiments 1 to 16 by freeze drying optionally without adding any other bulk reagent
   b. Reconstitution of the lyophilizate from step a) in water and/or a physiological acceptable solution for injection
   c. Adjusting the reconstituted formulation from step b) in a physiological acceptable solution by optionally diluting the volume of the reconstituted formulation from step b) with a physiological acceptable solution and by optionally aliquoting the reconstituted formulation from step b) wherein the diluted or adjusted aqueous pharmaceutical formulation is suitable for the administration in a patient.
20. Ready-for-application aqueous pharmaceutical formulation obtainable by a method according to embodiment 18 or 19 comprising said human or humanized anti-adrenomedullin (ADM) antibody or an anti-adrenomedullin antibody fragment in a dose of 1 to 10 mg/kg body weight.
21. Ready-for-application aqueous pharmaceutical formulation obtainable by a method according to embodiment 18 or 19 comprising said human or humanized anti-adrenomedullin (ADM) antibody or an anti-adrenomedullin antibody fragment in a dose of 2 to 8 mg/kg body weight, preferred 2 or 4 or 8 mg/kg body weight.
22. Pharmaceutical formulation according to any of embodiments 1 to 16, 20 and 21, for use in therapy or prevention of an acute disease or acute condition selected from the group comprising: SIRS, a severe infection, sepsis, shock selected from the group comprising shock due to hypovolemia, cardiogenic shock, obstructive shock and distributive shock, in particular cardiogenic shock, septic shock, shock due to Covid-19, shock due to burns and traumatic shock., acute vascular diseases as e.g. heart failure, congestion, in particular diuretic resistant congestion, inflammatory conditions, autoimmune diseases, metabolic diseases, brain diseases, cardiovascular diseases and drug-induced diseases symptoms of illness or an illness characterized by such symptoms, wherein the symptoms of illness are selected from the group of nausea, headache, muscle aches, back pain, shivering, and/or vomiting and migraine.
23. Pharmaceutical formulation according to any of embodiments 1 to 16, 20 and 21, for use in therapy or prevention of an acute disease or acute condition of a patient for prevention or reduction of organ dysfunction or prevention of organ failure in said patient, wherein said acute disease or acute condition is selected from the group comprising as e.g. severe infections, diabetes, cancer, acute and chronic vascular diseases as e.g. heart failure, myocardial infarction, stroke, atherosclerosis, shock and organ dysfunction, kidney dysfunction, liver dysfunction, burnings, surgery, traumata, poisoning, and damages induced by chemotherapy and wherein said disease is not SIRS, sepsis or septic shock.
24. Pharmaceutical formulation according to any of embodiments 1 to 16, 20 and 21, for use in therapy or prevention of an acute disease or acute condition of a patient for stabilizing the systemic circulation of said patient, wherein said patient suffers from a disease that is selected from the group comprising SIRS, sepsis, diabetes, cancer, acute vascular diseases as e.g. heart failure, shock as e.g. septic shock and organ dysfunction as e.g. kidney dysfunction.
25. Pharmaceutical formulation according to any of embodiments 1 to 16, 20 and 21,
   a) for use in therapy of an acute disease or acute condition of a patient for stabilizing the systemic circulation of said patient wherein said patient is in need of stabilizing the systemic circulation and exhibits a heart rate of >100 beats/min and/or <65 mm Hg mean arterial pressure and wherein stabilizing the systemic circulation means increasing the mean arterial pressure over 65 mmHg or
   b) for preventive use in therapy of an acute disease or acute condition of a patient in order to prevent that the heart rate increases to >100 beats/min and/or mean arterial pressure decreases to <65 mm Hg.
26. Pharmaceutical formulation according to any of embodiments 1 to 16, 20 and 21, for use in therapy or prevention of an acute disease or acute condition of a patient for the regulation of fluid balance, wherein said patient is a patient in need of regulating the fluid balance and suffers from a disease that is selected from the group comprising systemic inflammatory Response-Syndrome (SIRS), sepsis, diabetes, cancer, heart failure, shock, and kidney dysfunction
27. Pharmaceutical formulation according to any of embodiments 1 to 16, 20 and 21, for use in therapy or prevention of congestion in a patient wherein said patient has a disease or condition selected from the group comprising: congestive high blood pressure, swelling or water retention (edema), heart failure in particular acute heart failure, kidney, or liver disease.
28. Pharmaceutical formulation for use in therapy or prevention according to any of the embodiments 1 to 16, and 20 to 27, comprising:
   determining the level of a fragment of pre-pro-Adrenomedullin selected from the group comprising Midregional Proadrenomedullin (MR-proADM), C-terminal Proadrenomedullin (CT-proADM), Bio-ADM, ADM-gly, or Proadrenomedullin N-terminal 20 peptide (PAMP) or fragments thereof prior to drug administration in a bodily fluid obtained from said subject; and
   comparing said level of a fragment of pre-pro-Adrenomedullin selected from the group comprising MR-proADM, CT-proADM, PAMP, Bio-ADM or ADM-gly to a predefined threshold or to a previously determined level of said fragment, and
wherein for the correlation an elevated level of said fragment of pre-pro-Adrenomedullin selected from the group comprising MR-proADM, CT-proADM, PAMP, Bio-ADM or ADM-gly or fragments thereof above a certain threshold or above a previously determined level is used for patient stratification for a therapeutic use according to the present invention.
29. Pharmaceutical formulation for use in therapy or prevention according to any of the embodiments 1 to 16, 20 to 28 for use in therapy of a patient with shock, in particular septic shock, wherein said patient:
   has suffered from shock, in particular from a septic shock not longer than 8.4 hours at the starting point of treatment with said Anti-adrenomedullin (ADM) antibody or an anti-adrenomedullin antibody fragment and/or
   has been admitted to ICU not longer than 8.4 hours at the starting point of treatment with said Anti-adrenomedullin (ADM) antibody or an anti-adrenomedullin antibody fragment, and/or
   has not received organ support at all or not longer than 8.4 hours of organ support at the starting point of treatment with said Anti-adrenomedullin (ADM) antibody or an anti-adrenomedullin antibody fragment.
30. Pharmaceutical formulation for use in therapy or prevention according to any of the embodiments 1 to 17, 20 to 29 wherein said patient is characterized by having a level of dipeptidyl peptidase 3 (DPP3) in a sample of bodily fluid of said patient below a threshold prior to drug administration.
31. Pharmaceutical formulation for use in therapy or prevention according to embodiment 30, wherein said threshold of DPP3 in a sample of bodily fluid of said patient is between 20 and 120 ng/mL, more preferred between 30 and 80 ng/mL, even more preferred between 40 and 60 ng/mL, most preferred said threshold is 50 ng/mL.
32. Pharmaceutical formulation for use in therapy or prevention according to embodiment 30 or 31, wherein the level of DPP3 is determined by contacting said sample of bodily fluid with a capture binder that binds specifically to DPP3.
33. Pharmaceutical formulation for use in therapy or prevention according to embodiment 30 to 32, wherein either the level of DPP3 protein and/or the level of active DPP3 is determined and compared to a predetermined threshold.

### FIGURE DESCRIPTION

FIG. 1 A-C: Relative area [%] of Main Peak (A), HMWS (B) and LMWS (C) as determined by SEC-HPLC of unstressed and stressed anti-ADM antibody (HAM8101) samples. Graphs show mean value of two replicates measured each two times.

FIG. 2 A-C: Relative area [%] Main Peak (A), HMWS (B) and LMWS (C) as determined by SEC-HPLC of anti-ADM antibody (HAM8101) samples: untreated (t=0), freeze/thaw (F/T 5 cycles at −80° C./25° C.) and mechanical stress (overhead rotation and Orbital shaking). Graphs show mean value of two replicates measured each two times.

FIG. 3 A-C: Relative area [%] of Main Peak (A), Acidic Peaks (B) and Basic Peaks (C) as determined by CEX-HPLC of unstressed and stressed anti-ADM antibody (HAM8101) samples. Graphs show mean value of two replicates measured each two times.

FIG. 4 A-C: Relative area [%] of Main Peak (A), Acidic Peaks (B) and Basic Peaks (C) as determined by CEX-HPLC of anti-ADM antibody (Ham80101) samples: untreated (t=0), freeze/thaw (F/T 5 cycles at −80° C./25° C.) and mechanical stress (overhead rotation and Orbital shaking). Graphs show mean value of two replicates measured each two times.

FIG. 5: Results of DLS measurement of unstressed and stressed ant-ADM antibody (HAM8101) samples.

FIG. 6 A-B: Results of non-reduced CE SDS after accelerated Aging (A), Mechanical stress (B) and Freeze and Thaw show mean value and SD of two replicates measured each two times.

FIG. 7 A-B: Results of reduced CE SDS after accelerated Aging (A), Mechanical stress (B) and Freeze and Thaw show mean value and SD of two replicates measured each two times.

FIG. 8: DLS results of formulations 1-5 after storage at 40° C. and after mechanical stress.

FIG. 9: DLS results of formulations 6-11 after storage at 40° C. and after mechanical stress.

FIG. 10: DLS results of formulation 11-15 after storage at 40° C. and after mechanical stress.

FIG. 11: DLS results of formulation 16-21 after storage at 40° C. and after mechanical stress.

FIG. 12 A-B: Relative area [%] Monomer of SEC-HPLC of formulated anti-ADM antibody (HAM8101) stored for up to 2 weeks at 40° C.±2° C./75% rH±5% rH (A) and after mechanical stress and freeze & Thaw (B), graphs show mean.and SD of two replicates measured each twice.

FIG. 13 A-B: Relative area [%] HMWS of SEC-HPLC of formulated anti-ADM antibody (HAM8101) stored for up to 2 weeks at 40° C.±2° C./75% rH±5% rH (A) and after mechanical stress and freeze & Thaw (B), graphs show mean and SD of two replicates measured each twice.

FIG. 14A-B: Relative area [%] LMWS of SEC-HPLC of formulated anti-ADM antibody (HAM8101) stored for up to 2 weeks at 40° C.±2° C./75% rH±5% rH (A) and after mechanical stress and freeze & Thaw (B) graphs show mean and SD of two replicates measured each twice.

FIG. 15 A-D: Particle content below 5 μm (A), from 5 to 10 μm (B), above or equal to 10 μm (C) and above or equal to 25 μm (D) after 2 weeks of storage at 40° C.

FIG. 16 A-D: Subvisible Particle content below 5 μm (A), from 5 to 10 μm (B), above or equal to 10 μm (C) and above or equal to 25 μm (D) after mechanical stress.

FIG. 17 A-C: Relative area [%] Monomer (A), HMWS (B) and LMWS (C) content of SEC-HPLC of formulated anti-ADM antibody (HAM8101) stored for up to 4 weeks at 40° C.±2° C./75% rH±5% rH. Graphs shows mean and SD of two replicates measured each twice.

FIG. 18 A-C: Relative area [%] Monomer (A), HMWS (B) and LMWS (C) content of SEC-HPLC of formulated anti-ADM antibody (HAM8101) after mechanical stress and 5 Freeze & Thaw cycles. Graphs shows mean and SD of two replicates measured each twice.

FIG. 19: Relative Monomer content after external analysis via analytical ultracentrifugation.

FIG. 20 A-B: DLS results after storage at 40° C. (A) and after mechanical stress (B).

FIG. 21 A-D: Particles after thermal stress, Particle content below 5 μm (A), from 5 to 10 μm (B), above or equal to 10 μm (C) and above or equal to 25 μm (D) after 2 weeks of storage at 40° C.

FIG. 22 A-D: Particles after mechanical stress, Particle content below 5 μm (A), from 5 to 10 μm (B), above or equal to 10 μm (C) and above or equal to 25 μm (D) after mechanical stress.

FIG. 23 A-C: Relative area [%] of Main (A), acidic (B) and basic (C) species of CEX-HPLC of formulated anti-ADM antibody (HAM8101) stored for up to 3.5 weeks at 40° C.±2° C./75% rH±5% rH. Graphs show mean and SD of two replicates measured each twice.

FIG. 24 A-C: Relative area [%] of Main (A), acidic (B) and basic (C) species of CEX-HPLC of formulated anti-ADM antibody (HAM8101) after mechanical stress and 5 Freeze & Thaw cycles. Graphs show mean and SD of two replicates measured each twice

### EXAMPLES

### Example 1-Basic Characterization of an Anti-ADM Antibody (HAM8101): Methods

### 1. Materials

All excipients used for the formulation of HAM8101 were sourced in Unites States Pharmacopoeia (USP) and/or European Pharmacopeia (Ph. Eur.) compliant quality. All chemicals used for analytical methods were sourced in a quality adequate for the individual method (e.g., p.a.). Drug substance (DS) samples of HAM8101 in basic buffer and current formulation were provided by the manufacturer. All samples were stored at −80° C. with internal monitoring.

The water used throughout the project, except for HPLC analyses, was sterile water in WFI quality (AquaB. Braun water for injection, Braun, article number 0082479E).

### 2. pH Adjustment

The adjustment of the final pH of all formulations and buffers was performed with calibrated pH-electrodes (VWR) connected to a SevenExcellence pH-Meter (Mettler Toledo) at a temperature between 23° C. and 25° C. (compliant with Ph. Eur. and USP). The pH values were always adjusted to the desired target value±0.03. For pH measurements of formulated DS, samples were measured with calibrated pH-electrodes (Mettler Toledo) connected to a SevenExcellence pH-Meter (Mettler Toledo) at a temperature between 23° C. and 25° C. (compliant with Ph. Eur. and USP).

### 3. Determination of Osmolality

Determination of osmolality was performed by freezing point depression in an Osmometer (Osmomat 030-D RS, Gonotec). Each measurement was performed in 15 μl aliquots after calibration with sodium chloride standard (600 mOsmol/kg) and purified water.

### 4. Dialysis for Buffer Exchange

The buffer exchange of the DS for re-formulation was performed by dialysis in Slide-A-Lyzer Cassettes (Thermo) with a nominal cut-off of 20 kDa. Samples were dialyzed three times each for at least 2 hours slowly stirred at 2-8° C. in a freezer, from which one step was performed overnight (>12 h). After dialysis, HAM8101 concentration was adjusted to the target value 20 g/L ±2 g/L with corresponding buffer, if necessary.

### 5. Concentration Measurements

The concentration of the drug substance was measured by absorption at 280 nm using the Spark Plate Reader (Tecan, Schweiz). To do so, placebo and DS samples were measured in one 96 well plate under the same conditions. Then, adsorption of placebo was subtracted from corresponding DS samples.

### 6. Vial Filling

Before vial filling, samples were sterile filtered with 0.1 μm PES-filters. For liquid storage in step 3 and 4, a volume of 2 ml of each formulation with 20 mg/ml ±2 mg/mL DS were inserted into sterile, particle-free 2R-vials (provided by Rentschler) under a laminar air flow. he vials were closed with sterile stoppers (provided by Rentschler) and crimped with suitable aluminum caps.

### 7. Sample Storage

The samples stored at 5±3° C. were kept in refrigerators with external temperature monitoring. DS in original formulation was stored at −80° C. in a freezer with internal monitoring of temperature.

Samples subjected to an accelerated aging were stored in ICH cabinets (Memmert) with controlled humidity at 25±2° C./60±5% rH, 30±2° C./65±5% rH and 40±2° C./75±5% rH, respectively. The temperature was additionally monitored independently during the complete storage period. Freeze/thaw stress was performed in cooling cells with alternating storages at −80° C. and +25° C., for at least 2 hours each. The freezing and thawing result was visually controlled and adapted accordingly. Shear stress was applied by over-head rotation (Over-Head shaker, Hei-Mix Reax 2, Heidolph/VWR) at 30 rpm for 24 h or orbital shaking (Dual Plate Shaker PSU-2T, Kisker) at 400 rpm for the indicated time periods, at uncontrolled room temperature. For all storage time points for up to one month, samples were pulled within +/−1 day of target pull date and for two, three- and six-months stability duration within +/−3 days from the target pull date.

### 8. Size Exclusion Chromatography (SE-HLPC)

SE-HPLC method was based on the SOP provided by the manufacturer for HAM8101. In the size exclusion chromatography method, the stored samples (20±2 mg/ml HAM8101) were separated on a TSKgel G3000SWXL column (5 μm, 7.8×300 mm, Tosoh Bioscience, No: 0008541) using a Thermo Ultimate 3000 HPLC system with an isocratic flow of 0.5 ml/min at 20±5° C. (50 mM Sodium phosphate, 300 mM Sodium chloride, pH 7.0). Data were recorded at 214 and 280 nm by UV detector. Per injection, 20 μg DS diluted in 20 μl mobile phase were loaded. Data analysis was performed with Chromeleon 7.2.6.10049 software. For column performance check a molecular weight marker (1511901; Bio-Rad) was used prior to and after every set of reference standard and 10 samples, comprising thyroglobulin, γ-globulin, ovalbumin, myoglobin, and vitamin B12. To prevent high pressure, up to Example 3 (WP4a) all turbid samples (e.g., FT samples, F01 and F06) and from Example 4 (WP4b) onwards all samples and all placebos were filtered with 0.1 PES filters (Sartorius, 16553).

### 9. Cation Exchange Chromatography (CEX-HLPC)

CEX-HPLC method was based on the SOP provided by Rentschler for HAM8101. In the cation exchange chromatography method, the stored samples (20±2 mg/ml HAM8101) were separated on a MAbPac SCX-10 column (5 μm, 4 mm×150 mm; Thermo Scientific, No.: 085198) on a Thermo Ultimate 3000 HPLC system. A gradient (see Table 1) with Diluent A: 100% 20 mM 2-(N-morpholino) ethanesulfonic acid (MES) and Diluent B: 100% 20 mM MES with 200 mM sodium chloride with pH 6.0 over 40 minutes was used to separate the antibody charged variants at a constant flow rate of 0.5 ml/min at 25±5° C. Per injection, 60 μg DS diluted in Eluent A in 30 μl were loaded. Data were recorded at 280 nm and 214 nm by UV detector. Data analysis was performed with Chromeleon 7.2.6.10049 software.

To prevent high pressure, up to Example 3 (WP4a) all turbid samples (e.g., FT and 2w 40° C. samples, F01 and F06) and from Example 4 (WP4b) onwards all samples and all placebos were filtered with 0.1 PES filters (Sartorius, 16553).

### 10. CE-SDS/Red./Non-Red)

Rentschler provided a CE-SDS method SOP with attachments for the analysis of HAM8101 for quantitative determination of low molecular weight species (LMWS) by means of non-reducing CE-SDS analysis. However, it was agreed to perform CE-SDS on all samples—reduced as well as non-reducing—during the course of this project. Therefore, standard instrument settings according to the instrument supplier recommendations were used, as well as standard sample buffer (pH 6.8) for CE-SDS. Samples were diluted with water to a concentration of 3 mg/mL but were not subsequently concentrated via zentricons as described in the manufacturer's SOP. The eluent was prepared according to the SOP, the samples were diluted, and the sequence runs were also performed corresponding to the SOP but the results with sample buffer pH 6.8 were not comparable to the results provided by the manufacturer. Thus, samples were diluted with sample buffer pH 9.0 and the sequence run was repeated. Using the pH 9.0 sample buffer, the results were comparable, and the instrument settings and sample preparation was used for further WPs of this project

### 11. Dynamic Light Scattering (DLS) for Aggregate Testing

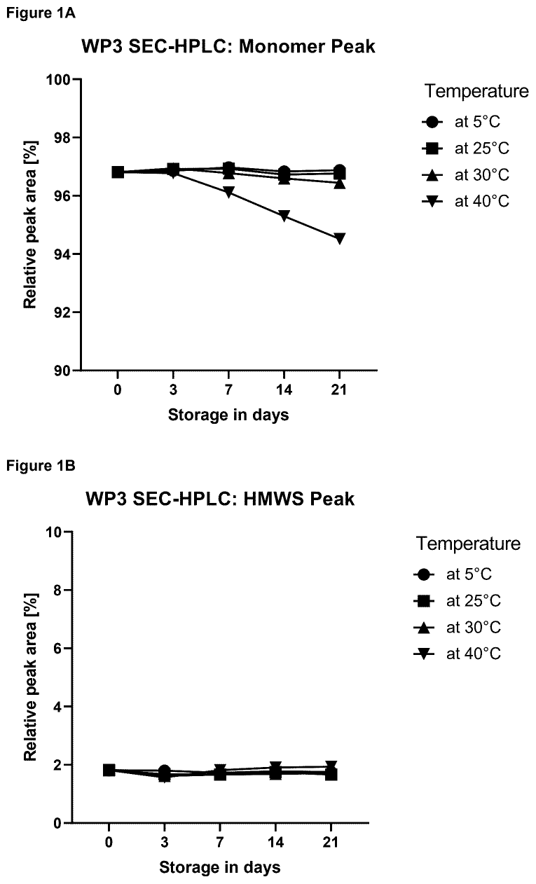

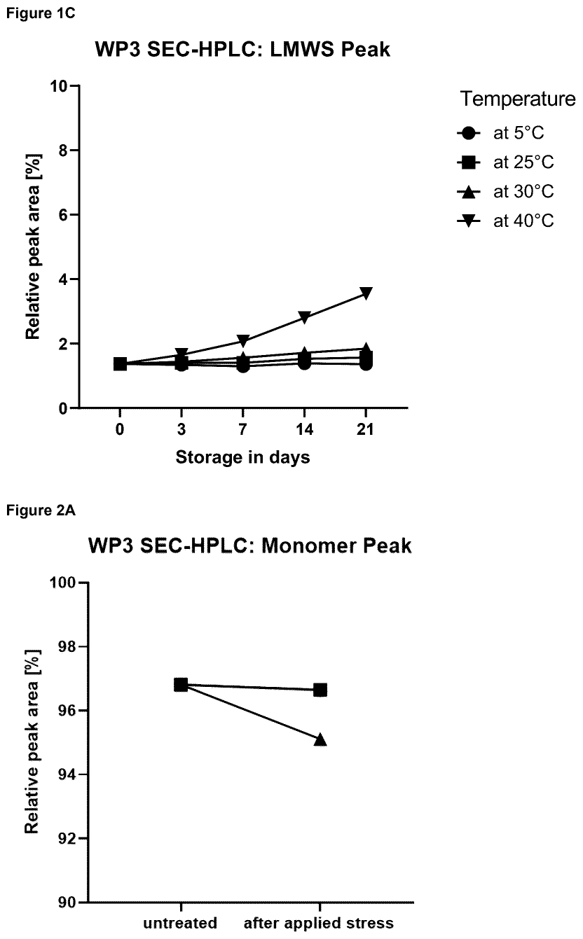

Dynamic light scattering was performed in a DynaPro Plate Reader III (Wyatt) in 384 well plates (Aurora) at 25° C. For implementation of the method, samples were measured undiluted with 20 mg/ml and diluted to different concentrations (0.25 mg/mL, 0.5 mg/mL, 0.75 mg/mL and 1 mg/mL) with placebo measured alongside the samples. All subsequent measurements were performed with 35 μL of undiluted samples and 35 μL of all placebos measured under the same conditions. After sample filling, the plates were sealed with sealing tape and shortly centrifuged before being put into the DLS Plate Reader at 25° C. Analysis was performed with Dynamics, version 7.9.14. For each replicate, 10 individual acquisitions for 5 s each were recorded, as indicated in FIG. 1.

Results are reported as mean value of two biological replicates with three technical replicates each. All measurements were reported as intensity weighted results. In DLS, particles having hydrodynamic radii in the nanometer scale are monitored. Degradation products can be either fragments with smaller radii than the radius of the monomer DS or aggregates with higher radii that each appear as additional population. The results can be visualized as e.g., histograms of particle size populations. Category type plots of DLS peaks containing D50-values and peak radii in combination with a histogram-based intensity weighting were applied. For the stability study, DLS data were presented as D50-values over radius scatter plots of all measured peaks with intensity weighted symbol size in order to allow for easier comparison of the different formulations during storage time. The radii for each population (x-axis) and the D50 value of all particles measured (y-axis) of each measurement are plotted within a log-log scatter plot.

The depicted symbol size of each peak is proportional to the relative intensity of the respective peak (% Intensity weighted). The overlay of replicate measurements using transparent color in addition enables the evaluation of the reproducibility of each signal by color intensity being useful in main peak determination. Unless otherwise described, each measurement consists of at least 2×100 single measurements. Parameters derive from a volume distribution and from an intensity distribution, which is itself obtained from a correlation function. Overall, the D50 value over peak radius plots with intensity weighted symbol size are used for qualitative comparison of the formulations at the applied time points within the different study arms of the stability study.

### 12. Turbidity

Turbidity of samples was analyzed by nephelometry or by measuring the absorption at 350 nm, 510 nm and 550 nm. Turbidity by absorption at 350 nm, 510 nm and 550 nm was measured using the Spark Plate Reader (Tecan, Schweiz). To do so, placebo and DS samples were measured in one 96 well plate under the same conditions. If enough sample volume (minimum 1.5 mL) was available, turbidity of formulated drug substance was measured in cuvettes (HACH-Lange) with an inner diameter of 11 mm on a calibrated turbidimeter LAB 2100AN (HACH-Lange) by comparing the measured turbidity to standards with known turbidity (<0.1, 3, 6, 18, 20 and 30 NTU, acc. to European Pharmacopeia 6.0 as described in the StablCal®-Kit user manual; HACH-Lange).

### 13. Subvisible Particles (SVP)

Micron-sized protein aggregates and particles (subvisible particles, SVP) are important quality attributes of therapeutic protein formulations due to their risk of enhancing an immunogenic response. Quantification of subvisible particles larger than 10 μm and 25 μm is therefore required by the pharmacopoeias and is commonly performed using light obscuration (LO) techniques. Currently, the acceptance criteria for SVP are 6000 NMT≥10 μm/container and 600 NMT≥25 μm/container. SVPs<10 μm have to be monitored, acceptance criteria are not defined. However, quantification and characterization of particles with a size below 10 μm is of increasing interest and are in the meanwhile a regulatory expectation. In this study, measurements were performed by microfluid imaging using a FlowCam (Fluid Imaging Technologies) according to the Software settings below. The software visual spreadsheet (version 4.12.3, Fluid Imaging Technologies) was used for analysis. The data were size grouped acc. to Ph. Eur./USP into subvisible particles (SVPs) below 5 μm, 5-10 μm, above or equal to 10 μm and above or equal to 25 μm. Sample and placebo measurements were performed undiluted. After every technical replicate at least one cleaning and washing step with 4% Hellmanex III (Sigma-Aldrich) and water was performed. Only if a SST measurement with water exhibited no particle pollution, the sample measurement was performed.

It has to be emphasized, that according to literature, MFI is supposed to deliver higher particle counts if compared to LO due to a higher sensitivity for particles with a refractive index similar to the solvent Huang et al., 2009). This especially applies to proteinaceous particles.

### 14. Visual Color Inspection

Appearance testing of formulated DS was performed regarding color according to Ph. Eur. method 2.2.2. Visible particles, (Ph. Eur. method 2.9.20) clarity and degree of opalescence (Ph. Eur. method 2.2.) were analyzed according to Ph. Eur. method 2.9.20 and 2.2.1 respectively. Above assessments were performed using an inspection light box equipped with non-flickering fluorescent lamps and a black and a white background plate. For color assessment, samples were compared to Ph. Eur. reference solutions and freshly prepared water. The samples were evaluated for 5 s without magnification to assess number and shape of visual particles and clarity in front of black and white background.

### 15. Analytical Ultracentrifugation

Chosen samples analyzed via analytical ultracentrifugation (AUC) were sent to external analysis. At Zentriforce Pharma Research GmbH, the sample's sedimentation profile is detected by an optical detection system. Thus, the shape, conformational change and size distribution of molecules can be determined.

### Example 2: Basic Characterization of an Anti-ADM Antibody (HAM8101)

The response of an anti-ADM antibody (HAM8101) in current buffer (20 mM His/HCL, 300 mM Glycine, pH 6.0) to different stress modes was investigated in forced degradation studies comprising temperature, mechanical and freeze/thaw stress as summarized in Table 2 below. Samples were tested by SE-HPLC, CEX-HPLC/CiEF, CE-SDS (red/non-red), Appearance, Nephelometry, DLS and SvP after 3, 7, 14 and 21 days of storage.

### 1. Size Exclusion Chromatography (SE-HPLC)

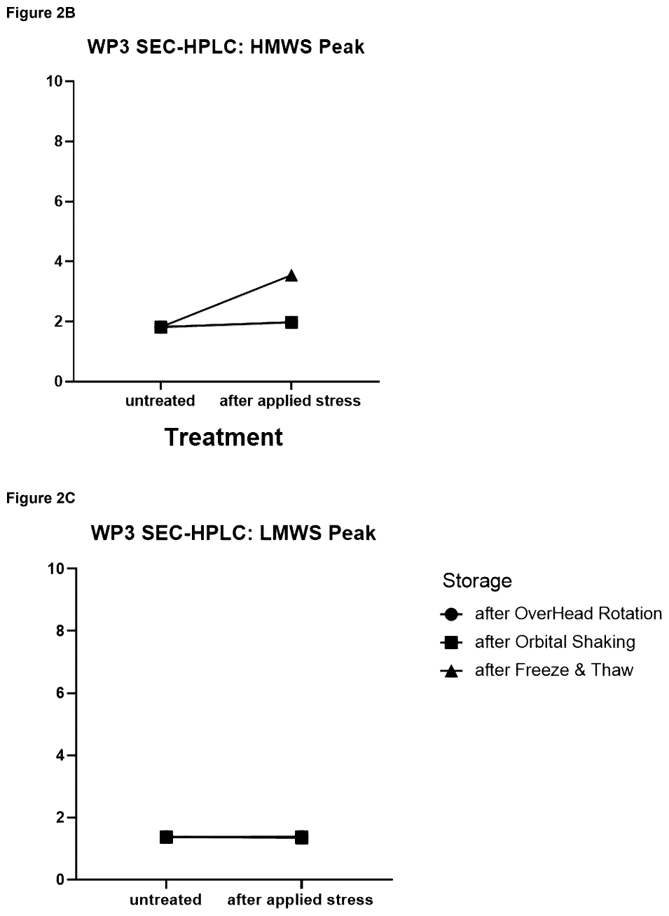

Results of SEC-HPLC were analyzed as relative peak area [%], mean values of two replicates measured each two times were calculated and displayed in FIG. 1. In addition to the storage at elevated temperature, the samples were stressed by overhead rotation, Orbital shaking at room temperature and 5 freeze/thaw (F/T) cycles. The SEC-HPLC results of mechanical and freeze/thaw stress are shown in FIG. 2. The key results of the SEC Analysis are: 1. Temperature dependent increase of LMWS during thermal storage (strongest increase at 40° C.) 2. Increase of HMWS in SEC after 5 F&T cycles:

### 2. Cation Exchange Chromatography (CEX-HPLC)

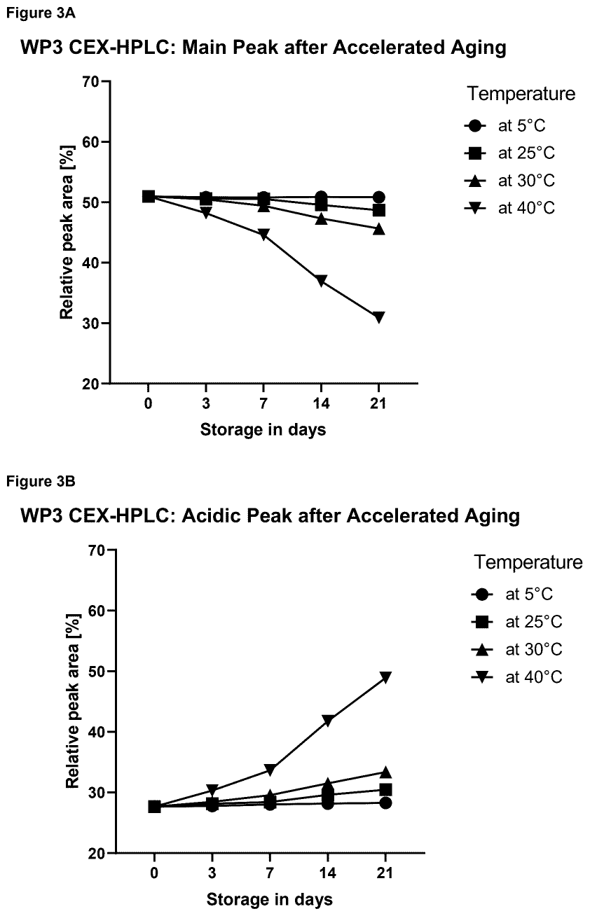

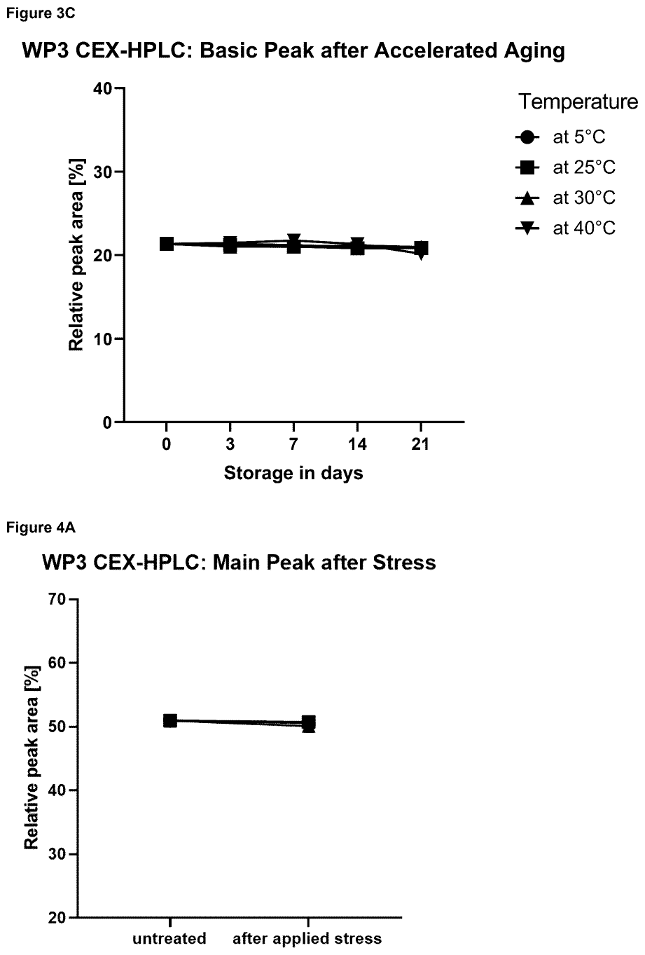

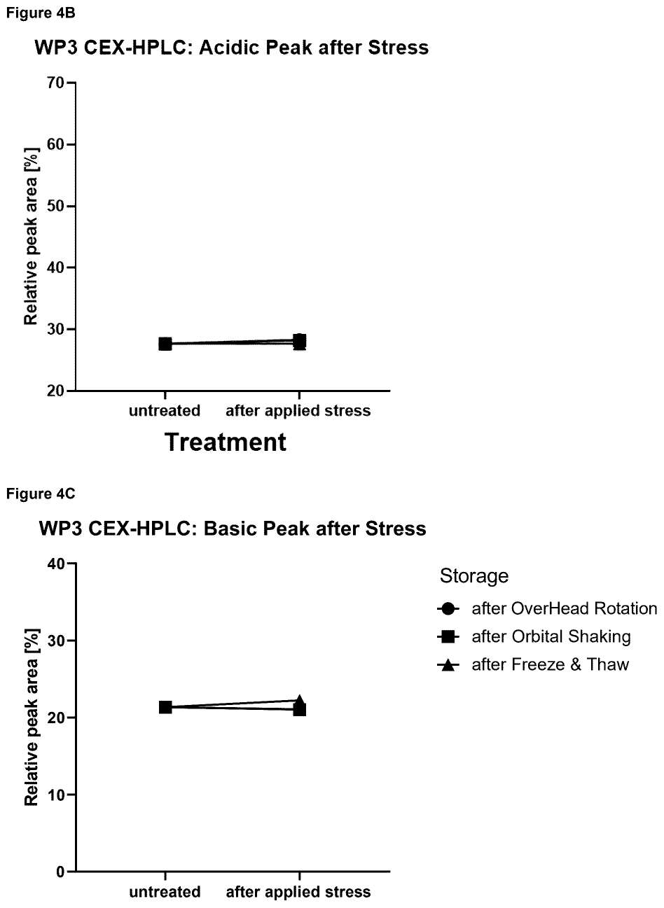

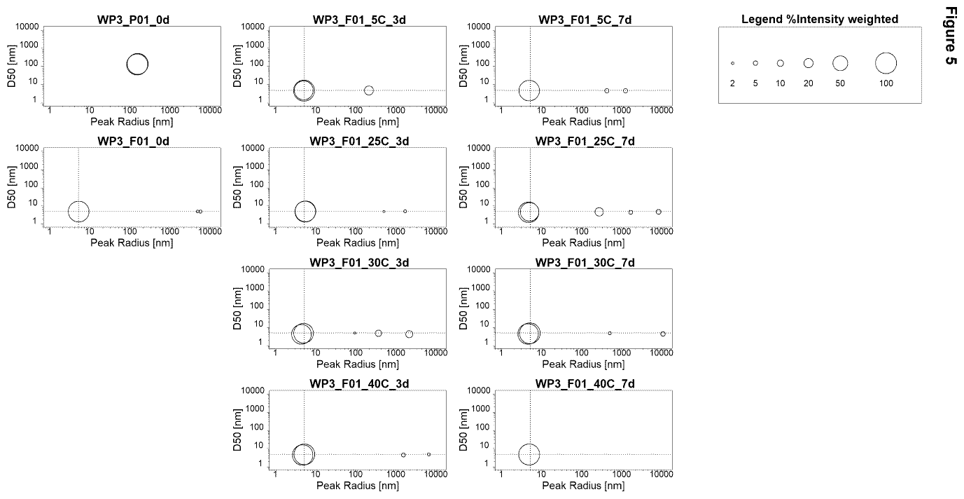

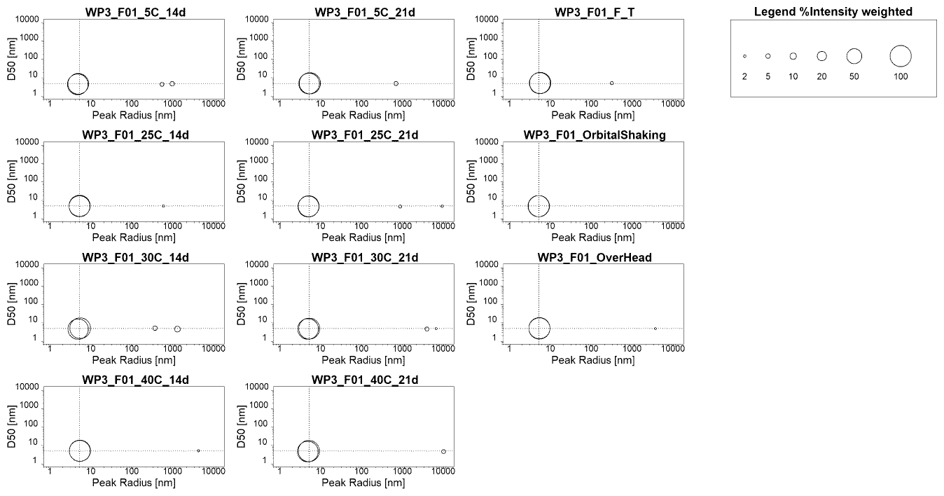

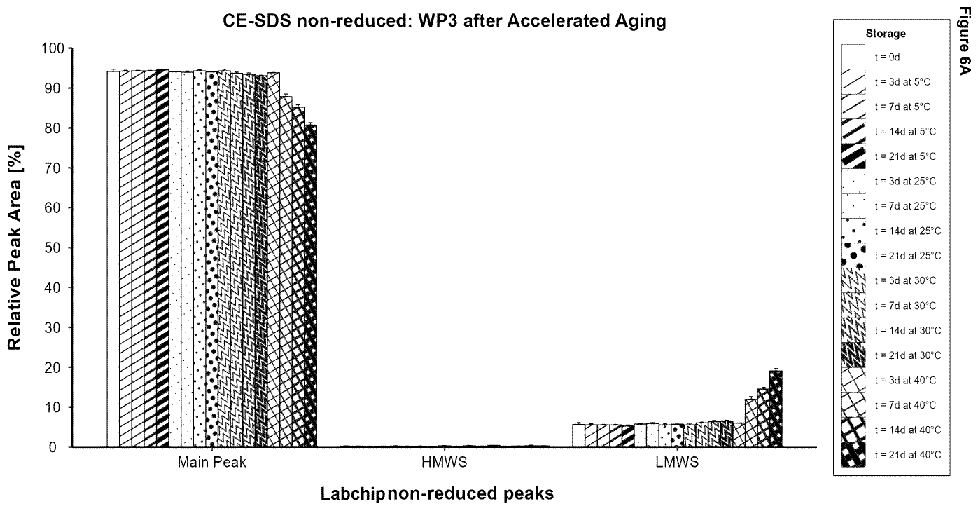

Results of CEX-HPLC were expressed as mean relative peak areas derived from two replicates measured in duplicates. The results are shown in FIG. 3. In addition to the storage at elevated temperature, the samples were stressed by overhead rotation, Orbital shaking at room temperature and 5 freeze/thaw (F/T) cycles. These results are depicted in FIG. 4. The CEX analysis leading to following conclusions: CEX-HPLC revealed a significant rise of acidic species during storage, in particular in the samples stored at 40° C. A possible explanation would be deamidation.

### 3. Dynamic Light Scattering (DLS)

The results of the DLS analysis of the original formulation are shown in FIG. 5. Shift of blue dots to right (higher particle size of certain populations) and top (higher mean particle size) indicates aggregation. Shift to left indicates fragmentation. High number of dots indicates that a sample contains various population of different particle size, i.e., hydrodynamic radius; plots show mean of 2 technical replicates, for each replicate 3×100 acquisitions a is were evaluated. Overall, there was no distinct effect of stressing visible. In all samples higher particle sizes are detectable, which was expected.

### 4. Appearance

The results of the visual appearance after stress and subsequent freezing at −80° C. (Table 3), followed by aliquotation and another cycle of freezing at −80° C. (Table 4) are shown below. Notably, the visible particle burden after overnight incubation at 5° C. seemed to be less than the day before, a few hours after thawing. Therefore, freeze & thaw stress seems to be critical.

### 5. CE-SDS (Red./Non-Red)

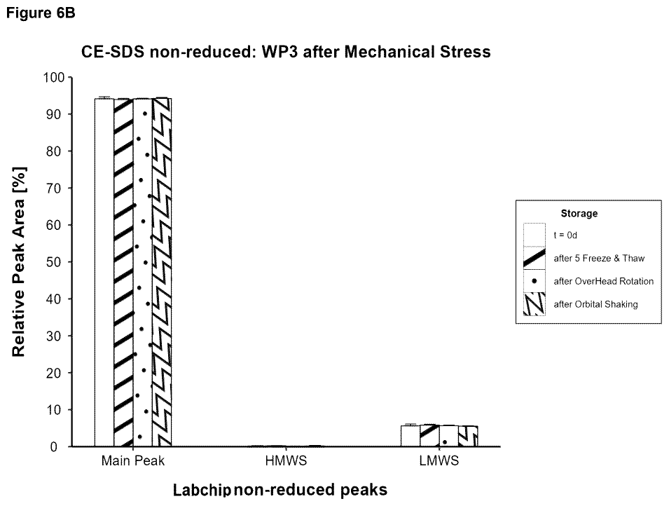

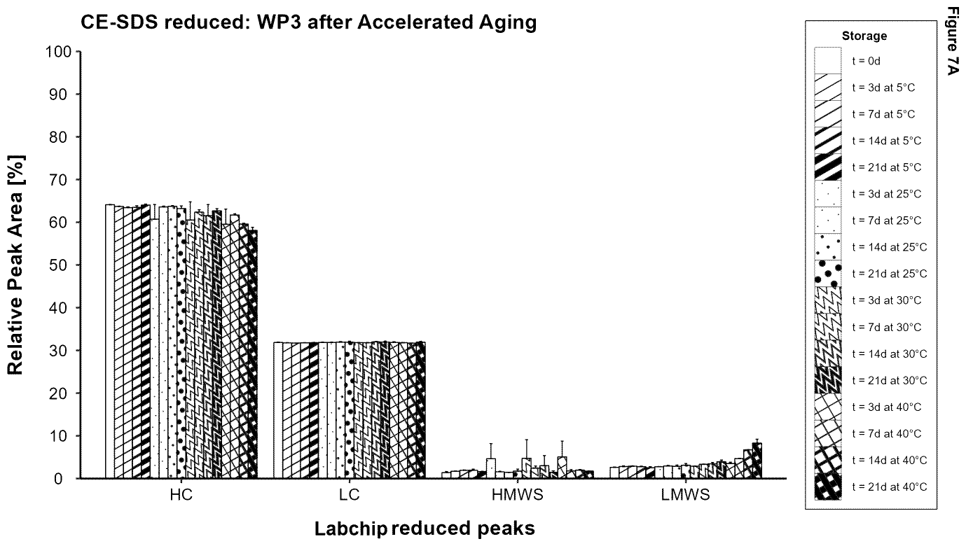

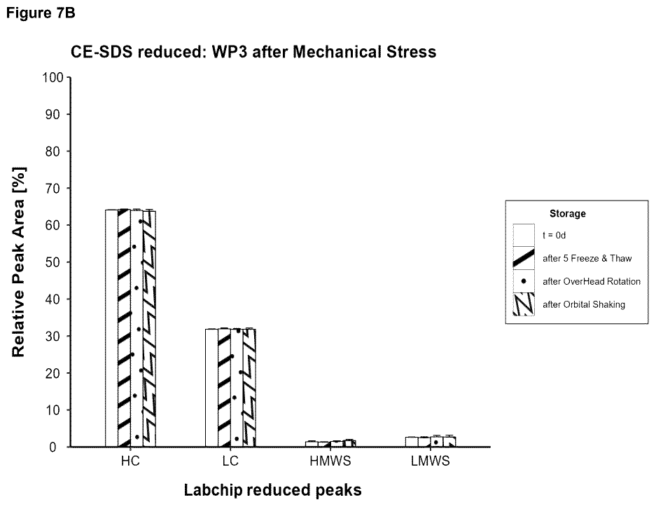

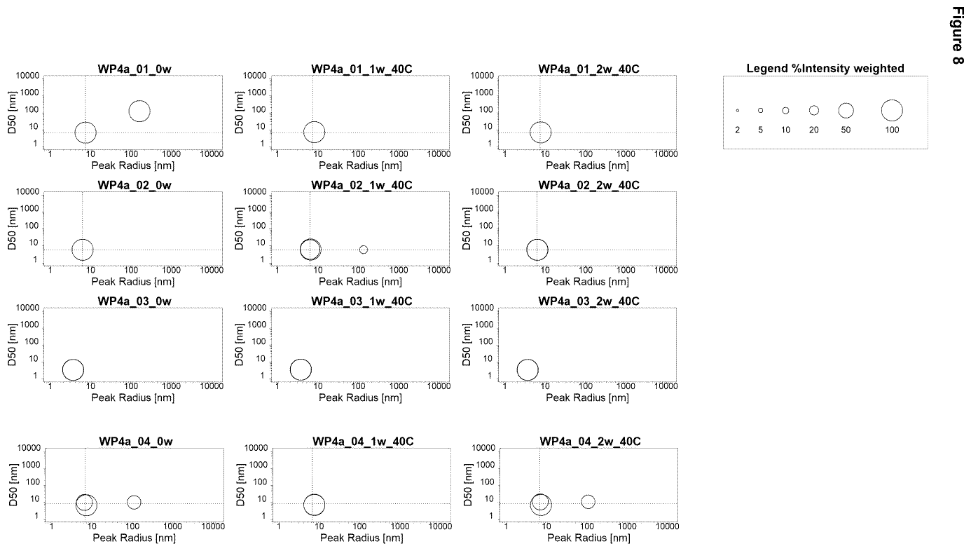

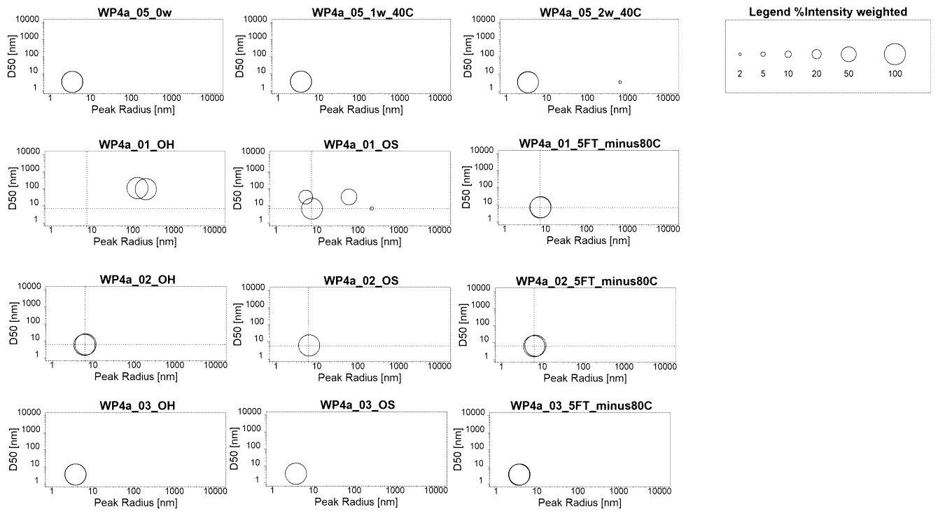

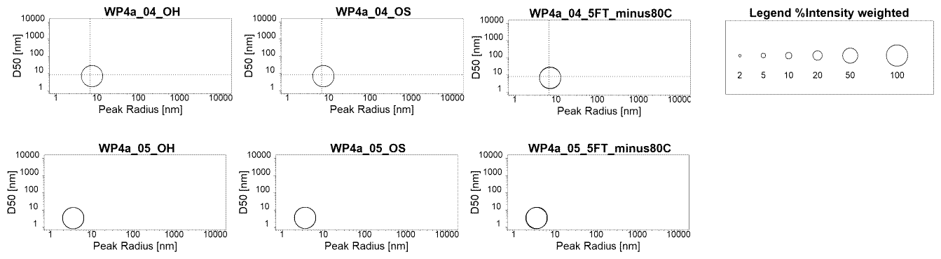

Results of CE-SDS were expressed as mean relative peak areas derived from two replicates measured in duplicates (see FIGS. 6 and 7). The key results of the CE-SDS Analysis are: 1. Rising LMWS at 40° C. after 7 days and beyond 2. No effect of mechanical stress detected 3. CE-SDS data fit to the SEC data

### Example 3: Design, Test and Optimization of Formulations to Stabilize an Anti-ADM Antibody (HAM8101) and Reduce Particle Formation in Liquid Formulation During Two Rounds of Accelerated Aging-Screening DoE Design

Here, the implementation of the first accelerated aging round and the respective results are presented. The purpose of this screening round was to investigate the influence of the different parameters of screening DoE, as shown in Table 5. The reference of anti-ADM antibody (HAM8101) was formulated in current buffer (20 mM His/HCL, 300 mM Glycine, pH 6.0).

The following stress conditions were chosen based on the results as shown in Example 2:
2 weeks of storage at 40° C.±2° C./75±5% rH
Freeze/Thaw cycles 5× (−80° C./+25° C.) (1° C./min; Plateau 2h)
Mechanical stress: Orbital shaking and overhead rotation as described above.

Analytical methods employed are shown in Table 2, while as the composition of the different formulations is shown in Table 6.

### 1. Osmolality and Concentration

First, the osmolality and concentration of samples are described. The measured data are shown in Table 7.

### 2. Dynamic Light Scattering (DLS)

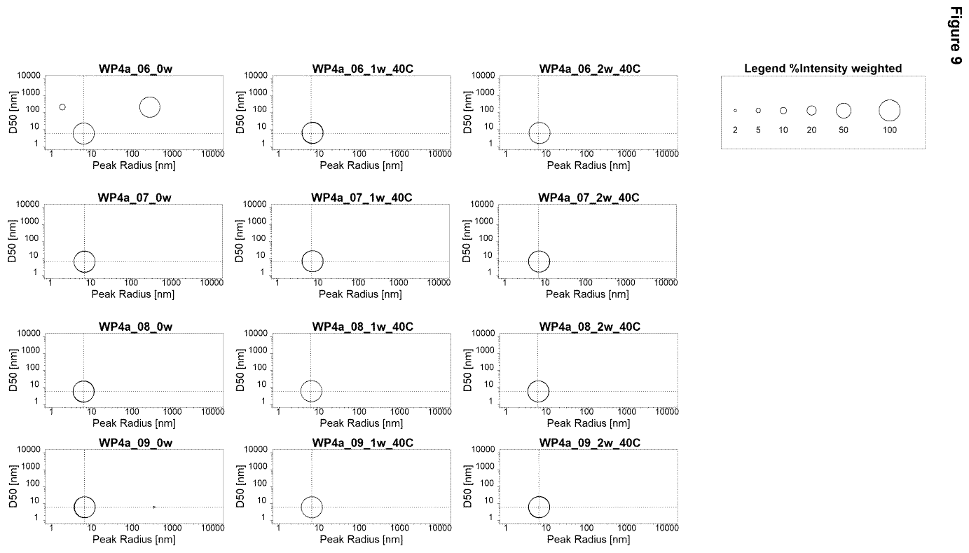

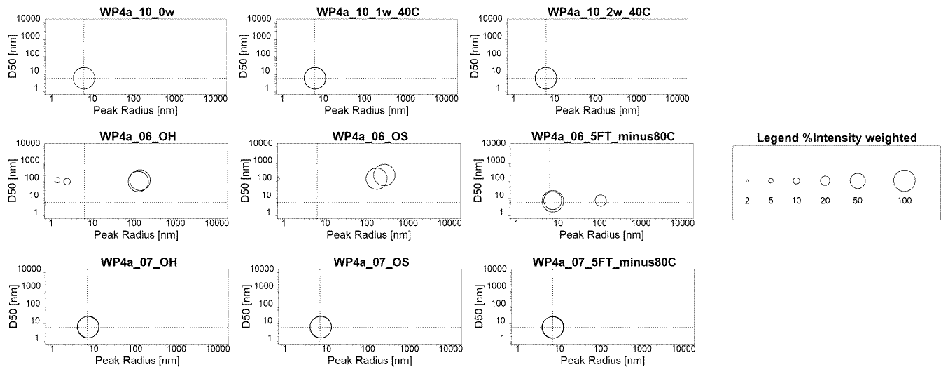

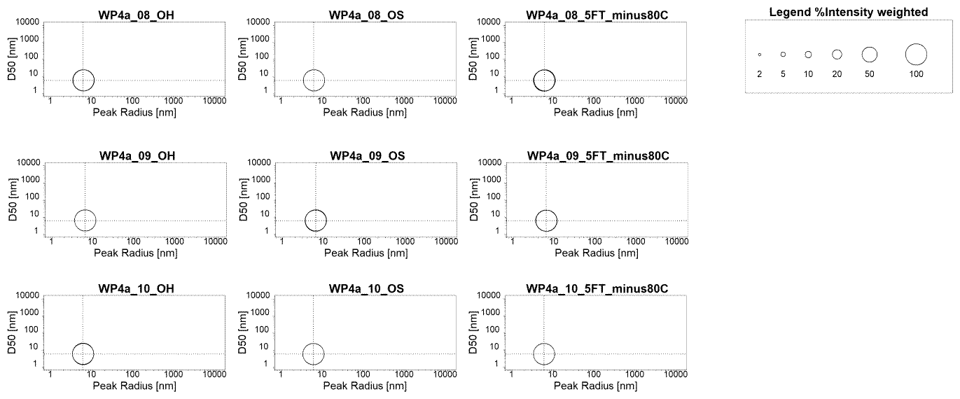

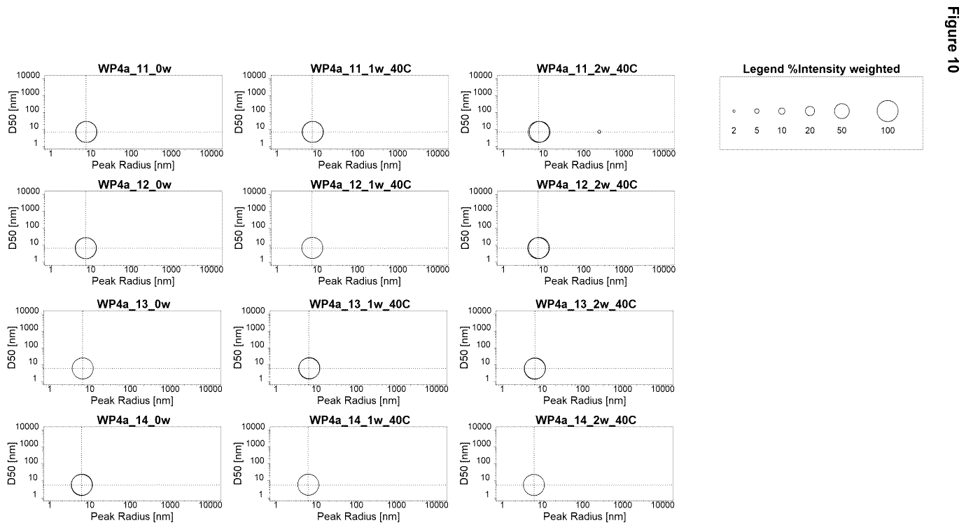

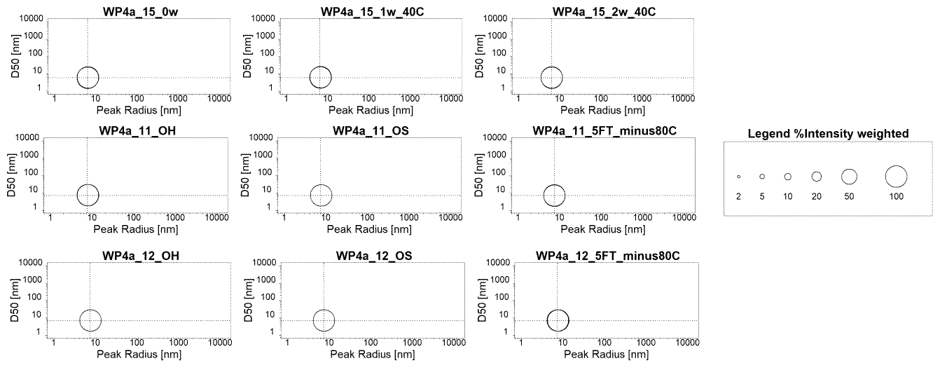

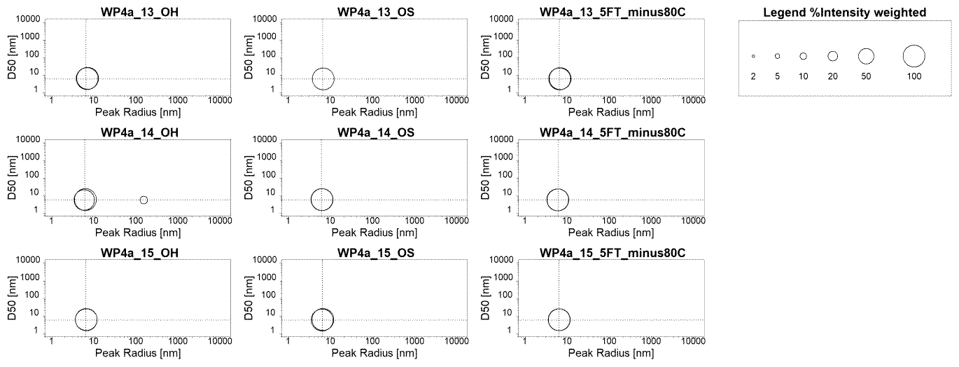

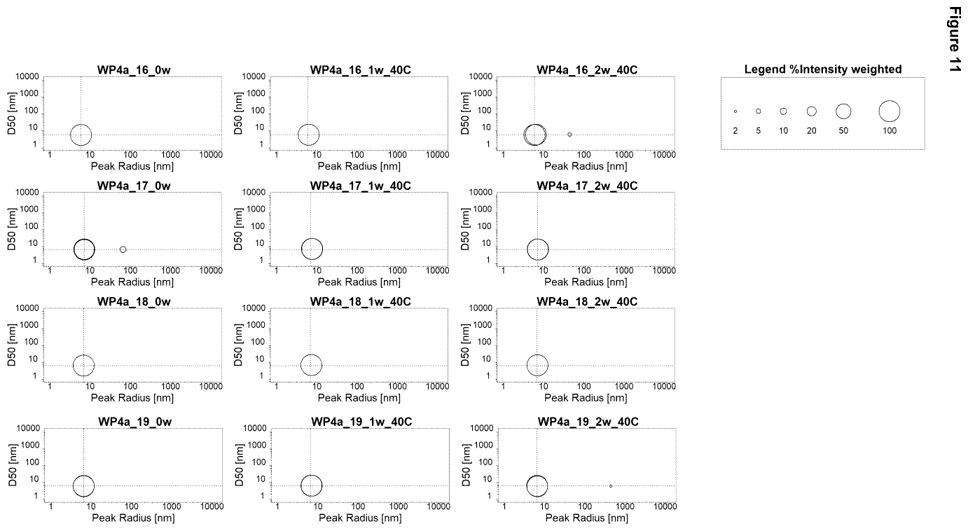

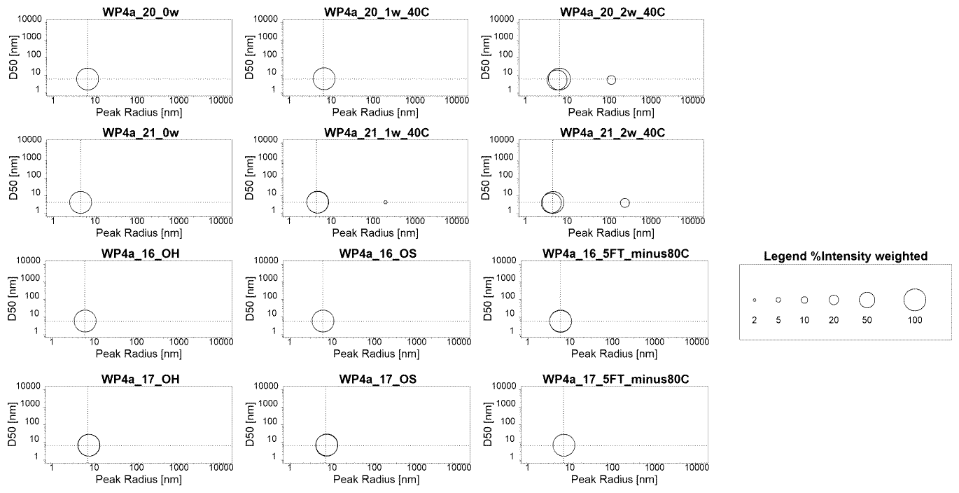

The results of the DLS analysis of the WP4a formulation are shown in FIG. 8-FIG. 11. The results indicated a very high sensitivity for large radii (≤×6) to see early changes and the D50 value (˜5 nm) confirmed that most particles are in size range of the API.

### 3. Size Exclusion HPLC

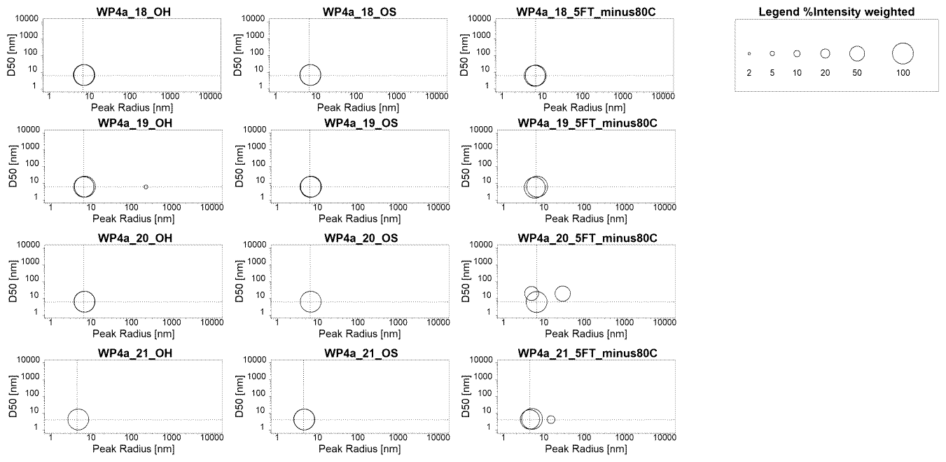

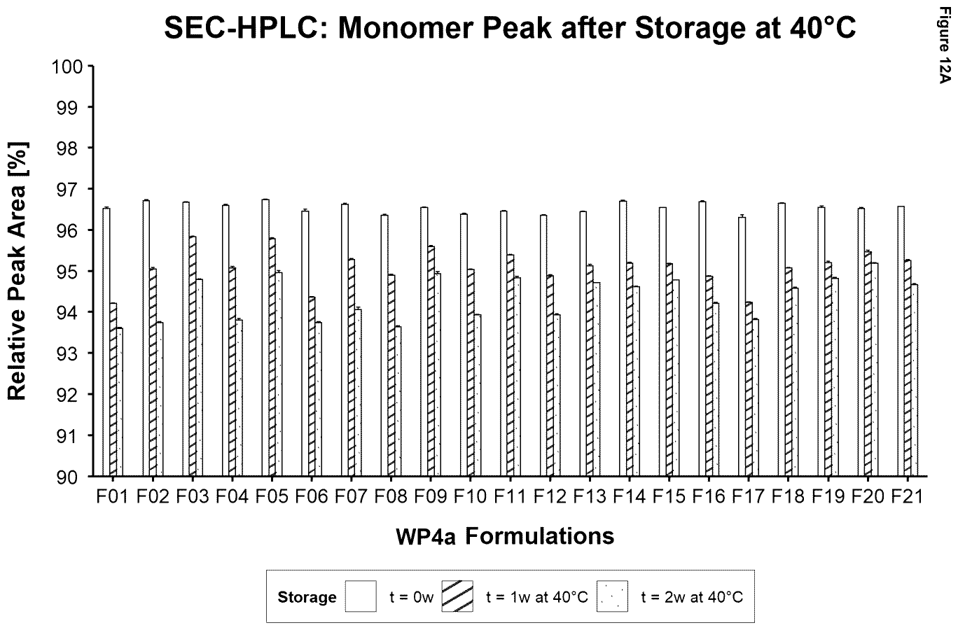

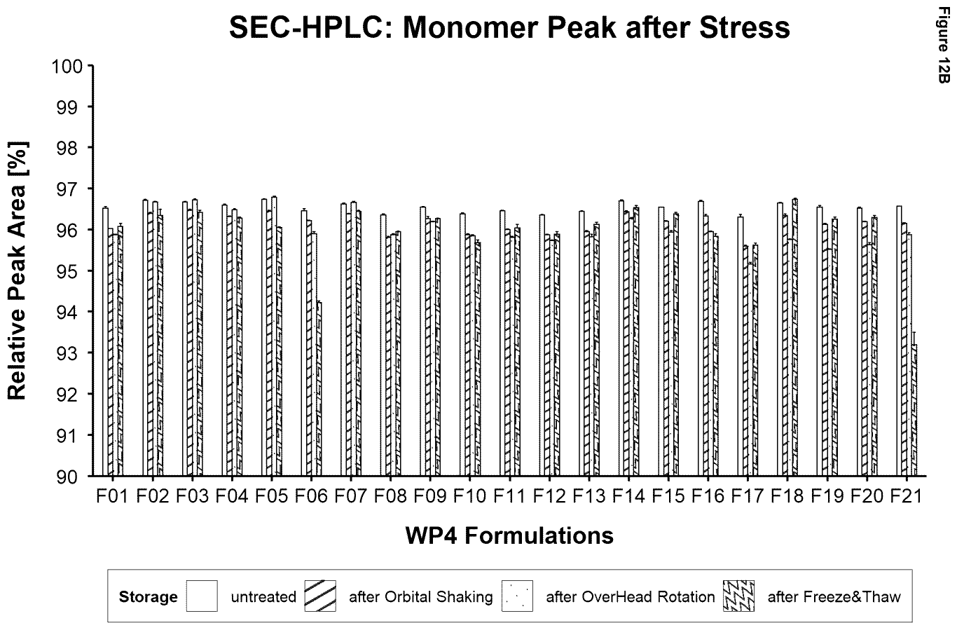

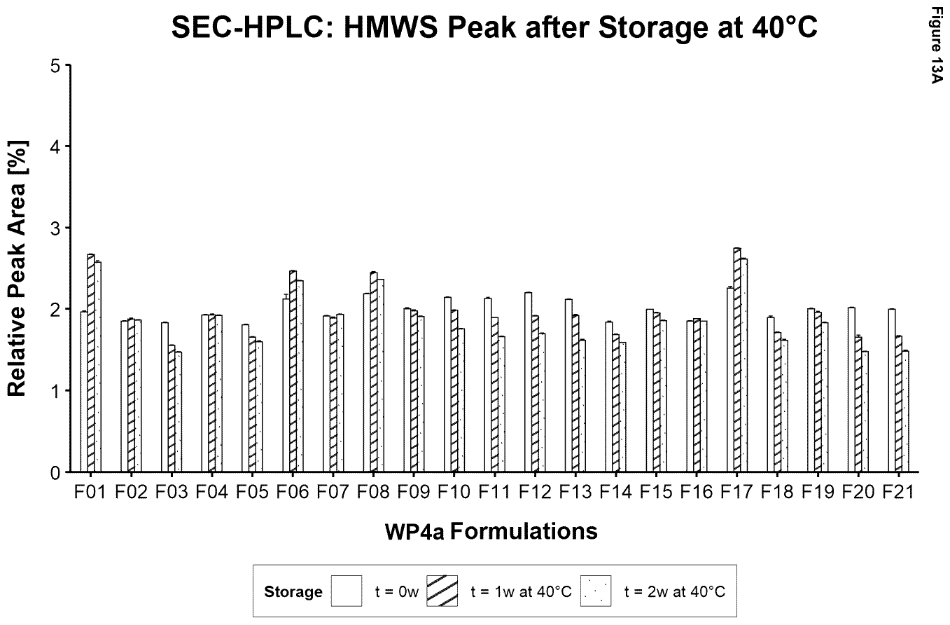

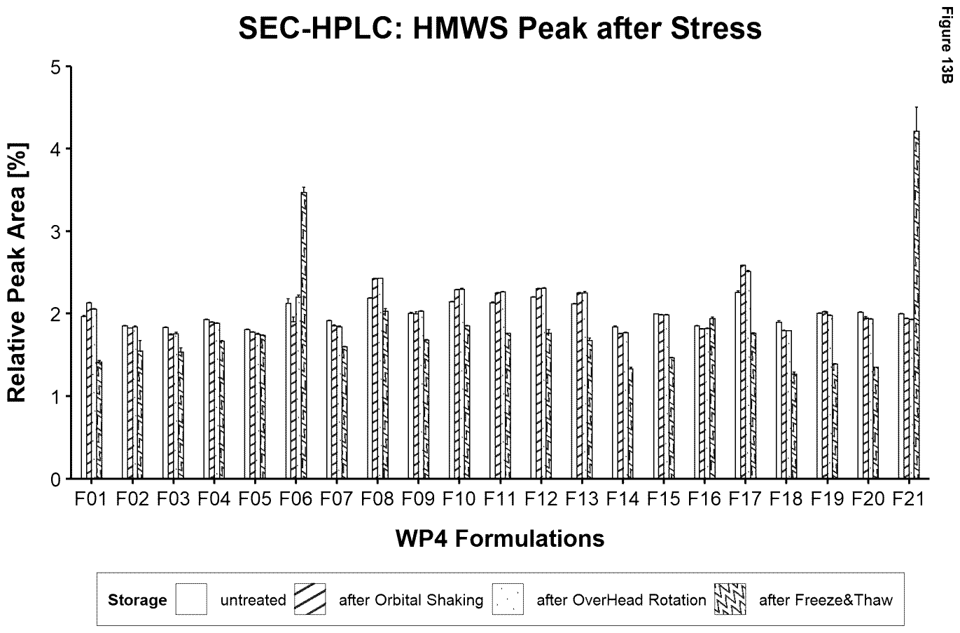

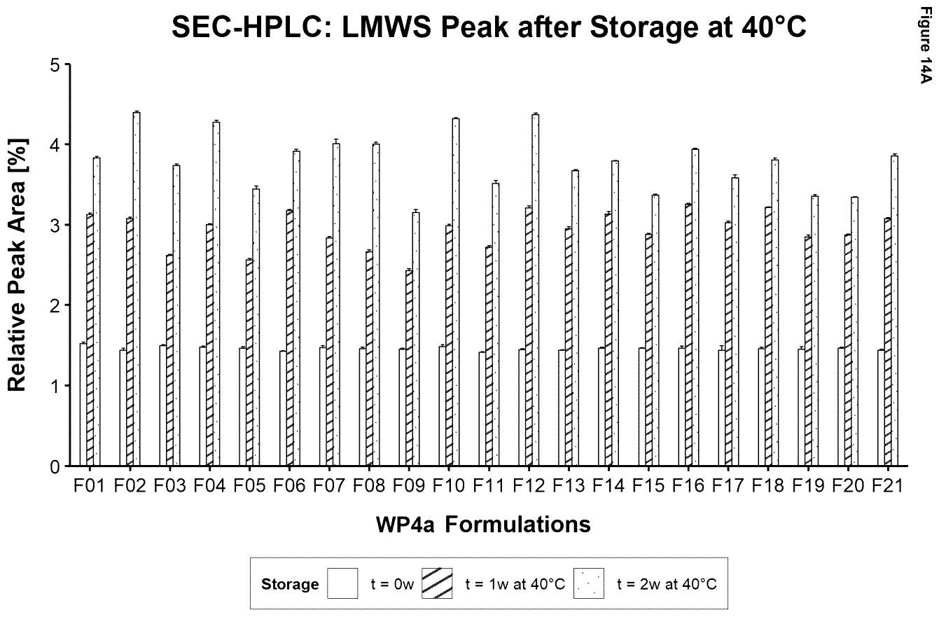

Results of SEC-HPLC were expressed as mean relative peak area [%], derived from two replicates measured in duplicates and displayed in FIGS. 12 to 14.

### 4. Subvisible Particles (SVP)

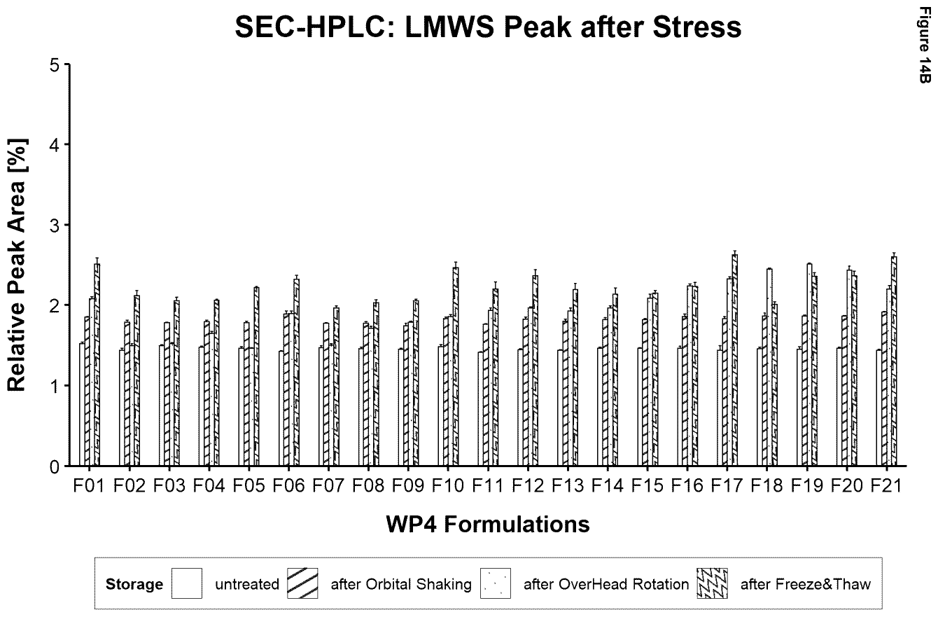

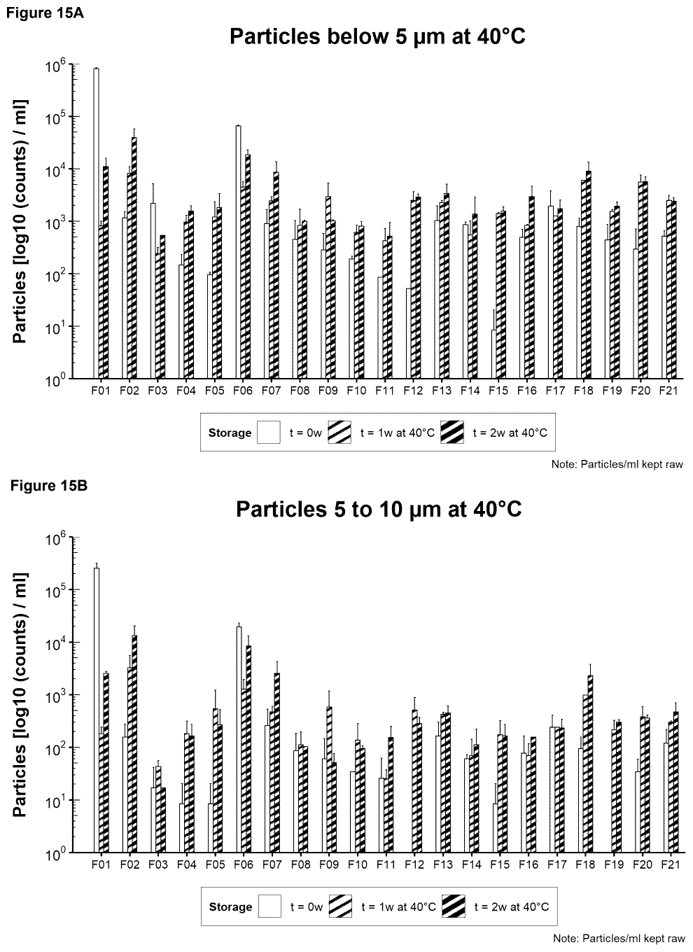

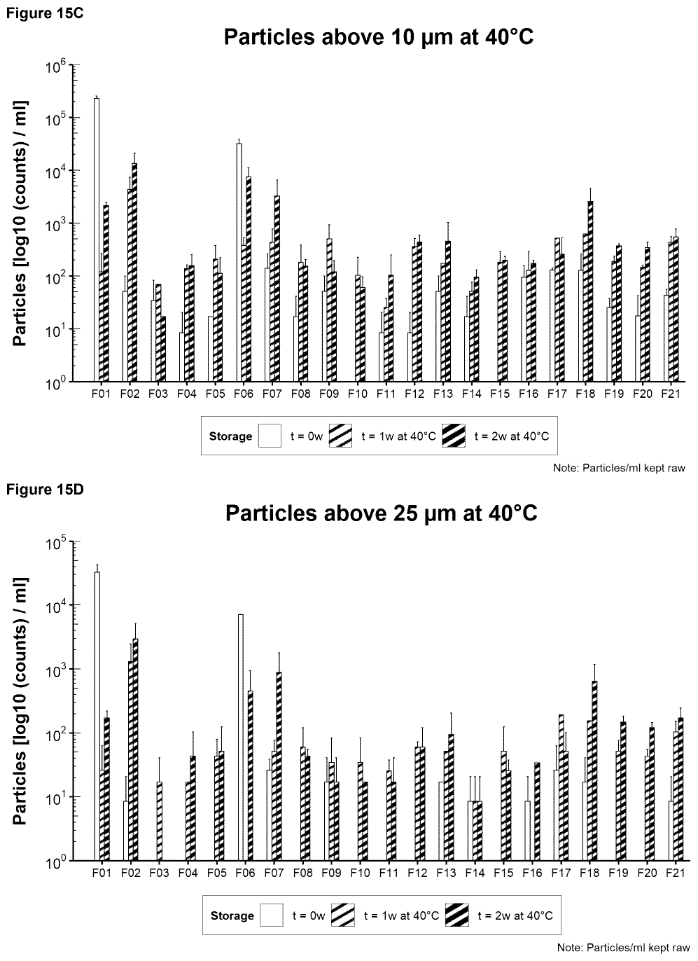

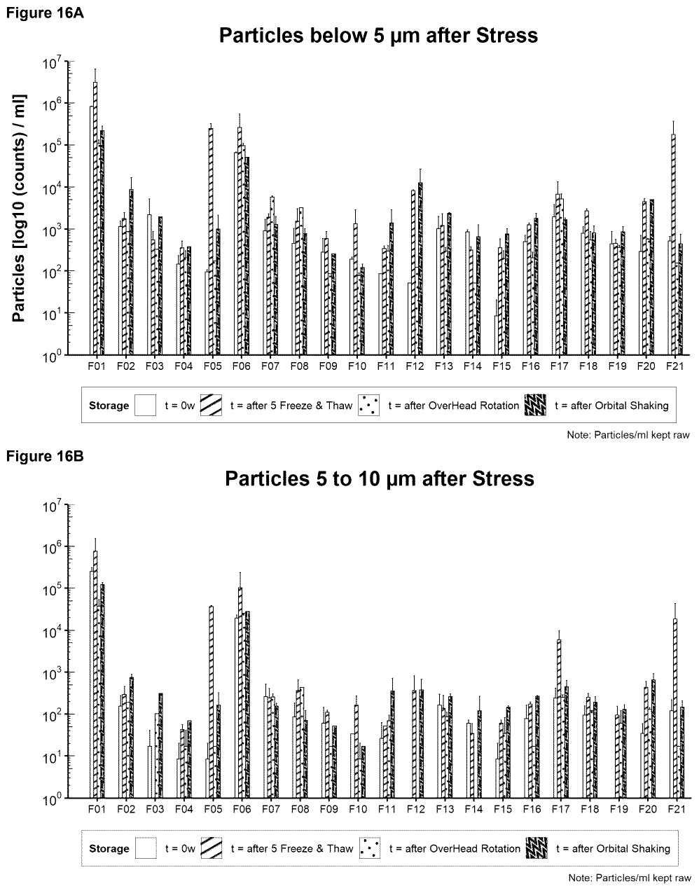

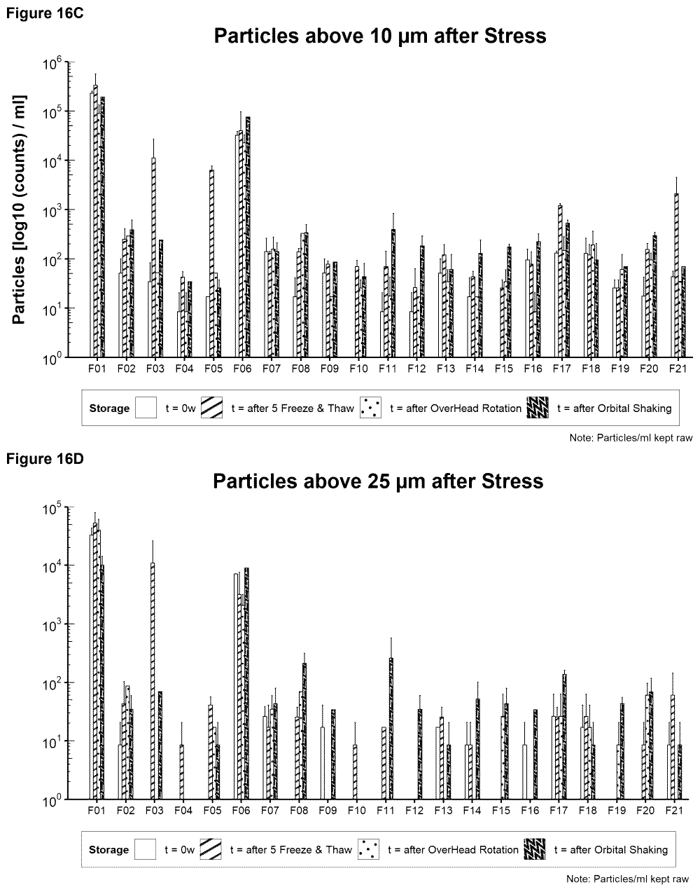

Here, amount of Subvisible particles of samples was determined and the results are shown in FIGS. 15 and 16. In detail, particle content below 5 μm, from 5 to 10 μm, above or equal to 10 μm and above or equal to 25 μm after 2 weeks of storage at 40° C. or after mechanical stress was assessed.

### 5. Visual Appearance

Also, the visual appearance after mechanical stress, Freeze and Thaw and 2 weeks storage at 40° C. was assessed. The results are depicted in Table 8.

### 6. Summary

The main focus of this Formulation Development was the prevention of visible particles. In the screening DoE stressed Formulations 4,10,11,16,18.19 and 20 were exempt of visible particles. Most of these formulations also exhibited low subvisible particle counts. In SE-HPLC and DLS analysis, no distinct effect was observed. However, Formulation 6 and the reference formulation 21 show a distinct higher HMWS content after 5 Freeze and Thaw cycles. Both formulations have no Arginine and no Poloxamer in combination with a high Glycine content. Based on the observed results the following potential effects were further addressed in Example 4.

### Example 4: Design, Test and Optimization of Formulations to Stabilize an Anti-ADM Antibody (HAM8101) and Reduce Particle Formation in Liquid Formulation During Two Rounds of Accelerated Aging-Screening DoE Design-Optimization Round

Based on the results of Example 3, 10 formulations were selected additional to the current formulation (Fit) and subjected to a second round of forced degradation. Stress conditions for Example 4 were identical to those used for Example 3. The composition of the different formulations is shown in Table 9.

### 1. Osmolality and Concentration

First, the osmolality and concentration of samples in Example 4 are described. The measured data are shown in Table 10.

### 2. Size Exclusion-HPLC

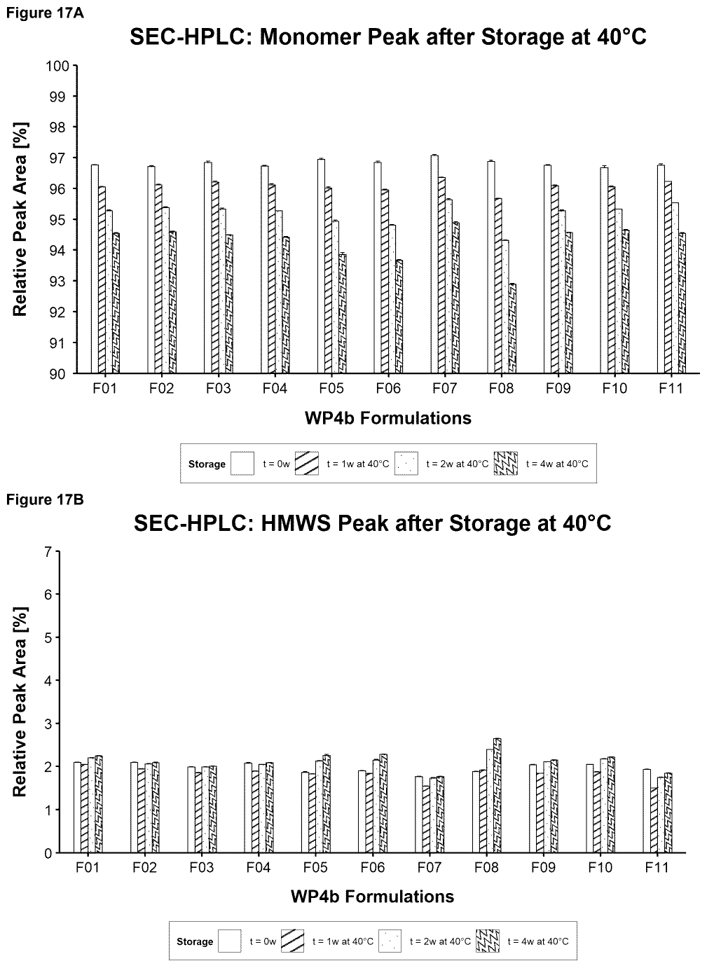

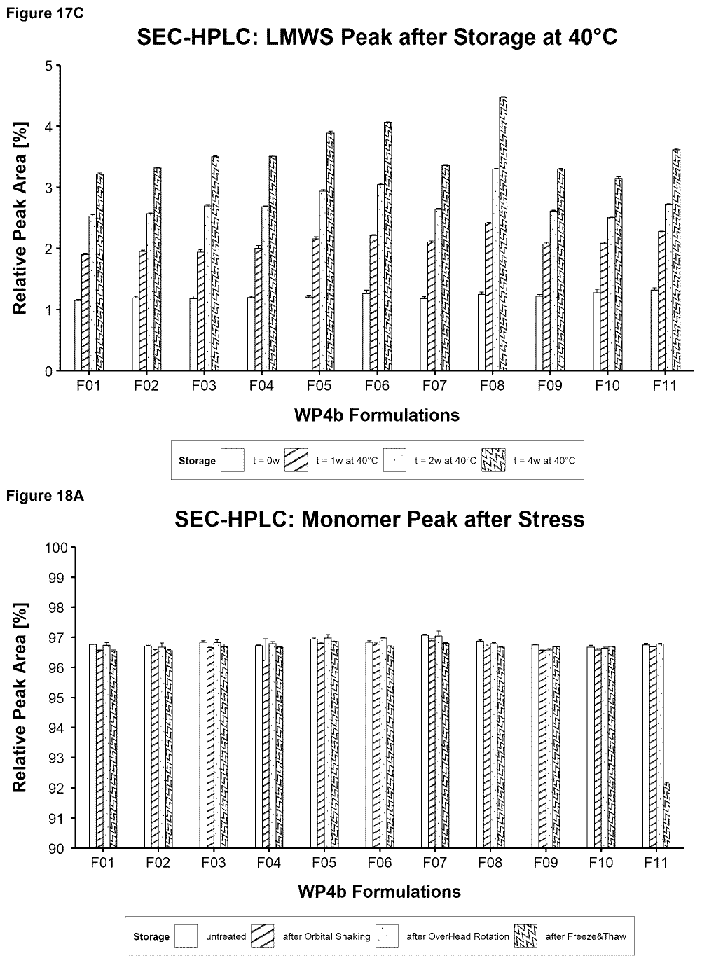

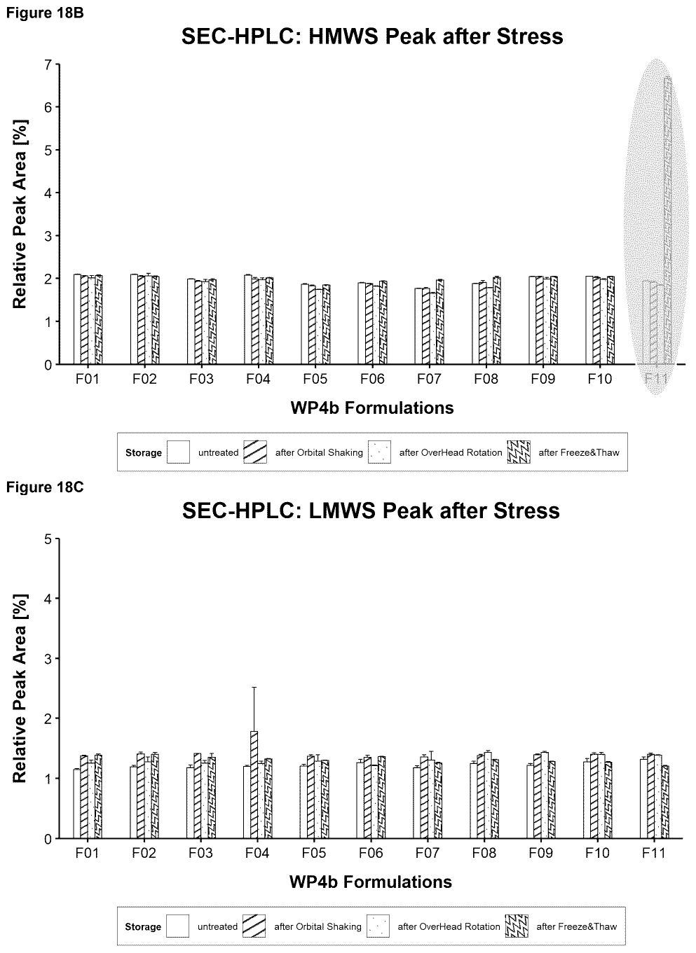

Results of SEC-HPLC were expressed as mean relative peak area [%], derived from two replicates measured in duplicates and displayed in FIGS. 17 and 18. Overall a pronounced fragmentation is detected after 4 weeks at 40° C. and an effect of rising HMW levels in Formulation 11 after 5 Freeze and Thaw cycles.

### 3. AUC of Preselected Samples (1,2,3,4,10,11)

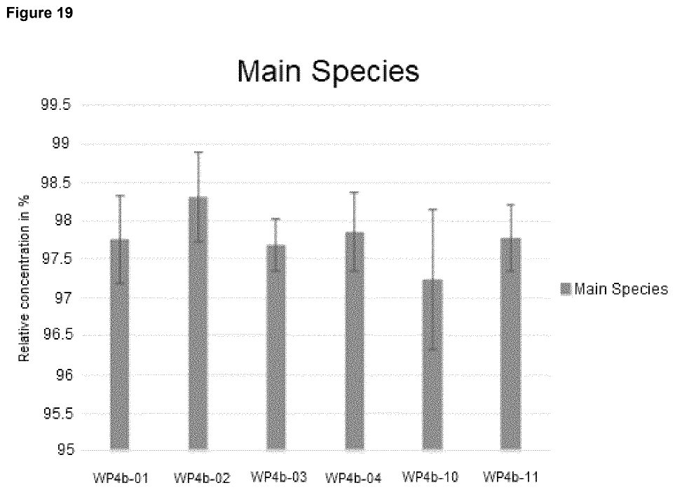

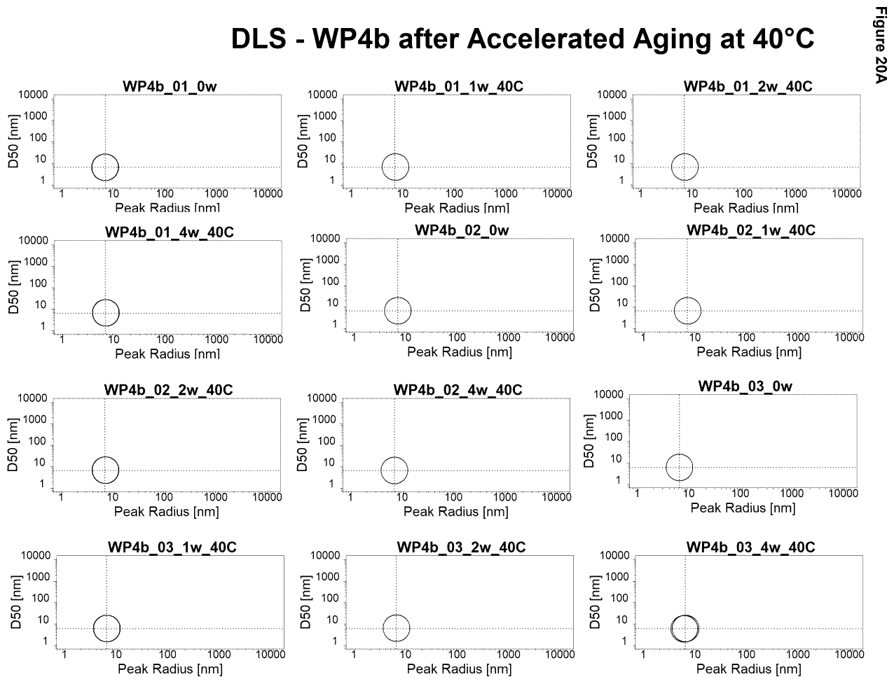

The AUC results of selected samples after 3.5 weeks at 40° C. confirmed the results of SE-HPLC. In total, no distinct aggregation detectable and the formulation WP4b-2 showed the lowest amount (%) of LMWS and HMWS. The results are depicted in Table 11. The relative Monomer content after external analysis via analytical ultracentrifugation is shown in FIG. 19.

### 4. CE-SDS (Red./Non-Red)

Results of CE-SDS non-reduced (Table 12) and reduced (Table 13) were expressed as mean relative peak area derived from two replicates measured in duplicates and displayed in Table 12 and Table 13 respectively. The results of CE-SDS (non-reduced) indicated that no distinct aggregation effect detectable, but a pronounced fragmentation was visible after 3.5 weeks at 40° C. In addition, the lowest fragmentation level could be observed for F01 and F02, whereas the highest fragmentation level was observed for F08 and F11. Notably, the results of CE-SDS (reduced) indicated that no distinct aggregation and fragmentation effect was detectable. In detail, pronounced fragmentation was detectable after 3.5 weeks at 40° C. and the lowest fragmentation level was observed for F02, F03 and F09.

### 5. Dynamic Light Scattering (DLS)

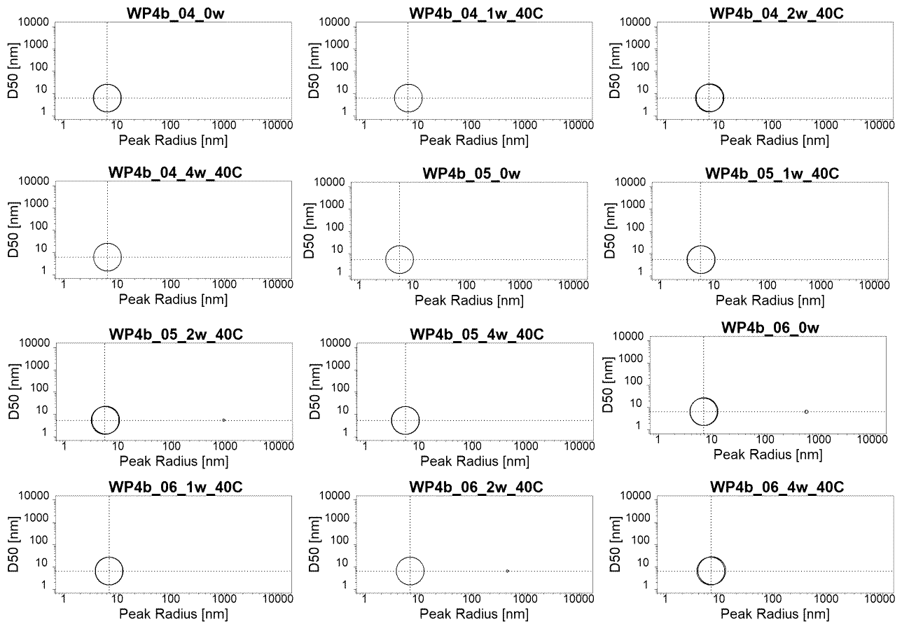

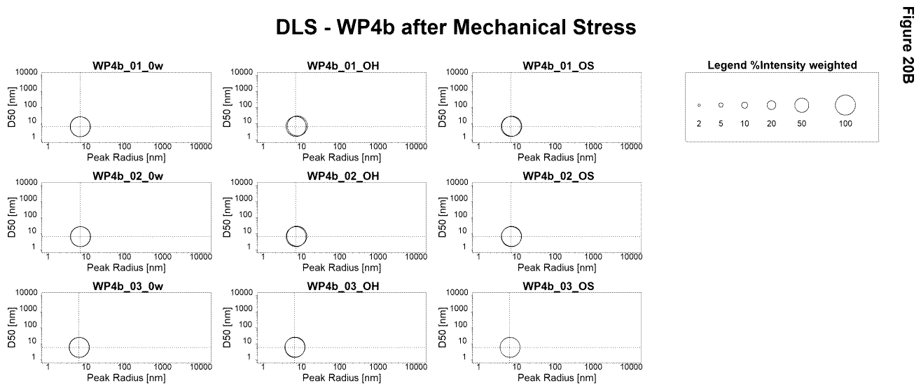

The DLS results after storage at 40° C. and after mechanical stress are depicted in FIG. 20.

### 6. Subvisible Particle Analyses by FlowCam

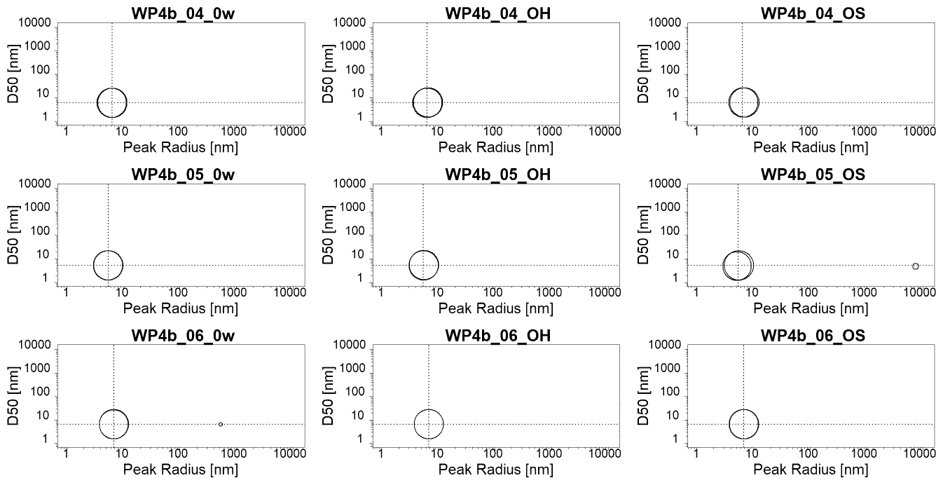

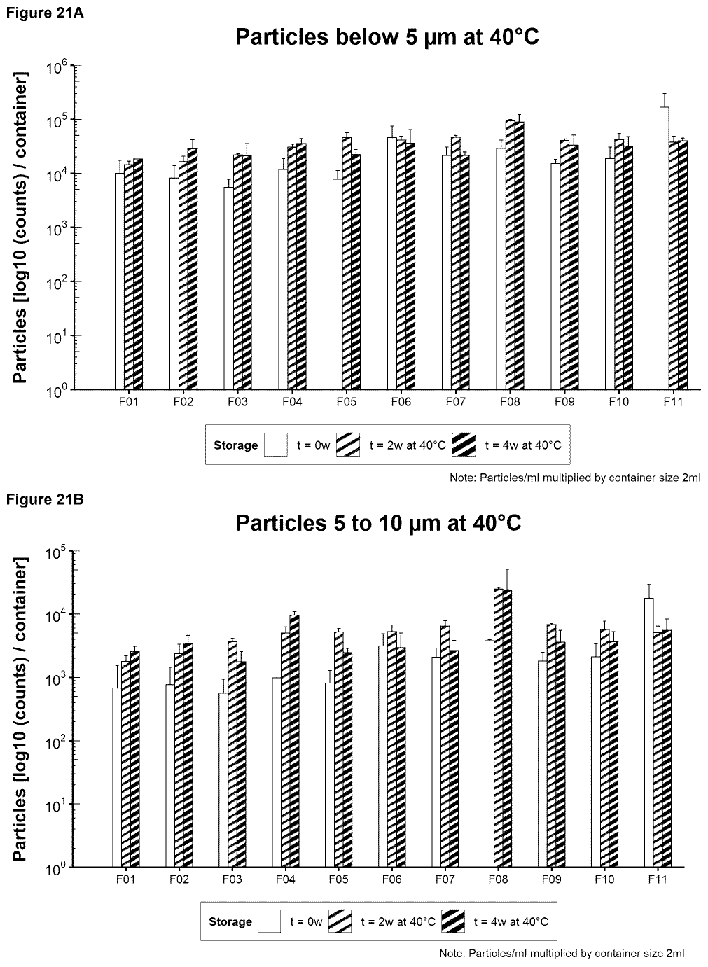

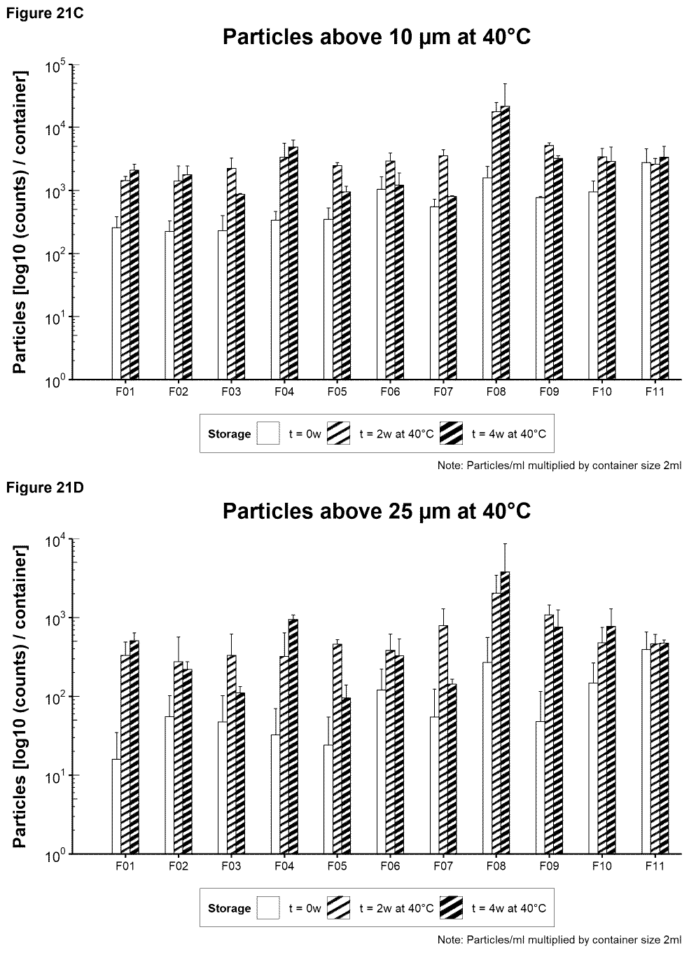

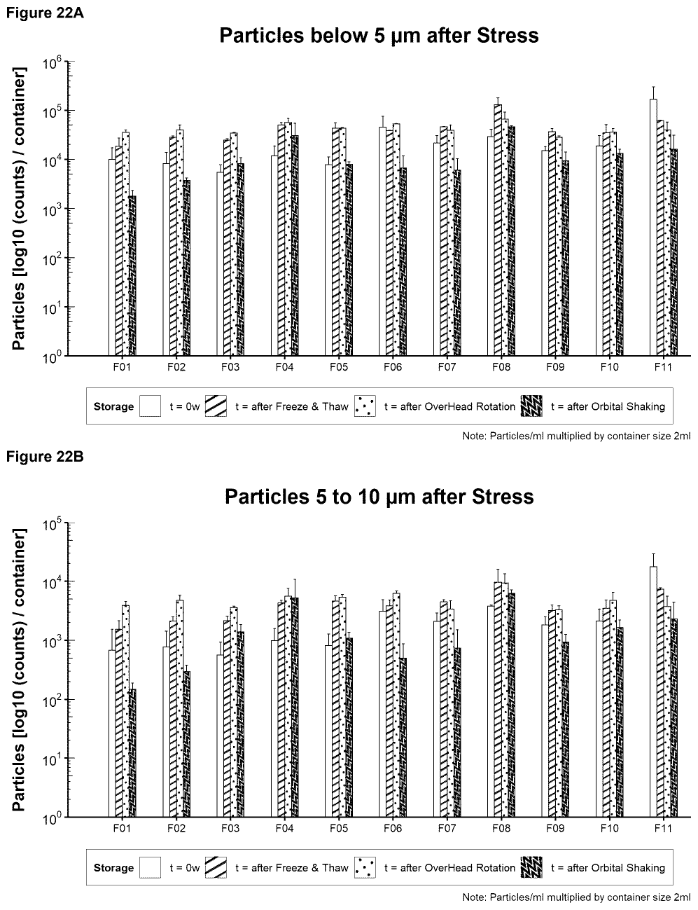

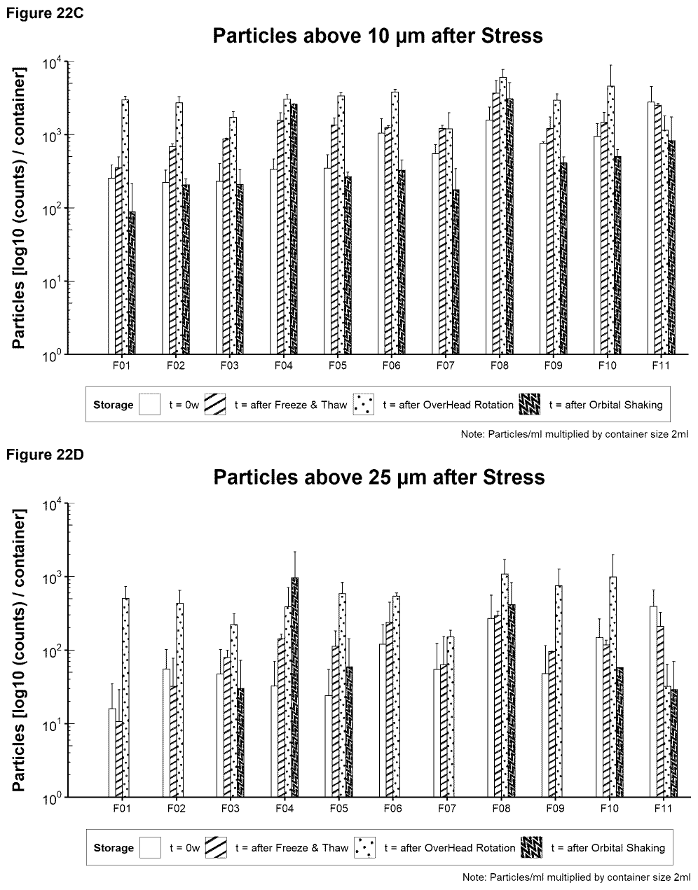

Here, the particles after thermal and mechanical stress were analyzed. The results are shown in FIGS. 21 and 22.

### 7. Visual Appearance

Also, the visual appearance after mechanical stress, Freeze and Thaw and 2 weeks storage at 40° C. was assessed. The results are depicted in Table 14 (Clear: opalescence like water; NVP: no visible particles; turbid: too many particles to count).

### 8. Cation Exchange HPLC

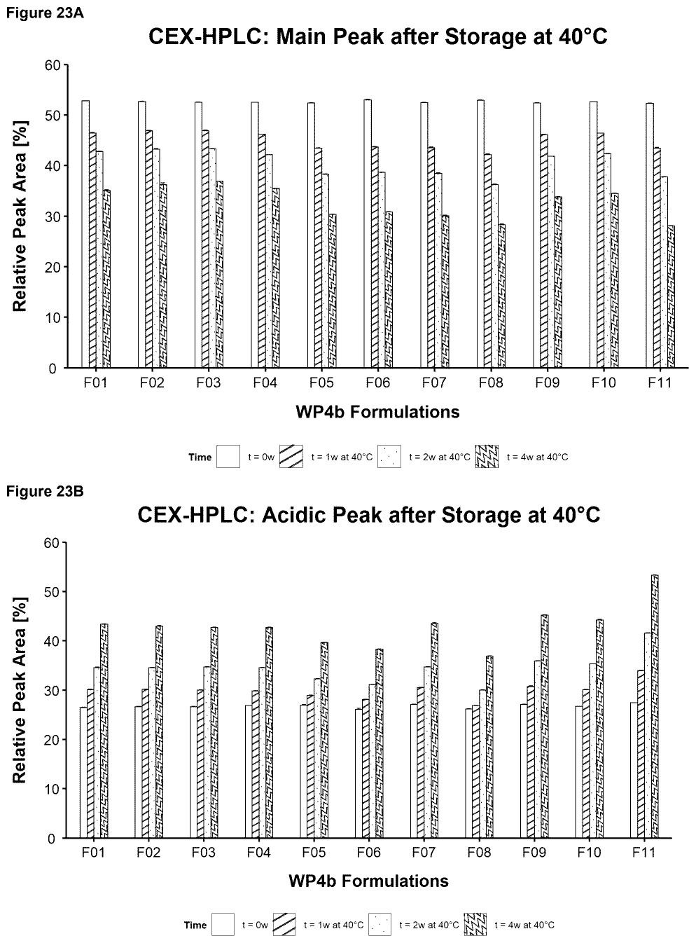

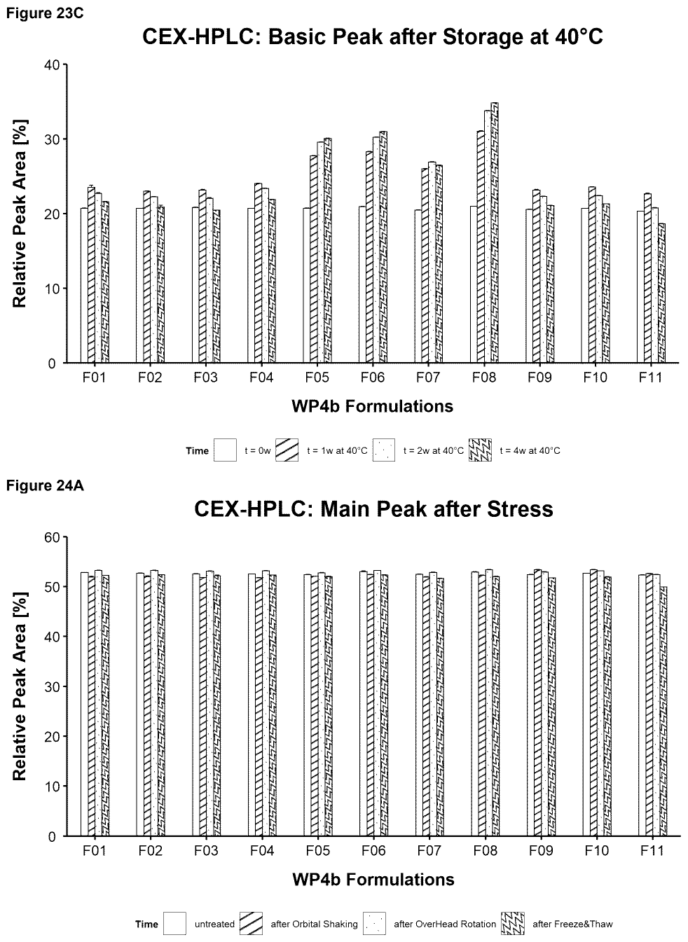

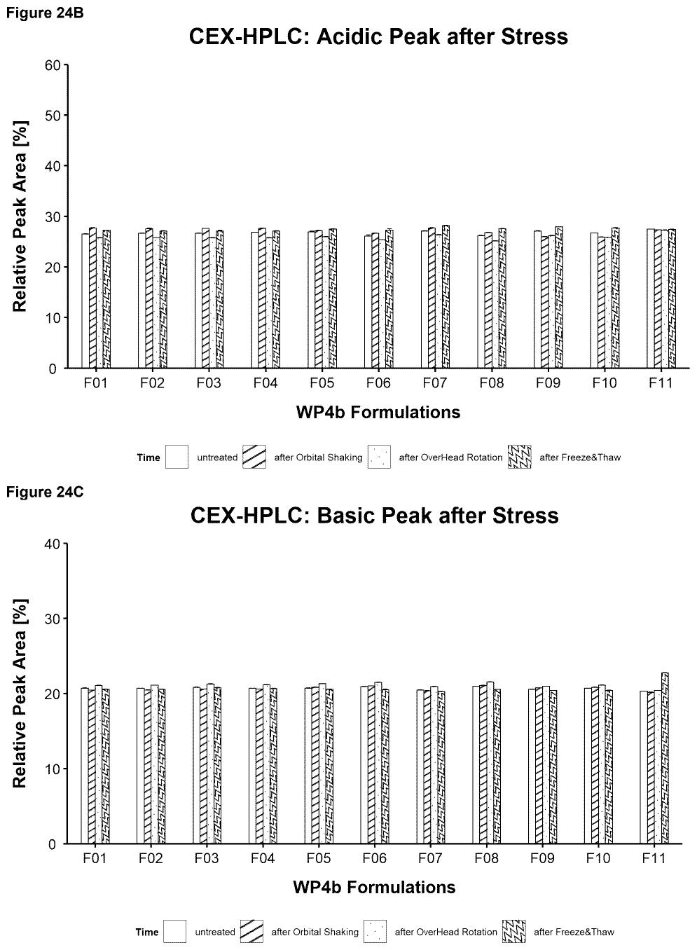

Relative area [%] of Main (above), acidic (middle) and basic (below) species of CEX-HPLC of formulated HAM8101 stored for up to 3.5 weeks at 40° C.±2° C./75% rH±5% rH or after mechanical stress and 5 Freeze & Thaw cycles was assessed. The results are shown in FIGS. 23 and 24.

TABLE 1
Gradient for CEX analysis

| Time [min] | Eluent A [%] | Eluent B [%] |
|---|---|---|
| 0 | 100 | 0 |
| 5 | 100 | 0 |
| 10 | 70 | 30 |
| 25 | 35 | 65 |
| 27 | 0 | 100 |
| 32 | 0 | 100 |
| 35 | 100 | 0 |
| 40 | 100 | 0 |

TABLE 2
Storage conditions

| | Time Point in Weeks | | | | |
|---|---|---|---|---|---|
| Stress Type | t = 0 | t = 3 days | t = 1 week | t = 2 weeks | t = 3 weeks |
| 5° C. ± 3° C. | X | X | X | X | X |
| 25° C. ± 2° C./60% RH ± 5% RH | | X | X | X | X |
| 30° C. ± 2° C./65% RH ± 5% RH | | X | X | X | X |
| 40° C. ± 2° C./75% RH ± 5% RH | | X | X | X | X |

| | |
|---|---|
| Mechanical stressing (up to 24 h) | Orbital shaking and overhead rotation |
| Freeze/thaw (−70° C./+25° C.) | after 5 cycles |

TABLE 3
Visual appearance (color, clarity and visible particles)
after stress and subsequent freezing at −80° C.
Color and Visual appearance after stress and
subsequent freezing at −80° C.

| Sample name | Color and clarity | Visible particles |
|---|---|---|
| WP3_01_0d | clear | NVP |
| WP3_01_3d_5C | clear | NVP |
| WP3_01_3d_25C | clear | NVP |
| WP3_01_3d_30C | clear | NVP |
| WP3_01_3d_40C | clear | few white particles |
| WP3_01_7d_5C | clear | NVP |
| WP3_01_7d_25C | clear | NVP |
| WP3_01_7d_30C | clear | NVP |
| WP3_01_7d_40C | clear | few white particles |
| WP3_01_14d_5C | clear | NVP |
| WP3_01_14d_25C | clear | NVP |
| WP3_01_14d_30C | clear | NVP |
| WP3_01_14d_40C | clear | NVP |
| WP3_01_21d_5C | clear | NVP |
| WP3_01_21d_25C | clear | few white particles |
| WP3_01_21d_30C | clear | few white particles |
| WP3_01_21d_40C | clear | few white particles |
| WP3_01_FT | clear | few white particles |
| WP3_01_OH | clear | few white particles |
| WP3_01_OS | clear | few white particles |

TABLE 4
Visual appearance (color, clarity and visible particles) after stress
and subsequent freezing at −80° C. and aliquotation for analytics
and further cycle of freezing at −80° C.
Color and Visual inspection after stress and subsequent freezing at −80°
C. + aliquotation for analytics and further cycle of freezing at −80° C.

| Sample name | Color and clarity | Appearance |
|---|---|---|
| WP3_01_0d | clear | NVP |
| WP3_01_3d_5C | clear | few white particles |
| WP3_01_3d_25C | clear | few white particles |
| WP3_01_3d_30C | clear | few white particles |
| WP3_01_3d_40C | clear | many white particles |
| WP3_01_7d_5C | clear | few white particles |
| WP3_01_7d_25C | clear | few white particles |
| WP3_01_7d_30C | clear | many white particles |
| WP3_01_7d_40C | clear | many white particles |
| WP3_01_14d_5C | clear | few white particles |
| WP3_01_14d_25C | clear | few white particles |
| WP3_01_14d_30C | clear | few white particles |
| WP3_01_14d_40C | clear | few white particles |
| WP3_01_21d_5C | clear | few white particles |
| WP3_01_21d_25C | clear | few white particles |
| WP3_01_21d_30C | clear | many white particles |
| WP3_01_21d_40C | clear | many white particles |
| WP3_01_FT | clear | many white particles |
| WP3_01_OH | clear | many white particles |
| WP3_01_OS | clear | many white particles |

TABLE 5
DoE excipient concentration

| Factor | min | max | | rationale |
|---|---|---|---|---|
| His/HCL | 10 mM | 10 | mM | Fixed Buffer system and antioxidative efficacy |
| pH | 5 | 7 | | balance between colloidal stability and theo. |
| | | | | deamidation and or isomerization |
| Glycine | 0 | 150 | mM | osmolytic agent control of polypeptide flexibility |
| Trehalose | 0 | 150 | mM | cryoprotectant, osmolytic agent |
| NaCl | 0 | 150 | mM | balanced ionic strength and osmolytic agent |
| Poloxamer 188 | 0 | 0.02% | | Surfactant |
| EDTA | 0 | 5 | mM | Chelating agent |
| Arginine | 0 | 150 | mM | Colloidal stabilizer |

TABLE 6
Composition of DoE based formulations
g/L

| Run | Arginine | pH | Trehalose | NaCl | Glycine | NA. EDTA | Poloxamer_188 | Histidine | Block |
|---|---|---|---|---|---|---|---|---|---|
| WP4a_01 | 0.0 | 7.0 | 56.7 | 0.0 | 0.0 | 1.9 | 0.5 | 1.6 | cube |
| WP4a_02 | 26.1 | 5.0 | 0.0 | 8.8 | 11.3 | 0.0 | 0.0 | 1.6 | cube |
| WP4a_03 | 0.0 | 5.0 | 56.7 | 0.0 | 11.3 | 0.0 | 0.5 | 1.6 | cube |
| WP4a_04 | 26.1 | 5.0 | 56.7 | 8.8 | 0.0 | 0.0 | 0.5 | 1.6 | cube |
| WP4a_05 | 0.0 | 5.0 | 0.0 | 0.0 | 0.0 | 0.0 | 0.0 | 1.6 | cube |
| WP4a_06 | 0.0 | 7.0 | 0.0 | 0.0 | 11.3 | 1.9 | 0.0 | 1.6 | cube |
| WP4a_07 | 0.0 | 5.0 | 56.7 | 8.8 | 11.3 | 1.9 | 0.0 | 1.6 | cube |
| WP4a_08 | 0.0 | 7.0 | 0.0 | 8.8 | 11.3 | 0.0 | 0.5 | 1.6 | cube |
| WP4a_09 | 13.1 | 6.0 | 28.4 | 4.4 | 5.6 | 0.9 | 0.3 | 1.6 | cube_center1 |
| WP4a_10 | 26.1 | 7.0 | 0.0 | 0.0 | 0.0 | 0.0 | 0.5 | 1.6 | cube |
| WP4a_11 | 26.1 | 7.0 | 56.7 | 8.8 | 11.3 | 1.9 | 0.5 | 1.6 | cube |
| WP4a_12 | 26.1 | 7.0 | 56.7 | 0.0 | 11.3 | 0.0 | 0.0 | 1.6 | cube |
| WP4a_13 | 26.1 | 7.0 | 0.0 | 8.8 | 0.0 | 1.9 | 0.0 | 1.6 | cube |
| WP4a_14 | 26.1 | 5.0 | 0.0 | 0.0 | 11.3 | 1.9 | 0.5 | 1.6 | cube |
| WP4a_15 | 13.1 | 6.0 | 28.4 | 4.4 | 5.6 | 0.9 | 0.3 | 1.6 | cube_center2 |
| WP4a_16 | 0.0 | 7.0 | 0.0 | 8.8 | 0.0 | 1.9 | 0.5 | 1.6 | Cube |
| WP4a_17 | 0.0 | 5.0 | 56.7 | 8.8 | 0.0 | 0.0 | 0.0 | 1.6 | Cube |
| WP4a_18 | 26.1 | 5.0 | 56.7 | 0.0 | 0.0 | 1.9 | 0.0 | 1.6 | Cube |
| WP4a_19 | 13.1 | 6.0 | 28.4 | 4.4 | 5.6 | 0.9 | 0.3 | 1.6 | Cube_center3 |
| WP4a_20 | 13.1 | 6.0 | 28.4 | 4.4 | 5.6 | 0.9 | 0.3 | 1.6 | Cube_center4_N2 |
| WP4a_21 | | 6.0 | 0.0 | 0.0 | 22.5 | 0.0 | 0.0 | 3.1 | reference |

TABLE 7
Measured Osmolality in mOsmol/L (in Vial A, B and Placebo) and
concentration in g/L (mean + SD) after manufacturing and storage

| mOsmol/L | | | | g/L |
|---|---|---|---|---|
| 0 w | A | B | Placebo | concentration |
| WP4a_01_0w | 200 | 209 | 200 | 20 ± 1 |
| WP4a_02_0w | 712 | 725 | 719 | 20 ± 1 |
| WP4a_03_0w | 353 | 347 | 346 | 20 ± 1 |
| WP4a_04_0w | 797 | 783 | 799 | 20 ± 1 |
| WP4a_05_0w | 38 | 30 | 23 | 20 ± 1 |
| WP4a_06_0w | 198 | 193 | 185 | 20 ± 1 |
| WP4a_07_0w | 691 | 703 | 663 | 20 ± 1 |
| WP4a_08_0w | 465 | 476 | 455 | 20 ± 1 |
| WP4a_09_0w | 483 | 469 | 489 | 20 ± 1 |
| WP4a_10_0w | 309 | 298 | 299 | 20 ± 1 |
| WP4a_11_0w | 986 | 1005 | 987 | 20 ± 1 |
| WP4a_12_0w | 654 | 661 | 668 | 20 ± 1 |
| WP4a_13_0w | 616 | 615 | 605 | 20 ± 1 |
| WP4a_14_0w | 507 | 488 | 471 | 20 ± 1 |
| WP4a_15_0w | 489 | 505 | 469 | 20 ± 1 |
| WP4a_16_0w | 349 | 354 | 330 | 20 ± 1 |
| WP4a_17_0w | 528 | 518 | 523 | 20 ± 1 |
| WP4a_18_0w | 526 | 516 | 526 | 20 ± 1 |
| WP4a_19_0w | 516 | 520 | 497 | 20 ± 1 |
| WP4a_20_0w | 517 | 524 | 521 | 20 ± 1 |
| WP4a_21_0w | 365 | 364 | 385 | 20 ± 1 |

TABLE 8
Visual appearance after mechanical stress, Freeze and Thaw and 2 weeks storage at 40° C.

| | 0 w | Overhead rotation | Orbital shaking | Freeze/thaw* | 1 w_40 C. | 2 w_40 C. |
|---|---|---|---|---|---|---|
| | A and B Vial | A and B Vial | A and B Vial | A and B Vial | A and B Vial | A and B Vial |
| F_1 | slightly turbid, | Slightly turbid, few | clear, white | Slightly turbid, NVP | slightly turbid, 1 | slightly turbid, |
| | NVP | white particles | particle | | white particle | NVP |
| F_2 | clear, NVP | clear, 1 white particle | clear, NVP | clear, many | clear, NVP | clear, few |
| | | | | particles | | white particles |
| F_3 | clear, 2 white | clear, NVP | clear, NVP | clear, NVP | clear, NVP | clear, NVP |
| | particle | | | | | |
| F_4 | clear, NVP | clear, NVP | clear, NVP | clear, NVP | clear, NVP | clear, NVP |
| F_5 | clear, 1 white | clear, NVP | clear, NVP | clear, many | clear, NVP | clear, NVP |
| | particle | | | particles | | |
| F_6 | slightly turbid, | turbid, white particle | Clear, many | very turbid, many | slightly turbid | slightly turbid, |
| | NVP | | white particles | particles, strong air | | NVP |
| | | | | bubble formation | | |
| F_7 | clear, NVP | clear, NVP | clear, NVP | clear, few white | clear, NVP | clear, NVP |
| | | | | particles | | |
| F_8 | clear, NVP | clear, NVP | clear, NVP | clear, 2 white | clear, NVP | clear, NVP |
| | | | | particles | | |
| F_9 | clear, NVP | clear, NVP | clear, 1 white | clear, 2 white | clear, few white | clear, NVP |
| | | | particles | particles | particle | |
| F_10 | clear, NVP | clear, NVP | clear, NVP | clear, NVP | clear, NVP | clear, NVP |
| F_11 | clear, NVP | clear, NVP | clear, NVP | clear, NVP | clear, NVP | clear, NVP |
| F_12 | clear, NVP | clear, NVP | clear, 2 white | clear, few white | clear, NVP | clear, NVP |
| | | | particle | particles | | |
| F_13 | clear, NVP | clear, NVP | clear, NVP | clear, few white | clear, 1 white | clear, NVP |
| | | | | particles, strong air | particle | |
| | | | | bubble formation | | |
| F_14 | Clear, 1 white | clear, NVP | clear, NVP | clear, NVP | clear, NVP | clear, NVP |
| | particle | | | | | |
| F_15 | clear, NVP | clear, NVP | clear, NVP | clear, 1 white | clear, NVP | clear, NVP |
| | | | | particle | | |
| F_16 | clear, NVP | clear, NVP | clear, NVP | clear, NVP | clear, NVP | clear, NVP |
| F_17 | clear, NVP | clear, NVP | clear, NVP | turbid, many white | clear, NVP | clear, NVP |
| | | | | particles | | |
| F_18 | clear, NVP | clear, NVP | clear, NVP | clear, NVP | clear, NVP | clear, NVP |
| F_19 | clear, NVP | clear, NVP | clear, NVP | clear, NVP | clear, NVP | clear, NVP |
| F_20 | clear, NVP | clear, NVP | clear, NVP | clear, NVP | clear, NVP | clear, NVP |
| F_21 | clear, 2 white | clear, NVP | clear, 1 white | clear, many white | clear, few white | clear, 2 white |
| | particle | | particle | particles | particle | particles |

TABLE 9
Formulation matrix

| | Arginine | | Trehalose | NaCl | Glycine | Poloxamer_188 | Histidine | Lysin |
|---|---|---|---|---|---|---|---|---|
| Run | in g/L | pH | in g/L | in g/L | in g/L | in g/L | in g/L | in g/L |
| WP4b_01 | 13.1 | 6.0 | 56.7 | 4.4 | | 0.5 | 1.6 | |
| WP4b_02 | 26.1 | 6.0 | 56.7 | | | 0.5 | 1.6 | |
| WP4b_03 | 52.2 | 6.0 | | | | 0.5 | 1.6 | |
| WP4b_04 | 26.1 | 6.0 | | | | | 1.6 | 20 |
| WP4b_05 | 13.1 | 5.0 | | | | 0.5 | 1.6 | |
| WP4b_06 | 26.1 | 5.0 | 56.7 | | | 0.5 | 1.6 | |
| WP4b_07 | | 5.0 | | | | 0.5 | 1.6 | |
| WP4b_08 | | 5.0 | | | | | 1.6 | 45 |
| WP4b_09 | 13.1 | 6.0 | 28.4 | 4.4 | 5.6 | 0.25 | 1.6 | |
| WP4b_10 | 13.1 | 6.0 | 28.4 | 4.4 | | 0.25 | 1.6 | |
| WP4b_11 | | 6.0 | | | 22.5 | | 3.2 | |

TABLE 10
Measured osmolality in mOsmol/L (in vial A, B) and concentration
in g/L (mean + SD) after manufacturing and storage

| mOsmol/L | | | | g/L |
|---|---|---|---|---|
| | A | B | Mean | concentration |
| WP4a_01_0w | 476 | 479 | 478 | 20 ± 1 |
| WP4a_02_0w | 471 | 475 | 473 | 20 ± 1 |
| WP4a_03_0w | 528 | 535 | 532 | 20 ± 1 |
| WP4a_04_0w | 544 | 556 | 550 | 20 ± 1 |
| WP4a_05_0w | 163 | 156 | 160 | 20 ± 1 |
| WP4a_06_0w | 458 | 467 | 463 | 20 ± 1 |
| WP4a_07_0w | 21 | 21 | 21 | 20 ± 1 |
| WP4a_08_0w | 563 | 564 | 564 | 20 ± 1 |
| WP4a_09_0w | 456 | 450 | 453 | 20 ± 1 |
| WP4a_10_0w | 385 | 374 | 380 | 20 ± 1 |
| WP4a_11_0w | 328 | 327 | 328 | 20 ± 1 |

TABLE 11
Results of the analytical ultracentrifugation

| | | Absorbance Optics 280 nm | | |
|---|---|---|---|---|
| Sample | Species | s(S) | Mw (kDa) | Amount (%) |
| WP4b-1 | LMWS | 1.96 ± 0.19 | 28 ± 4 | 0.7 ± 0.1 |
| | Main Species | 5.99 ± 0.00 | 146 ± 0 | 97.7 ± 0.6 |
| | HMWS 1 | 9.45 ± 0.09 | 289 ± 4 | 1.4 ± 0.4 |
| | HMWS 2 | 12.03 ± 0.27 | 418 ± 15 | 0.2 ± 0.3 |
| WP4b-2 | LMWS | 2.12 ± 0.20 | 32 ± 4 | 0.5 ± 0.1 |
| | Main Species | 5.80 ± 0.00 | 145 ± 0 | 98.3 ± 0.6 |
| | HMWS 1 | 9.30 ± 0.14 | 294 ± 6 | 1.1 ± 0.3 |
| | HMWS 2 | 13.67 ± 1.38 | 531 ± 83 | 0.1 ± 0.2 |
| WP4b-3 | LMWS | 2.01 ± 0.21 | 33 ± 5 | 0.6 ± 0.0 |
| | Main Species | 5.45 ± 0.00 | 143 ± 0 | 97.7 ± 0.3 |
| | HMWS 1 | 8.64 ± 0.10 | 285 ± 5 | 1.5 ± 0.1 |
| | HMWS 2 | 11.56 ± 1.85 | 447 ± 112 | 0.2 ± 0.2 |
| WP4b-4 | LMWS | 2.31 ± 0.24 | 39 ± 6 | 0.6 ± 0.1 |
| | Main Species | 5.52 ± 0.00 | 143 ± 0 | 97.8 ± 0.5 |
| | HMWS 1 | 8.78 ± 0.14 | 286 ± 7 | 1.4 ± 0.3 |
| | HMWS 2 | 11.45 ± 0.61 | 428 ± 36 | 0.2 ± 0.2 |
| WP4b-10 | LMWS | 2.66 ± 0.15 | 42 ± 4 | 0.5 ± 0.1 |
| | Main Species | 6.09 ± 0.00 | 146 ± 0 | 97.2 ± 0.9 |
| | HMWS 1 | 9.49 ± 0.18 | 285 ± 8 | 1.7 ± 0.5 |
| | HMWS 2 | 12.97 ± 0.14 | 455 ± 6 | 0.6 ± 0.5 |
| WP4b-11 | LMWS | 2.97 ± 0.13 | 50 ± 3 | 0.9 ± 0.1 |
| | Main Species | 6.20 ± 0.00 | 147 ± 0 | 97.8 ± 0.4 |
| | HMWS 1 | 9.70 ± 0.14 | 288 ± 6 | 1.1 ± 0.3 |
| | HMWS 2 | 14.15 ± 0.51 | 509 ± 29 | 0.3 ± 0.2 |

TABLE 12
Relative results of the non-reduced CE SDS analysis

| Main Peak | | | | | HMWS | | | | LMWS | | | |
|---|---|---|---|---|---|---|---|---|---|---|---|---|
| | | | | T4 | | | | T4 | | | | T4 |
| | T0 | T1 | T2 | (3.5) | T0 | T1 | T2 | (3.5) | T0 | T1 | T2 | (3.5) |
| | mean | mean | mean | mean | mean | mean | mean | mean | mean | mean | mean | mean |
| | (A/B) | (A/B) | (A/B) | (A/B) | (A/B) | (A/B) | (A/B) | (A/B) | (A/B) | (A/B) | (A/B) | (A/B) |
| F01 | 96% | 91% | 88% | 86% | 0.3% | 0.7% | 0.8% | 0.8% | 4.2% | 8.5% | 11.5% | 13.1% |
| F02 | 96% | 91% | 88% | 87% | 0.4% | 0.7% | 0.7% | 0.7% | 4.1% | 8.5% | 11.0% | 12.7% |
| F03 | 96% | 90% | 87% | 85% | 0.2% | 0.4% | 0.7% | 0.6% | 4.2% | 9.4% | 12.5% | 14.5% |
| F04 | 94% | 90% | 88% | 83% | 1.5% | 0.5% | 0.9% | 0.9% | 4.1% | 9.9% | 10.9% | 16.0% |
| F05 | 95% | 90% | 85% | 82% | 0.3% | 0.6% | 0.7% | 0.8% | 4.2% | 9.5% | 14.0% | 17.5% |
| F06 | 95% | 89% | 86% | 82% | 0.5% | 0.4% | 0.6% | 0.8% | 4.3% | 10.9% | 13.0% | 17.4% |
| F07 | 95% | 89% | 86% | 82% | 0.4% | 0.4% | 0.4% | 0.6% | 4.4% | 10.6% | 13.8% | 17.8% |
| F08 | 95% | 88% | 84% | 79% | 0.4% | 0.6% | 0.6% | 0.5% | 4.4% | 11.9% | 15.2% | 20.8% |
| F09 | 95% | 90% | 86% | 84% | 0.4% | 0.7% | 0.6% | 0.7% | 4.3% | 9.6% | 12.9% | 14.9% |
| F10 | 95% | 90% | 87% | 85% | 0.7% | 0.8% | 0.7% | 0.8% | 4.3% | 9.1% | 12.2% | 14.1% |
| F11 | 95% | 89% | 87% | 80% | 0.4% | 0.5% | 0.4% | 0.8% | 4.4% | 10.1% | 13.1% | 19.2% |

TABLE 13
Relative results of the reduced CE SDS analysis

| HC | | | | | LC | | | | HMWS | | | | LMWS | | | |
|---|---|---|---|---|---|---|---|---|---|---|---|---|---|---|---|---|
| | | | | T4 | | | | T4 | | | | T4 | | | | T4 |
| | T0 | T1 | T2 | (3.5) | T0 | T1 | T2 | (3.5) | T0 | T1 | T2 | (3.5) | T0 | T1 | T2 | (3.5 |
| | mean | mean | mean | mean | mean | mean | mean | mean | mean | mean | mean | mean | mean | mean | mean | mean |
| | (A/B) | (A/B) | (A/B) | (A/B) | (A/B) | (A/B) | (A/B) | (A/B) | (A/B) | (A/B) | (A/B) | (A/B) | (A/B) | (A/B) | (A/B) | (A/B) |
| F01 | 69.5 | 68.1 | 67.1 | 65.1 | 27.6 | 27.5 | 27.5 | 27.5 | 0.9 | 0.9 | 1.0 | 1.2 | 2.0 | 3.5 | 4.3 | 6.3 |
| F02 | 69.5 | 68.0 | 66.9 | 62.2 | 27.6 | 27.5 | 27.6 | 27.4 | 0.9 | 0.9 | 1.2 | 4.4 | 2.0 | 3.6 | 4.2 | 6.1 |
| F03 | 67.8 | 67.9 | 67.2 | 65.5 | 27.9 | 27.5 | 27.9 | 27.6 | 2.4 | 2.4 | 1.1 | 1.0 | 2.0 | 3.6 | 3.8 | 5.9 |
| F04 | 68.1 | 66.7 | 67.2 | 64.7 | 27.6 | 27.5 | 27.8 | 27.6 | 2.3 | 2.3 | 0.9 | 1.0 | 2.0 | 3.7 | 4.1 | 6.7 |
| F05 | 69.2 | 67.5 | 65.9 | 63.5 | 27.4 | 27.5 | 27.5 | 27.6 | 1.2 | 1.2 | 0.9 | 1.0 | 2.1 | 4.2 | 5.7 | 7.9 |
| F06 | 67.9 | 67.4 | 66.2 | 62.8 | 27.6 | 27.5 | 27.9 | 27.4 | 2.4 | 2.4 | 0.8 | 1.8 | 2.1 | 4.1 | 5.1 | 7.9 |
| F07 | 69.1 | 67.7 | 66.2 | 63.8 | 27.4 | 27.4 | 27.4 | 27.4 | 1.4 | 1.4 | 1.0 | 1.0 | 2.1 | 4.0 | 5.4 | 7.8 |
| F08 | 68.8 | 67.2 | 65.3 | 64.0 | 27.5 | 27.5 | 27.4 | 27.6 | 1.7 | 1.7 | 1.0 | 0.9 | 2.0 | 4.6 | 6.3 | 7.5 |
| F09 | 68.3 | 68.0 | 66.9 | 65.2 | 27.5 | 27.4 | 27.5 | 27.6 | 2.1 | 2.1 | 1.1 | 1.1 | 2.1 | 3.6 | 4.6 | 6.1 |
| F10 | 68.7 | 68.2 | 67.0 | 64.8 | 27.6 | 27.4 | 27.5 | 27.5 | 1.6 | 1.6 | 1.1 | 1.3 | 2.1 | 3.3 | 4.5 | 6.4 |
| F11 | 67.7 | 67.3 | 65.9 | 63.2 | 27.4 | 27.5 | 27.4 | 27.7 | 2.9 | 2.9 | 1.0 | 1.0 | 2.1 | 4.3 | 5.7 | 8.1 |

TABLE 14
Visual appearance

| | | Overhead | Orbital | Freeze/ | | | |
|---|---|---|---|---|---|---|---|
| | 0 w | rotation | shaking | thaw* | 1 w 40 C. | 2 w 40 C. | 4 w 40 C. |
| | A and B Vial | A and B Vial | A and B Vial | A and B Vial | A and B Vial | A and B Vial | A and B Vial |
| F_1 | clear, 2 white | clear, 1 white | clear, NVP | Y7, NVP | clear, NVP | clear, NVP | Y7, NVP |
| | particles | particle, air | | | | | |
| | | bubble formation | | | | | |
| F_2 | clear, NVP | clear, NVP | clear, NVP | Y6, 1 white | clear, NVP | A: clear, 1 | Y6, NVP |
| | | | | particle | | white fibre | |
| F_3 | clear, NVP | clear, NVP | clear, NVP | Y6, NVP | clear, NVP | A: clear, 1 | Y7, NVP |
| | | | | | | white | |
| | | | | | | particle | |
| F_4 | clear, NVP | clear, NVP | clear, NVP | Y6, NVP | clear, NVP | Clear, few | Y7, NVP |
| | | | | | | white | |
| | | | | | | particles | |
| F_5 | clear, 2 white | clear, NVP | clear, NVP | Y7, NVP | clear, NVP | clear, NVP | Y7, NVP |
| | particles | | | | | | |
| F_6 | clear, NVP | clear, NVP | clear, NVP | Y7, NVP | clear, 1 white | clear, few | Y7, NVP |
| | | | | | fibre | white | |
| | | | | | | particles | |
| F_7 | clear, NVP | clear, NVP | clear, NVP | Y7, NVP | clear, 2 white | clear, NVP | Y7, few |
| | | | | | particles | | white |
| | | | | | | | particles |
| F_8 | clear, few | A: clear, few | clear, NVP | Y7, NVP | clear, NVP | clear, 1 | Y7, NVP |
| | white particles | white particles | | | | white | |
| | | | | | | package | |
| F_9 | clear, NVP | Clear, 1 white | clear, NVP | Y7, NVP | A: clear, NVP | clear, NVP | Y7, NVP |
| | | fibre, 2 white | | | | | |
| | | particles | | | | | |
| F_10 | clear, 1 white | clear, NVP | clear, 1 white | Y7, 1 white | clear, NVP | clear, NVP | Y7, NVP |
| | particle | | particle, , air | particle | | | |
| | | | bubble | | | | |
| | | | formation | | | | |
| F_11 | clear, 2 white | clear, NVP | clear, NVP | Y7, NVP | clear, few | clear, NVP | Y7, few |
| | particle | | | | white | | white |
| | | | | | particles | | particles |

1. Pharmaceutical aqueous formulation comprising an human or humanized anti-adrenomedullin (ADM) antibody or an anti-adrenomedullin antibody fragment wherein said antibody or fragment binds to the N-terminal part (aa 1-21) of ADM: YRQSMNNFQGLRSFGCRFGTC (SEQ ID No. 1), wherein said antibody or antibody fragment is present in a concentration of 1 mg/ml to 100 mg/ml, preferably 2 mg/ml to 50 mg/ml, more preferably 10 mg/ml to 30 mg/ml, more preferably 15 mg/ml to 25 mg/ml and most preferably 20 mg/ml and wherein said pharmaceutical aqueous formulation is further comprising:
Arginine in a range of 1 g/L to 100 g/L, and
Trehalose in a range of 1 g/L to 100 g/L, and
A surfactant selected from polysorbates and poloxamers in a range of 0.01 g/L to 5 g/L, and
Histidine in a range of 0.1 g/L to 6.4 g/L, and
wherein the pH of the formulation is in the range of 4.0 to 8.0.

2. Pharmaceutical aqueous formulation comprising an human or humanized anti-adrenomedullin (ADM) antibody or an anti-adrenomedullin antibody fragment according to claim 1, wherein said antibody or fragment is a human monoclonal antibody or fragment that binds to the N-terminal region (aa 1-21) of ADM (SEQ ID No. 1) or an antibody fragment thereof wherein the heavy chain comprises the sequences:
CDR1:
SEQ ID NO: 2
GYTFSRYW
CDR2:
SEQ ID NO: 3
ILPGSGST
CDR3:
SEQ ID NO: 4
TEGYEYDGFDY
and wherein the light chain comprises the sequences:
CDR1:
SEQ ID NO: 5
QSIVYSNGNTY
CDR2:
SEQ ID NO: 6
RVS
CDR3:
SEQ ID NO: 7
FQGSHIPYT.

3. Pharmaceutical aqueous formulation comprising an human or humanized anti-adrenomedullin (ADM) antibody or an anti-adrenomedullin antibody fragment according to claim 1, comprising an Anti-Adrenomedullin antibody directed to the N-terminal end of Adrenomedullin comprising the following sequence as a heavy chain:
SEQ ID NO: 8
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
or a sequence that is >95% identical to it,
and comprises the following sequence as a light chain:
SEQ ID NO: 9
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
or a sequence that is >95% identical to it.

4. Pharmaceutical aqueous formulation comprising an human or humanized anti-adrenomedullin (ADM) antibody or an anti-adrenomedullin antibody fragment according to claim 1, wherein said surfactant is Poloxamer.

5. Pharmaceutical aqueous formulation comprising an human or humanized anti-adrenomedullin (ADM) antibody or an anti-adrenomedullin antibody fragment according to claim 1, wherein Arginine is present in a range of 1 g/L to 100 g/L, preferably 15 g/L to 60 g/L, preferably 13.1 g/L to 52.2 g/L, preferably 11.2 g/L to 44.4 g/L and more preferably 26.1 g/L.

6. Pharmaceutical aqueous formulation comprising an human or humanized anti-adrenomedullin (ADM) antibody or an anti-adrenomedullin antibody fragment according to claim 1, wherein Trehalose is present in a range of 1 g/L to 100 g/L, preferably 32.6 g/L to 97.8 g/L, preferably 28.4 g/L to 85.1 g/L, preferably 24.1 g/L to 72.4 g/L and more preferably 56.7 g/L.

7. Pharmaceutical aqueous formulation comprising an human or humanized anti-adrenomedullin (ADM) antibody or an anti-adrenomedullin antibody fragment according to claim 1, wherein Poloxamer is present in a range of 0.01 g/L to 5 g/L, preferably 0.115 g/L to 1.15 g/L, preferably 0.1 g/L to 1.0 g/L, preferably 0.085 g/L to 0.85 g/L and more preferably 0.5 g/L.

8. Pharmaceutical aqueous formulation comprising an human or humanized anti-adrenomedullin (ADM) antibody or an anti-adrenomedullin antibody fragment according to claim 1, wherein Histidine is present in a range of 0.1 g/L to 6.4 g/L, preferably 0.22 g/L to 6.88 g/L, preferably 0.8 g/L to 3.2 g/L, preferably 0.68 g/L to 3.68 g/L and more preferably 1.6 g/L.

9. Pharmaceutical aqueous formulation comprising an human or humanized anti-adrenomedullin (ADM) antibody or an anti-adrenomedullin antibody fragment according to claim 1, wherein the pH of the formulation is in the range of 4.0 to 8.0 preferably 5.0 to 7.0, more preferably 6.0.

10. Pharmaceutical aqueous formulation comprising an human or humanized anti-adrenomedullin (ADM) antibody or an anti-adrenomedullin antibody fragment according to claim 1, wherein said formulation comprises an Anti-Adrenomedullin antibody directed to the N-terminal end of Adrenomedullin comprising the following sequence: SEQ ID No. 1, Arginine in a concentration of 26.1 g/L, Trehalose in a concentration of 56.7 g/L, Poloxamer in a concentration of 0.5 g/L, Histidine in a concentration of 1.6 g/L and wherein said formulation exhibits a pH of 6,0.

11. Pharmaceutical lyophilized formulation obtainable from a pharmaceutical aqueous formulation according to claim 1.

12. Method of making a ready-for-application solution comprising the steps:
a. Providing a pharmaceutical aqueous formulation according to claim 1
b. Adjusting the pharmaceutical aqueous formulation from step a) in a physiological acceptable solution by optionally diluting the volume of the pharmaceutical formulation from step a) with a physiological acceptable solution and by optionally aliquoting the pharmaceutical formulation from step a) wherein the diluted or adjusted aqueous pharmaceutical formulation is suitable for the administration in a patient.

13. Method of making a ready-for-application solution comprising the steps:
a. Providing a lyophilized formulation obtained from the aqueous formulation according to claim 1 by freeze drying optionally without adding any other bulk reagent
b. Reconstitution of the lyophilizate from step a) in water and/or a physiological acceptable solution for injection
c. Adjusting the reconstituted formulation from step b) in a physiological acceptable solution by optionally diluting the volume of the reconstituted formulation from step b) with a physiological acceptable solution and by optionally aliquoting the reconstituted formulation from step b) wherein the diluted or adjusted aqueous pharmaceutical formulation is suitable for the administration in a patient.

14. Ready-for-application aqueous pharmaceutical formulation obtainable by a method according to claim 12 comprising said human or humanized anti-adrenomedullin (ADM) antibody or an anti-adrenomedullin antibody fragment in a dose of 1 to 10 mg/kg body weight.

15. A method for the therapy or prevention of an acute disease or acute condition selected from the group comprising: SIRS, a severe infection, sepsis, shock selected from the group comprising shock due to hypovolemia, cardiogenic shock, obstructive shock and distributive shock, in particular cardiogenic shock, septic shock, shock due to Covid-19, shock due to burns and traumatic shock, acute vascular diseases as e.g. heart failure, congestion, in particular diuretic resistant congestion, inflammatory conditions, autoimmune diseases, metabolic diseases, brain diseases, cardiovascular diseases and drug-induced diseases symptoms of illness or an illness characterized by such symptoms, wherein the symptoms of illness are selected from the group of nausea, headache, muscle aches, back pain, shivering, and/or vomiting and migraine, comprising administering to a subject in need thereof an effective amount of the pharmaceutical formulation according to claim 1.

16. A method for the therapy or prevention of an acute disease or acute condition of a patient for prevention or reduction of organ dysfunction or prevention of organ failure in said patient, wherein said acute disease or acute condition is selected from the group comprising as e.g. severe infections, diabetes, cancer, acute and chronic vascular diseases as e.g. heart failure, myocardial infarction, stroke, atherosclerosis, shock and organ dysfunction, kidney dysfunction, liver dysfunction, burnings, surgery, traumata, poisoning and damages induced by chemotherapy and wherein said disease is not SIRS, sepsis or septic shock, comprising administering to said patient an effective amount of the pharmaceutical formulation according to claim 1.

17. A method for the therapy or prevention of an acute disease or acute condition of a patient for the regulation of fluid balance, wherein said patient is a patient in need of regulating the fluid balance and suffers from a disease that is selected from the group comprising systemic inflammatory Response-Syndrome (SIRS), sepsis, diabetes, cancer, heart failure, shock and kidney dysfunction, comprising administering to said patient an effective amount of the pharmaceutical formulation according to claim 1.

18. A method for the therapy or prevention of congestion in a patient wherein said patient has a disease or condition selected from the group comprising: congestive high blood pressure, swelling or water retention (edema), heart failure in particular acute heart failure, kidney or liver disease, comprising administering to said patient an effective amount of the pharmaceutical formulation according to claim 1.

19. A method for patient stratification for a therapeutic use by the pharmaceutical formulation according to claim 1, comprising:
determining the level of a fragment of pre-pro-Adrenomedullin selected from the group comprising Midregional Proadrenomedullin (MR-proADM), C-terminal Proadrenomedullin (CT-proADM), Bio-ADM, ADM-gly, or Proadrenomedullin N-terminal 20 peptide (PAMP) or fragments thereof prior to drug administration in a bodily fluid obtained from said subject; and
comparing said level of a fragment of pre-pro-Adrenomedullin selected from the group comprising MR-proADM, CT-proADM, PAMP, Bio-ADM or ADM-gly to a predefined threshold or to a previously determined level of said fragment, and
wherein for the correlation an elevated level of said fragment of pre-pro-Adrenomedullin selected from the group comprising MR-proADM, CT-proADM, PAMP, Bio-ADM or ADM-gly or fragments thereof above a certain threshold or above a previously determined level is used for patient stratification for a therapeutic use.

20. A method for the therapy of a patient with shock, in particular septic shock, wherein said patient:
has suffered from shock, in particular from a septic shock not longer than 8.4 hours at the starting point of treatment with said Anti-adrenomedullin (ADM) antibody or an anti-adrenomedullin antibody fragment and/or
has been admitted to ICU not longer than 8.4 hours at the starting point of treatment with said Anti-adrenomedullin (ADM) antibody or an anti-adrenomedullin antibody fragment, and/or
has not received organ support at all or not longer than 8.4 hours of organ support at the starting point of treatment with said Anti-adrenomedullin (ADM) antibody or an anti-adrenomedullin antibody fragment,
comprising administering to said patient an effective amount of the pharmaceutical formulation according to claim 1.

21. A method according to claim 19, wherein said patient is characterized by having a level of dipeptidyl peptidase 3 (DPP3) in a sample of bodily fluid of said patient below a threshold prior to drug administration.
